(19) United States
(12) Patent Application Publication
(10) Pub. No.: US 2024/0254085 A1
(43) Pub. Date: Aug. 1, 2024

(54) CHELATORS FOR RADIOMETALS AND METHODS OF MAKING AND USING SAME

(71) Applicant: The University of British Columbia, Vancouver (CA)

(72) Inventors: Luke WHARTON, Vancouver (CA); Chris ORVIG, Vancouver (CA)

(21) Appl. No.: 18/559,132

(22) PCT Filed: May 6, 2022
(86) PCT No.: PCT/CA2022/050712

§ 371 Date: Nov. 6, 2023

Related U.S. Application Data
(60) Provisional application No. 63/185,951, filed on May 7, 2021.

(51) Int. Cl. C07D 213/79; A61K 51/10

(52) U.S. Cl. CPC C07D 213/79; A61K 51/1093

(57) ABSTRACT

A chelator having the general structure (I) wherein each R1 is independently OH, NH or SH, and X is O, S, or NR3, wherein R3 is H or CH2C(═O)R1. Methods of making and using the chelator, metal chelates, and biological constructs for delivering targeted radiation therapy using the chelator are provided. (I).

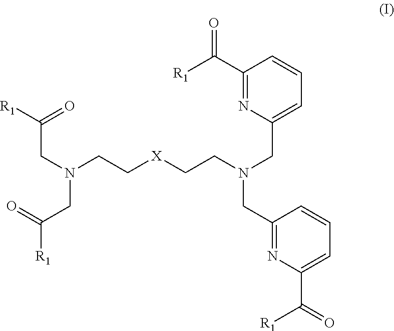

### CROSS-REFERENCE TO RELATED APPLICATIONS

This application claims priority to, and the benefit of, U.S. provisional patent application No. 63/185,951 filed 7 May 2021, the entirety of which is incorporated by reference herein for all purposes.

### TECHNICAL FIELD

Some embodiments relate to improved chelators. Some embodiments relate to improved biological targeting constructs incorporating chelators. Some embodiments relate to chelators coupled to a targeting moiety and capable of binding a radioactive isotope to provide targeted in vivo delivery of the radioactive isotope to a desired location within a mammalian subject.

### BACKGROUND

Radionuclides have potential utility in cancer diagnosis and therapy, particularly if they can be delivered selectively to a target location within the body of a subject. Targeted delivery of radionuclides can be achieved by using constructs that are engineered to both securely retain the radionuclide for in vivo delivery and deliver the radionuclide selectively to a desired location within the body, with a reasonably low level of delivery to non-target regions of the body.

Targeting constructs have been developed that utilize a targeting moiety that targets a desired region of the body (e.g. a tumor-associated antigen) covalently coupled to a chelator to secure radionuclides for such purposes. The targeting moiety can be coupled to the chelator via a linker. Such targeting constructs may be referred to as radioimmunoconjugates. The radioimmunoconjugate is used to chelate a desired radionuclide for in vivo delivery, for example to provide diagnostic imaging, targeted radionuclide therapy using the construct, or both (i.e. as a theranostic construct). In some cases, the chelator can also be used for in vivo delivery of a suitable companion radionuclide for diagnostics.

Historically, approaches towards the design of chelating ligands with high metal ion selectivity has been based on several fundamental principles, such as the selection of appropriate donor groups based upon Pearson's Hard and Soft Acids and Bases theory, the influence of cavity size, consideration of the macrocyclic/chelate effect, the optimal chelate ring size, etc. However, the influence of inverted donor group distribution on a common backbone has yet to be investigated. Given the inherent challenges in synthesis of ligands with symmetric donor substitution, and the requirements to often ‘break’ symmetry in the pursuit of bifunctional derivatives suitable for pharmaceutical application, an assessment of the exact impacts of asymmetric/inverse substitution on metal ion chelation is warranted. The development of new ligand systems with ‘inverted’ symmetry offers several advantages with respect to synthesis; protecting group strategies can be circumvented through rational synthetic design and the incorporation of bifunctional handles within the core framework is synthetically more accessible.

H₄noneunpa having the structure (A) has been previously published by the inventors,⁸ and was also published as Oxyaapa by Hu et al.¹³

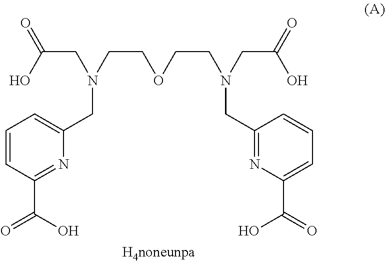 [SMILES: O=C(O)CN(CCOCCN(CC(=O)O)Cc1cccc(C(=O)O)n1)Cc1cccc(C(=O)O)n1]

In the context of nuclear medicine, significant improvements in the treatment of oncogenic disease have been achieved through the advent of several peptide-based radiopharmaceutical agents targeting somatostatin receptor subtype 2 (SSTR2) which is commonly overexpressed in neuroendocrine tumours (NETs).¹⁴ [¹¹¹In][In(DTPA-D-Phe-octreotide)] (OctreoScan™) and [⁶⁸Ga][Ga(DOTATATE)] are two established SSTR2 agonists used clinically for diagnosis of NETs.¹⁴ Most notably, [¹⁷⁷Lu][Lu(DOTATATE)](Lutathera®) reached clinical FDA-approval as a peptide receptor radionuclide therapy (PRRT) for the treatment of gastroenteropancreatic NETs in 2018.¹⁵ However, as a result of the inherent nuclear decay characteristics of β⁻-emitting radionuclides [low linear energy transfer (˜0.2 keV/μm), long-range (0.5-10 mm)] treatment scope in this context is limited to primary tumour sites and large metastases.^{4,16}

Targeted alpha therapy (TAT) has been highlighted as a powerful tool for treatment of both primary and metastatic tumours, with [²²⁵Ac]Ac³⁺ being one of the most prominent candidates for translation into clinical practice, owing to its compatible half-life (t_{1/2}=10 days), and the high therapeutic potency of its decay progeny (E_{α}=5-9 MeV, 50-100 μm range).¹⁷ For realisation of this objective, high denticity chelating ligands are required for stable chelation of the [²²⁵Ac]Ac³⁺ ion, which are further capable of sequestering imaging isotopes for diagnostics and assessment of patient suitability prior to radionuclide therapy. One potential imaging companion is the radio-lanthanide [¹⁵⁵Tb]Tb³⁺, which shares similar bonding characteristics to [²²⁵Ac]Ac³⁺ (e.g. CN=9; ionic radius=1.095 Å (Tb³⁺), 1.220 Å (Ac³⁺))^{18,19} and would be suitable for SPECT/CT diagnostics owing to its low-energy gamma emissions (E_{γ}=87 keV (32%), 105 keV (25%)) and comparable half-life (t_{1/2}=5.32 days).²⁰ Additionally, [¹⁵⁵Tb]Tb³⁺ is part of the so-called ‘Terbium theranostic quartet’ which includes the radioisotopes ¹⁴⁹Tb, ¹⁵²Tb, ¹⁵⁵Tb, and ¹⁶¹Tb, and encompasses both PET and SPECT imaging modalities, and all three therapeutic decay types (α, β⁻, Meitner-Auger electrons).²¹⁻²³ Thus, interchange between different Tb³⁺ radioisotopes would provide a true theranostic radiopharmaceutical with identical pharmacokinetic and biodistribution properties.

There is a general desire for improved chelators that are useful in the targeted in vivo delivery of radiometals using suitable targeting constructs.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

A novel chelator having the general structure (I) is provided wherein each R₁ is independently OH, NH or SH, and X is O, S, or NR₃, wherein R₃ is H or CH₂C(═O)R₁. In some embodiments, the chelator is a bifunctional molecule having the structure (II) wherein each R₁ is independently OH, NH or SH or a functional group, each R₂ is independently H or a functional group, and X is O, S, or NR₃, wherein one or more of the R₁ or R₂ groups is a functional group that allows coupling of the chelator to a biological targeting moiety, and wherein R₃ is H or CH₂C(═O)R₁. In some embodiments, the functional group is an ester, an amide, an imide, a thioamide, a thioester, or a guanidinium group. In some embodiments, a radioisotope targeting construct has the structure (II), wherein the chelator is coupled to a biological targeting moiety through one R₁ group or one R₂ group.

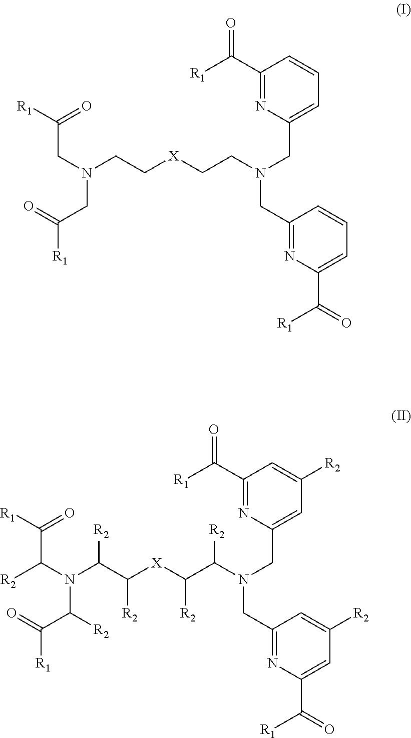

In some embodiments, the chelator has the structure (III) or is a bifunctional molecule having the structure (III) wherein each R₁ is independently OH, NH or SH or a functional group; each R₂ is independently H or a functional group; each R₄ is independently H or a functional group, or both R₄ together form a cyclohexyl moiety; each R₅ is independently H or a functional group, or both R₅ together form a cyclohexyl moiety; and X is O, S, or NR₃, wherein one or more of the R₁, R₂, R₄ or R₅ groups is a functional group that allows coupling of the chelator to a biological targeting moiety, and wherein R₃ is H or CH₂C(═O)R₁. In some embodiments, the functional group is an ester, an amide, an imide, a thioamide, a thioester, or a guanidinium group. In some embodiments, a radioisotope targeting construct has the structure (III), wherein the chelator is coupled to a biological targeting moiety through one R₁ group, one R₂ group, one R₄ group or one R₅ group.

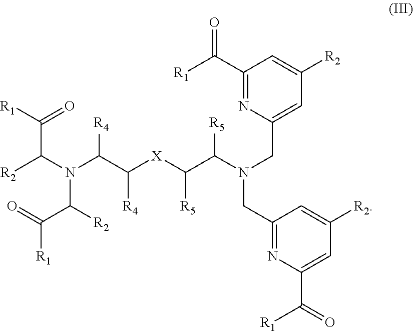

The chelator can be coupled to a biological targeting moiety to facilitate the targeted in vivo delivery of a radioisotope. The radioisotope can be ²²⁷Th, ²²⁵Ac, ¹⁵⁵Tb, ¹⁷⁷Lu, ¹¹¹In, ¹³²La, ²³⁵La, ⁹⁰Y, ⁶⁸Ga, ⁴⁴Sc, ²⁰³Pb, ²¹²Pb, or the like.

In some embodiments, the chelator has the following structure (6):

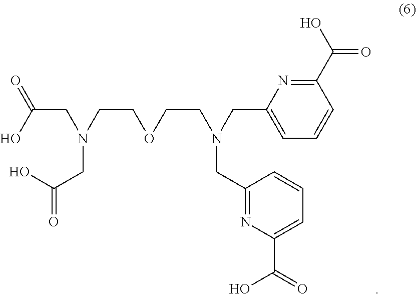 [SMILES: O=C(O)CN(CCOCCN(Cc1cccc(C(=O)O)n1)Cc1cccc(C(=O)O)n1)CC(=O)O]

Methods of administering an in vivo radioisotope targeting construct comprising the chelator are provided. The in vivo radioisotope targeting construct can be administered to a mammalian subject. The targeting moiety of the in vivo radioisotope targeting construct can be used to enhance accumulation of the radioisotope at a selected location within the body (e.g. the location of cancerous cells), relative to other locations in the body. An imaging procedure can be carried out to evaluate the localization of the in vivo radioisotope targeting construct within the body. The in vivo radioisotope targeting construct can be used to cause cell death at the selected location within the body by exposing the cells to radiation from the radioisotope. The cells can be cancer cells. The mammalian subject can be a human.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 shows the structure of an exemplary in vivo targeting chelate construct.

FIG. 2 shows the chemical structure of H₄noneunpaX (6) and the ¹H assignment (left side) and ¹³C assignment (right side).

FIG. 3 shows the ¹H NMR spectrum of H₄noneunpaX·4HCl (6) (400 MHz, D₂O, 298 K).

FIG. 4 shows ¹³C {¹H} NMR spectrum of H₄noneunpaX·4HCl (6) (75 MHz, D₂O, 298 K).

FIG. 5 shows ¹H-¹H COSY NMR spectrum of H₄noneunpaX (6) (300 MHz, D₂O, 298 K).

FIG. 6 shows ¹H-¹³C HSQC NMR spectrum of H₄noneunpaX 4HCl (6) (300 MHz, 75 MHz, D₂O, 298 K).

FIG. 7 shows ¹H-¹³C HMBC NMR spectrum of H₄noneunpaX 4HCl (6) (300 MHz, 75 MHz, D₂O, 298 K).

FIG. 8 shows stacked ¹H NMR spectra of H₄noneunpaX and corresponding complexes of La³⁺, Lu³⁺ and In³⁺ (400 MHz, D₂O, 298 K, pD 7.0). [In(noneunpaX)]⁻ was characterised at pD=4.5.

FIG. 9 shows ¹H NMR spectrum of [La(noneunpaX)]⁻ (400 MHz, D₂O, 298 K, pH 7), and includes the chemical structure of [La(noneunpaX)]⁻ with ¹H assignments indicated.

FIG. 10 shows ¹H-¹H COSY NMR spectrum of [La(noneunpaX)]⁻ (400 MHz, D₂O, 298 K, pH 7.0).

FIG. 11 shows ¹H-¹H COSY NMR spectrum of [La(noneunpaX)]⁻ (400 MHz, D₂O, 298 K, pH 7.0) (alkyl expansion).

FIG. 12 shows ¹H-¹H COSY NMR spectrum of [La(noneunpaX)]⁻ (400 MHz, D₂O, 298 K, pH 7.0) (aromatic expansion).

FIG. 13 shows ¹H NMR spectrum of [Lu(noneunpaX)]⁻ (400 MHz, D₂O, 298 K, pH 7.0) and includes the chemical structure of [Lu(noneunpaX)]⁻ with ¹H assignments indicated.

FIG. 14 shows ¹H-¹H COSY NMR spectrum of [Lu(noneunpaX)]⁻ (400 MHz, D₂O, 298 K, pH 7.0).

FIG. 15 shows ¹H-¹H COSY NMR spectrum of [Lu(noneunpaX)]⁻ (400 MHz, D₂O, 298 K, pH 7.0) (alkyl expansion).

FIG. 16 shows ¹H-¹H COSY NMR spectrum of [Lu(noneunpaX)]⁻ (400 MHz, D₂O, 298 K, pH 7.0) (aromatic expansion).

FIG. 17 shows ¹H NMR spectrum of [In(noneunpaX)]⁻ (400 MHz, D₂O, 298 K, pD 4.5) and includes the chemical structure of [In(noneunpaX)]⁻ with ¹H assignments indicated.

FIG. 18 shows ¹H-¹H COSY NMR spectrum of [In(noneunpaX)]⁻ (400 MHz, D₂O, 298 K, pD 4.5).

FIG. 19 shows calculated absorptivity curves of the eight species of H₄noneunpaX; determined by combined potentiometric-spectrophotometric titrations ([L]=9.57×10⁻⁴ M, I=0.2 cm), and acidic in-batch spectrophotometric titrations ([L]=1.0×10⁻⁴ M, I=1.0 cm), at T=298 K, I=0.16 M NaCl.

FIG. 20 shows a speciation diagram of H₄noneunpaX as a function of pH, dotted line indicates pH 7.4.

FIG. 21 shows a speciation diagram of H₄noneunpaX with La³⁺ ([La³⁺]=[H₄noneunpaX]=1×10⁻⁴ M), dashed lines indicated physiological conditions (pH 7.4), at 298 K, I=0.16 M NaCl.

FIG. 22 shows a speciation diagram of H₄noneunpaX with Sm³⁺ ([Sm³⁺]=[H₄noneunpaX]=1×10⁻⁴ M), dashed lines indicated physiological conditions (pH 7.4), at 298 K, I=0.16 M NaCl.

FIG. 23 shows a speciation diagram of H₄noneunpaX with Gd³⁺ ([Gd³⁺]=[H₄noneunpaX]=1×10⁻⁴ M), dashed lines indicated physiological conditions (pH 7.4), at 298 K, I=0.16 M NaCl.

FIG. 24 shows a speciation diagram of H₄noneunpaX with Dy³⁺ ([Dy³⁺]=[H₄noneunpaX]=1×10⁻⁴ M), dashed lines indicated physiological conditions (pH 7.4), at 298 K, I=0.16 M NaCl.

FIG. 25 shows a speciation diagram of H₄noneunpaX with Lu³⁺ ([Lu³⁺]=[H₄noneunpaX]=1×10⁻⁴ M), dashed lines indicated physiological conditions (pH 7.4), at 298 K, I=0.16 M NaCl.

FIG. 26 shows a speciation diagram of H₄noneunpaX with In³⁺ ([In³⁺]=[H₄noneunpaX]=1×10⁻⁴ M), dashed lines indicated physiological conditions (pH 7.4), at 298 K, I=0.16 M NaCl.

FIG. 27 shows a speciation diagram of H₄noneunpaX with Sc³⁺ ([Sc³⁺]=[H₄noneunpaX]=1×10⁻⁴ M), dashed lines indicated physiological conditions (pH 7.4), at 298 K, I=0.16 M NaCl.

FIG. 28 shows concentration-dependent radiolabelling studies of H₄noneunpa, H₄noneunpaX, and DOTA with: [⁴⁴Sc]Sc³⁺ (1.2 MBq) in NaOAc (0.1 M, pH 4.5) (panel (A)), [¹¹¹In]In³⁺ (1.0 MBq) in NH₄OAc (0.5 M, pH 5.8) (panel (B)), [¹⁵⁵Tb]Tb³⁺ (40 kBq) in NH₄OAc (0.5 M, pH 6.0) (panel (C)), [¹⁷⁷Lu]Lu³⁺ (150 kBq) in NH₄OAc (0.5 M, pH 6.0) (panel (D)), [²¹³Bi]Bi³⁺ (680 kBq) in MES (1.0 M, pH 5.5), (F) [²²⁵Ac]Ac³⁺ (40 kBq) in NH₄OAc (1.0 M, pH 7.3) (panel (E)).

FIG. 29 shows human serum stability studies of H₄noneunpa and H₄noneunpaX with: [¹¹¹In]In³⁺ (54 GBq/μmol) (panel (A)), [¹⁵⁵Tb]Tb³⁺ (1.0 GBq/μmol) (panel (B)), [¹⁷⁷Lu]Lu³⁺ (2.0 GBq/μmol) (panel (C)), [²²⁵Ac]Ac³⁺ (134 MBq/μmol) (panel (D)).

FIG. 30 shows DFT-optimised structures of [La(noneunpaX)]⁻ in panel (A) (left-hand orientation), and panel (B) front orientation, and [Lu(noneunpaX)]⁻ in panel (c) (left-hand orientation), and panel (D) (front orientation). Selected hydrogens have been omitted for clarity.

FIG. 31 shows concentration-dependent radiolabelling of bifunctional H₄noneunpaX with: (A) [⁴⁴Sc]Sc³⁺ (400 kBq) in NaOAc (0.1 M, pH 4.5), (B) [¹¹¹In]In³⁺ (1.06 MBq) in NH₄OAc (0.5 M, pH 5.5), (C) [¹⁷⁷Lu]Lu³⁺ (350-500 kBq) in NH₄OAc (0.5 M, pH 6.0), (D) [^{133/135}La]La³⁺ (400 kBq) in NH₄OAc (0.2 M, pH 7.0), (E) [¹⁵⁵Tb]Tb³⁺ (140 kBq) in NH₄OAc (0.5 M, pH 7.0) and (F) [²²⁵Ac]Ac³⁺ (40 kBq) in NH₄OAc (0.5 M, pH 7.2).

FIG. 32 shows a molar activity study of H₄noneunpaX-Bn-NH₂ with [¹⁷⁷Lu]LuCl₃ (20 MBq) in NH₄OAc (0.5 M, pH 6.0) at RT monitored over 10 minutes. Highest molar activity (250 GBq/μmol), ligand-to-metal ratio ([L]:[M]; [174:1]) (left panel). Human serum stability challenge of [¹⁷⁷Lu][Lu(noneunpaX-Bn-NH₂]⁻ (5.2 GBq/μmol) conducted at 37° C. and monitored over 7 days via radio-iTLC (n=3) (right panel).

FIG. 33 shows radiolabelling studies of bifunctional H₄noneunpaX and corresponding peptide-conjugates: (A) Concentration-dependent radiolabelling with [²²⁵Ac]Ac³⁺ (40 kBq) in NH₄OAc buffer (0.5 M, pH 7) (RT, 10 min.). (B) Human serum stability challenge of [²²⁵Ac]Ac³⁺-labelled compounds. (C) Concentration-dependent radiolabelling with [¹⁵⁵Tb]Tb³⁺ (120 kBq) in NH₄OAc buffer (0.5 M, pH 6) (RT, 10 min.). (D) Human serum stability challenge of [¹⁵⁵Tb]Tb³⁺-labelled bioconjugates.

FIG. 34 shows radio-HPLC traces of [¹⁵⁵Tb]Tb³⁺ control and [¹⁵⁵Tb][Tb³⁺ labelled H₄noneunpaX-Bn-NH₂ and H₄noneunpaX-Bn-NCS. Method: A: H₂O (0.1% TFA), B: MeCN (0.1% TFA); 100% A to 10% B over 15 min., 1 mL/min.

FIG. 35 shows radio-HPLC traces for [¹⁵⁵Tb][Tb(noneunpaX-Ahx-Tyr³-TATE)], Method: A: H₂O (0.1% TFA), B: MeCN (0.1% TFA); 100% A to 60% B over 15 min., 1 mL/min.

FIG. 36 shows radio-HPLC traces for [¹⁵⁵Tb][Tb(noneunpaX-PEG₂-Tyr³-TATE)], Method: A: H₂O (0.1% TFA), B: MeCN (0.1% TFA); 100% A to 60% B over 15 min., 1 mL/min.

FIG. 37 shows coronal views of maximum intensity projections (MIPs) from quantitative dynamic SPECT/CT scans at 0-1 h post administration of [¹⁵⁵Tb][Tb(noneunpaX-Ahx-Tyr³-TATE)] (top) and [¹⁵⁵Tb][Tb(noneunpaX-PEG₂-Tyr³-TATE)](bottom) in NRG mice bearing AR42J exocrine tumour xenografts (left shoulder).

FIG. 38 shows sagittal views of maximum intensity projections (MIPs) from SPECT/CT scans at 1, 2 and 4 h post administration of [¹⁵⁵Tb][Tb(noneunpaX-Ahx-Tyr³-TATE)] (top) and [¹⁵⁵Tb][Tb(noneunpaX-PEG₂-Tyr³-TATE)] (bottom) in NRG mice bearing AR42J pancreatic exocrine tumour xenografts (left shoulder).

FIG. 39 shows representative time-activity plots of (A) [¹⁵⁵Tb][Tb(noneunpaX-Ahx-Tyr³-TATE)] and (B) [¹⁵⁵Tb][Tb(noneunpaX-PEG-Tyr³-TATE)] in NRG mice bearing AR42J exocrine/pancreatic tumour xenografts. Standardised uptake values (SUVs) were extracted for ROIs in relevant organs from calibrated SPECT/CT images.

FIG. 40 shows decay-corrected biodistribution studies of [¹⁵⁵Tb][Tb(noneunpaX-Ahx-Tyr³-TATE)] at 2 h and 4 h post-injection and [¹⁵⁵Tb][Tb(noneunpaX-PEG₂-Tyr³-TATE)] at 4 h post-injection in NRG mice bearing AR42J exocrine/pancreatic tumour xenografts.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

The inventors have now developed a novel chelating ligand, H₄noneunpaX, having an inverted arrangement of functional groups, and have compared the characteristics of this novel ligand with H₄noneunpa. The ligand displays preferential complexation with trivalent Ln³⁺ ions, forming single isomeric species in solution, which exhibits high thermodynamic stability and kinetic inertness. Radiolabelling studies of H₄noneunpaX with [¹¹¹In]In³⁺, [¹⁵⁵Tb]Tb³⁺, [¹⁷⁷Lu]Lu³⁺ and [²²⁵Ac]Ac³⁺ showed excellent compatibility, achieving high molar activities under mild conditions (RT, 10 mins). As a proof of principle, a bifunctional derivative of H₄noneunpaX was prepared through a facile synthetic approach and was conjugated to two Tyr³-Octreotate peptide analogues for targeting of neuroendocrine tumours (NETs). Further radiolabelling studies of these bioconjugates with [¹¹¹In]In³⁺, [¹⁷⁷Lu]Lu³⁺, [^{133/135}La]La³⁺, [¹⁵⁵Tb]Tb³⁺ and [²²⁵Ac]Ac³⁺, showed promising results for the development of an [²²⁵Ac]Ac³⁺/[¹⁵⁵Tb]Tb³⁺-theranostic pair. SPECT/CT and biodistribution studies of the [¹⁵⁵Tb]Tb³⁺-radiolabelled tracers in tumour bearing mice, showed excellent performance, with good tumour uptake, low residual background organ activity, and no evidence of degradation in vivo.

The objectives of this work were therefore to investigate the influence of donor group substitution about a common backbone and study the impact of asymmetric/inverted displacement on metal ion affinity. H₄noneunpaX having the structure (6) was prepared and comparisons to the symmetric counterpart H₄noneunpa/H₄oxyaapa were assessed. To further assess the utility of this approach, a bifunctional analogue of H₄noneunapX was prepared, to firstly demonstrate the synthetic accessibility of bifunctional asymmetric/inverted chelators, and to investigate their performed in vivo.

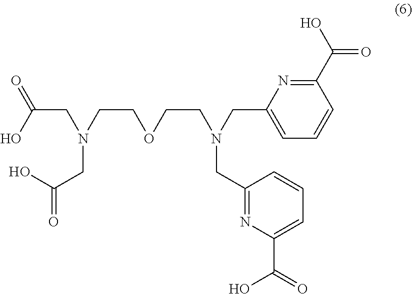 [SMILES: O=C(O)CN(CCOCCN(Cc1cccc(C(=O)O)n1)Cc1cccc(C(=O)O)n1)CC(=O)O]

As used herein, the term prophylaxis includes preventing, minimizing the severity of, or preventing a worsening of a condition. As used herein, the terms treat or treatment include reversing or lessening the severity of a condition.

As used herein, the term antibody includes all forms of antibodies including polyclonal antibodies, monoclonal antibodies, chimeric antibodies, humanized antibodies, single chain antibodies, multimeric antibodies, and the like. The term antigen binding fragment of an antibody refers to any portion of an antibody that is capable of binding to an antigen and includes by way of example only and without limitation Fab fragments, F(ab′)₂ fragments, Fv fragments, scFv fragments, minibodies, diabodies, and the like. Reference to a specific antibody includes reference to any antibodies that are determined to be biosimilar to that specific antibody by any regulatory authority.

As used herein, the term peptidomimetic means a small protein-like molecule designed to mimic a peptide, and includes without limitation modified peptides, peptidic foldamers, structural mimetics and mechanistic mimetics.

A chelator composition for radiometals is disclosed. A method of using and making the composition is also disclosed. The composition can be used as a therapeutic and/or diagnostic agent.

The inventors have now determined that chelators having the general structure (6) can coordinate radioisotopes under mild conditions and produce a complex that is stable under in vivo conditions, making such chelators particularly suitable for example for application in radiotherapeutic, diagnostic and/or theranostic constructs. The chelator can be coupled directly or via a linker to a biological targeting moiety to create a construct suitable for use in such applications.

In some embodiments, H₄noneunpaX can be directly coupled to a biological targeting moiety, optionally with a linker interposing the H₄noneunpaX and the biological targeting moiety, by coupling the biological targeting moiety or linker directly to one of the carboxyl groups of structure (6). Furthermore, in some embodiments, one or more of the oxygen atoms of the carboxyl group is substituted by a different heterotatom, e.g. N or S. Thus, in various embodiments, the functional group provided on the bifunctional H₄noneunpaX chelator to couple the chelator to the biological targeting moiety can be a carboxyl, an ester, an amide, an imide, a thioamide, a thioester, a guanidinium, or the like.

In some embodiments, the chelator has the following structure (1) wherein each R₁ is independently OH, NH or SH, and wherein X is O, S, or NR₃, wherein R₃ is H or CH₂C(═O)R₁.

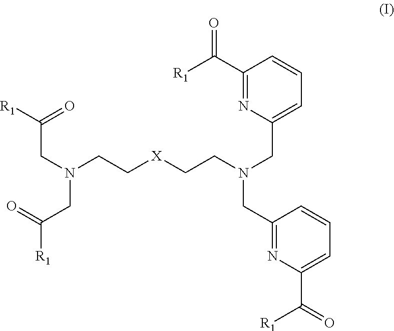

In some embodiments, the chelator is a bifunctional molecule having the structure (II) wherein each R₁ is independently OH, NH or SH or a functional group, each R₂ is independently H or a functional group, and X is O, S, or NR₃, wherein one or more of the R₁ or R₂ groups is a functional group that allows coupling of the chelator to a biological targeting moiety, and wherein R₃ is H or CH₂C(═O)R₁.

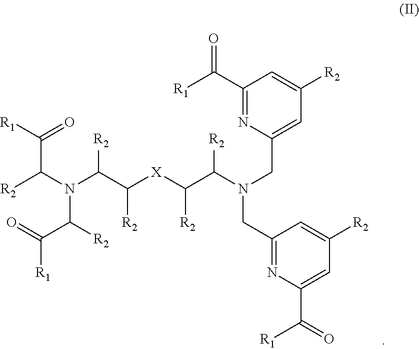

In some such embodiments, the functional group is a carboxyl, an ester, an amide, an imide, a thioamide, a thioester, a guanidinium, or the like.

In some embodiments, a radioisotope targeting construct has the structure (II), wherein one of the R₁ groups or one of the R₂ groups is a biological targeting moiety.

In some embodiments, the chelator has the structure (III) or is a bifunctional molecule having the structure (III) wherein each R₁ is independently OH, NH or SH or a functional group; each R₂ is independently H or a functional group; each R₄ is independently H or a functional group, or both R₄ together form a cyclohexyl moiety; each R₅ is independently H or a functional group, or both R₅ together form a cyclohexyl moiety; and X is O, S, or NR₃, wherein one or more of the R₁, R₂, R₄ or R₅ groups is a functional group that allows coupling of the chelator to a biological targeting moiety, and wherein R₃ is H or CH₂C(═O)R₁. In some embodiments, when both R₄ together form a cyclohexyl moiety, each R₅ is independently H or a functional group. In some embodiments, when both R₅ together form a cyclohexyl moiety, each R₄ is independently H or a functional group. In some embodiments, the functional group is an ester, an amide, an imide, a thioamide, a thioester, or a guanidinium group or the like. In some embodiments, a radioisotope targeting construct has the structure (III), wherein the chelator is coupled to a biological targeting moiety through one R₁ group, one R₂ group, one R₄ group or one R₅ group. In some embodiments, a radioisotope targeting construct has the structure (III), wherein one of the R₁ groups, one of the R₂ groups, one of the R₄ groups or one of the R₅ groups is a biological targeting moiety.

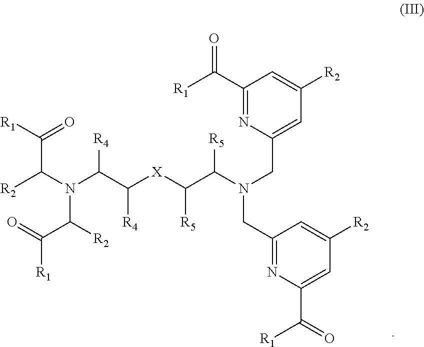

For example, in some embodiments, the chelator has one of the structures shown below:

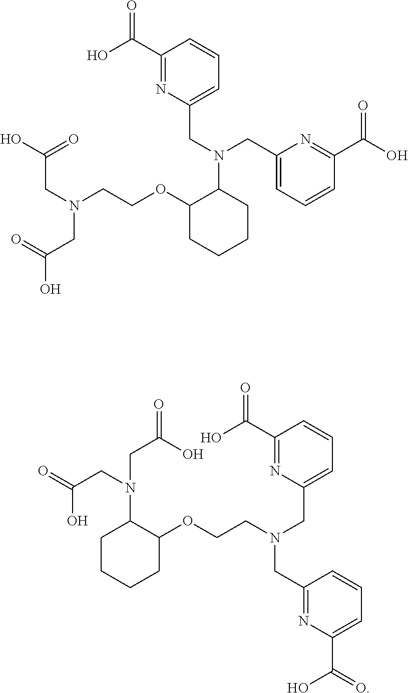 [SMILES: O=C(O)CN(CCOC1CCCCC1N(Cc1cccc(C(=O)O)n1)Cc1cccc(C(=O)O)n1)CC(=O)O ; O=C(O)CN(CC(=O)O)C1CCCCC1OCCN(Cc1cccc(C(=O)O)n1)Cc1cccc(C(=O)O)n1]

It has been found by other groups, for example Milenic et al.⁵⁸, that DTPA derivatives comprising a cyclohexyl moiety in the backbone of the chelator show comparable specific activities when incorporated into a chelate-antibody conjugate. Accordingly, it can be soundly predicted that compounds incorporating a cyclohexyl moiety in the backbone of the chelator will likely exhibit similar binding properties to NoneunpaX having the structure (6).

In some embodiments, the chelator is NoneunpaX having the structure (6).

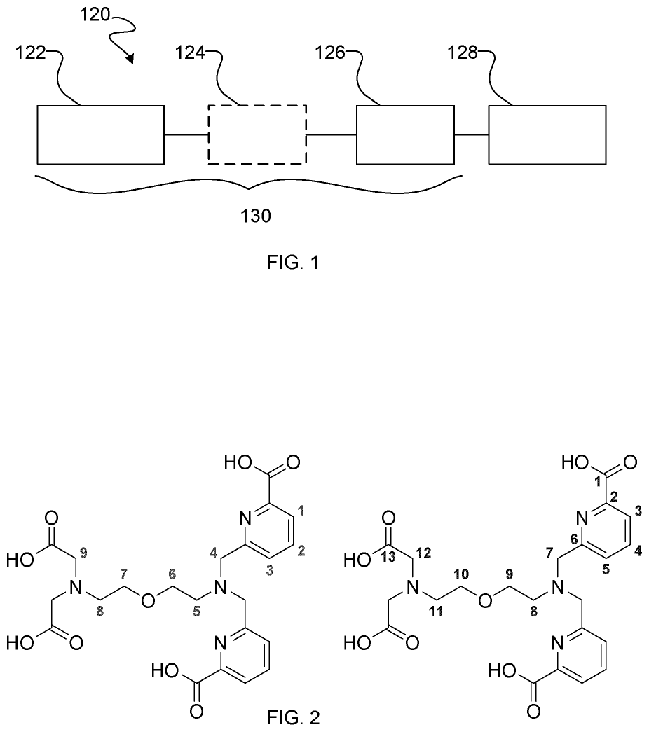

In some embodiments as shown in FIG. 1, an in vivo targeting chelate construct 120 has a targeting moiety 122 coupled to a chelator 126. In some embodiments, including the illustrated embodiment, a linker 124 interposes targeting moiety 122 and chelator 126. Together, targeting moiety 122, linker 124 (if present) and chelator 126 comprise in vivo targeting construct 130. Further, chelator 126 is used to chelate a radionuclide 128 that is suitable for in vivo imaging and/or radiotherapy. Radionuclide 128 together with in vivo targeting construct 130 provides an in vivo targeting chelate construct 120 suitable for targeted in vivo delivery of the radionuclide 128 payload as assisted by targeting moiety 122.

Any moiety suitable for directing the targeted delivery of in vivo targeting chelate construct 120 in vivo can be used as targeting moiety 122. In some embodiments, the targeting moiety 122 of the targeting construct 120 is a hapten, antigen, aptamer, affibody molecule, enzyme, protein, peptide, antibody, antigen-binding fragment of an antibody, peptidomimetic, receptor ligand, steroid, hormone, growth factor, cytokine, molecule that recognizes cell surface receptors (including molecules involved in growth, metabolism or function of cells), lipid, lipophilic group, carbohydrate, or any other molecule or targeting component capable of selectively directing a construct to a specific location within the body. The targeting moiety can be produced in any suitable manner, e.g. as a biologic, semisynthetically, or synthetically.

Examples of targeting moieties that have been developed to deliver radioisotope targeting constructs to desired locations within the body of a mammalian subject in vivo include antibodies targeting specific markers associated with specific types of cancers, peptidomimetics targeting proteins that are highly expressed in cancer cells, and the like. Exemplary non-limiting examples of suitable targeting moieties are listed in Table 1 (Lau, J.; Rousseau, E.; Kwon, D.; Lin, K.; Benard, F.; Chen, X., Insight into the Development of PET Radiopharmaceuticals for Oncology. Cancers 2020, 12, 1312, the entirety of which is incorporated by reference herein). Some targeting moieties selectively interact with biological targets, including antigens, proteins, carbohydrates or other molecules present on the surface of cells that are overexpressed in cancer cells relative to normal cells, e.g. tumor-associated antigens. Exemplary non-limiting examples of suitable targets are listed in Table 1. Suitable targets and/or targeting moieties for radiopharmaceuticals, whether now known or discovered or developed in the future, would be known to a person skilled in the art. In some embodiments, targeting moiety 122 is an antibody or an antigen-binding fragment of an antibody. In some embodiments, targeting moiety 122 is a peptidomimetic. In some embodiments, the targeting moiety 122 is one of the targeting moieties listed in Table 1, with any chelator present in the referenced molecule replaced by H₄noneunpaX as the chelator. In some embodiments, the targeting moiety 122 interacts selectively with one of the targets listed in Table 1.

TABLE 1
Exemplary targeting moieties and biological targets for targeted radiation therapy.

| Targeting Moiety | Biological Target | Indications |
|---|---|---|
| A33 antibody | A33 transmembrane glycoprotein | colorectal cancer |
| dihydrotestosterone (DHT) | androgen receptor (AR) | prostate cancer |
| HuMab-5B1 | CA19.9 | pancreatic cancer, bladder |
| | | cancer |
| Girentuximab | carbonic anhydrase 9 (CA-IX) | clear cell renal cell |
| | | carcinoma |
| AMG211 bispecific T-cell engager | carcinoembryonic antigen | gastrointestinal |
| | | adenocarcinoma |
| IAB22M2C minibody | CD8 | melanoma, lung cancer, |
| | | hepatocarcinoma |
| Rituximab | CD20 | B-cell lymphoma |
| obinutuzumab | CD20 | B-cell lymphoma |
| U36 antibody | CD44v6 | head and neck cancer |
| Plerixafor | C-X-C chemokine receptor type 4 | hematological and solid |
| | (CXCR4) | malignancies |
| Pentixafor | CXCR4 | hematological and solid |
| | | malignancies |
| NFB | CXCR4 | hematological and solid |
| | | malignancies |
| Ipilimumab | Cytotoxic T-lymphocyte- | Melanoma |
| | associated protein 4 (CTLA-4) | |
| erlotinib | epidermal growth factor receptor | nonsmall cell lung |
| | (EGFR) | carcinoma; colorectal cancer |
| PD153035 | EGFR | nonsmall cell lung |
| | | carcinoma; colorectal cancer |
| Afatinib | EGFR | nonsmall cell lung |
| | | carcinoma; colorectal cancer |
| Cetuximab | EGFR | nonsmall cell lung |
| | | carcinoma; colorectal cancer |
| panitumumab | EGFR | nonsmall cell lung |
| | | carcinoma; colorectal cancer |
| ABY-025 affibody | Epidermal growth factor receptor 2 | breast cancer |
| | (ERBB2) | |
| HER2-nanobody | ERBB2 | breast cancer |
| Trastuzumab | ERBB2 | breast cancer |
| Pertuzumab | ERBB2 | breast cancer |
| GSK2849330 | epidermal growth factor receptor 3 | solid malignancies |
| | (ERBB3) | |
| lumretuzumab | ERBB3 | solid malignancies |
| 4FMFES | estrogen receptor (ER) | breast cancer and |
| | | gynecologic cancers |
| FAPI-04 | fibroblast activation protein α | solid malignancies |
| FAPI-21 | fibroblast activation protein α | solid malignancies |
| FAPI-46 | fibroblast activation protein α | solid malignancies |
| Galactose | galactose metabolism | hepatocarcinoma |
| CB-TE2A-AR06 peptide (with | gastrin-releasing peptide receptor | prostate cancer, breast |
| H₄noneunpaX substituted for | (GRPR) | cancer, glioma |
| DOTA) | | |
| BAY 864367 peptide (with | GRPR | prostate cancer, breast |
| H₄noneunpaX -bound ligand label | | cancer, glioma |
| instead of 18F labeling) | | |
| RM2 peptide (with H₄noneunpaX | GRPR | prostate cancer, breast |
| substituted for DOTA) | | cancer, glioma |
| SB3 peptide (with H₄noneunpaX | GRPR | prostate cancer, breast |
| substituted for DOTA) | | cancer, glioma |
| RM26 peptide | GRPR | prostate cancer, breast |
| | | cancer, glioma |
| BBN-RGD peptide | GRPR | prostate cancer, breast |
| | | cancer, glioma |
| Aca-BBN peptide | GRPR | prostate cancer, breast |
| | | cancer, glioma |
| NeoBOMB1 peptide (with | GRPR | prostate cancer, breast |
| H₄noneunpaX substituted for | | cancer, glioma |
| DOTA) | | |
| exendin-4 peptide | glucagon-like peptide 1 receptor | insulinoma |
| | (GLP-1R) | |
| Glucose | glucose metabolism | neoplasm |
| Codrituzumab | glypican 3 | hepatocarcinoma |
| EF5 | Hypoxia | solid malignancies |
| MISO | Hypoxia | solid malignancies |
| AZA | Hypoxia | solid malignancies |
| HX4 | Hypoxia | solid malignancies |
| ASTM | Hypoxia | solid malignancies |
| LLP2A peptidomimetic | integrin α4β1 | multiple myeloma |
| galacto-RGD peptide | integrin αvβ3 | solid malignancies |
| FPP(RGD)2 peptide | integrin αvβ3 | solid malignancies |
| RGD-K5 peptide | integrin αvβ3 | solid malignancies |
| Fluciclatide | integrin αvβ3 | solid malignancies |
| alfatide-I | integrin αvβ3 | solid malignancies |
| alfatide-II | integrin αvβ3 | solid malignancies |
| PRGD2 peptide | integrin αvβ3 | solid malignancies |
| αvβ6-BP peptide | integrin αvβ6 | head and neck cancer, lung |
| | | cancer, colorectal cancer, |
| | | breast cancer, pancreatic |
| | | cancer |
| CycMSHhex targeting peptides | melanocortin-1 receptor (MC1R) | melanoma |
| MMOT0530A antibody | Mesothelin | pancreatic ductal |
| | | adenocarcinoma and |
| | | ovarian cancer |
| SP peptide | Neurokinin1 receptor (NK1R) | glioma |
| neurotensin | Neurotensin 1 receptor (NTS1R) | prostate cancer |
| PARPi | poly(ADP-ribose) polymerase 1 | head and neck cancer |
| | (PARP1) | |
| PSMA peptidomimetics, e.g. | Prostate-specific membrane | prostate cancer |
| PSMA-1007, PSMA-7 (with | antigen (PSMA) | |
| H₄noneunpaX -bound ligand | | |
| replacing 18F labelling); PSMA-11 | | |
| (with H₄noneunpaX replacing the | | |
| HBED chelator); PSMA-617, | | |
| PSMA-I&T (with H₄noneunpaX | | |
| replacing the DOTA chelator) | | |
| DCFPyL | PSMA | prostate cancer |
| DCFBC | PSMA | prostate cancer |
| HuJ591 antibody | PSMA | prostate cancer |
| durvalumab | programmed cell death protein | nonsmall cell lung |
| | (PD-1) | carcinoma |
| nivolumab | PD-1 | nonsmall cell lung |
| | | carcinoma |
| pembrolizumab | PD-1 | nonsmall cell lung |
| | | carcinoma |
| BMS-986192 adnectin | programmed death-ligand 1 (PD- | nonsmall cell lung |
| | L1) | carcinoma, bladder cancer, |
| | | breast cancer |
| atezolizumab | PD-L1 | nonsmall cell lung |
| | | carcinoma, bladder cancer, |
| | | breast cancer |
| MSTP2109A antibody | six-transmembrane epithelial | prostate cancer |
| | antigen of prostate-1 (STEAP1) | |
| TATE peptide (octreotate) | somatostatin receptor 2 (SSTR2) | neuroendocrine tumors |
| TOC peptide | SSTR2 | neuroendocrine tumors |
| NOC peptide | SSTR2 | neuroendocrine tumors |
| JR11 | SSTR2 | neuroendocrine tumors |
| thymidine | thymidine kinase (DNA replication) | solid malignancies |
| fresolimumab | transforming growth factor-beta | glioma |
| | (TGF-β) | |
| bevacizumab | vascular endothelial growth factor | solid malignancies |
| | receptor (VEGFR) | |

Any suitable linker can be used as linker 124 to couple chelator 126 to targeting moiety 122. For example and by way of illustration only, suitable linkers can include:
a hydrocarbon linker containing between 1 and 10 carbon atoms (C₁-C₁₀), including 2, 3, 4, 5, 6, 7, 8 or 9 carbon atoms that is optionally saturated or unsaturated, optionally substituted with one or more heteroatoms or having one or more substituents; the hydrocarbon linker can be linear, cyclic and/or branched, e.g. 8-aminooctanoic acid, 6-aminohexanoic acid;
an aromatic linker containing an aromatic moiety such as a benzyl group, e.g. aminophenylacetic acid;
an amino acid linker having between 1 and 10 amino acid residues, including 2, 3, 4, 5, 6, 7, 8, or 9 amino acid residues, any one or more of which may be naturally occurring amino acid residues, D-amino acid residues or other non-naturally occurring residues, examples of which include GlyGly, GluGluGlu, GlySerGlySer;
a cyclized linker, or cyclized ring structure, optionally a cyclized amino acid linker, e.g. aminocyclohexanecarboxylic acid;
a PEG-linker of any suitable length;
cationic linkers, whether formed from amino acid residues or other residues, e.g. Pip, 4-(2-aminoethyl)-1-carboxymethyl-piperazine (Acp);
anionic linkers, whether formed from amino acid residues or other residues, e.g. AspAsp, GluGlu;
a carbohydrate containing linker;
click chemistry linkers (triazoles);
any other suitable linker;
or combinations or modifications of the foregoing.

Examples of linkers that have been developed in the art for other radiopharmaceutical targeting constructs are known to those skilled in the art. Hydrophilic or charged linkers such as PEG-linkers or cationic/anionic linkers may be used to increase the overall water solubility of the targeting construct. Amino acid side chain substitutions and/or inclusion of carbohydrate moieties may be made to improve or alter the solubility and/or pharmacokinetics of the targeting construct A person skilled in the art could develop and optimize a suitable linker for a particular application if desired. Examples of linkers that have been developed in the art for other radiopharmaceutical targeting constructs are described, by way of example only and without limitation, by Benešová et al., Barnaski et al. and Kuo et al. (See Benešová, M.; Schäfer, M.; Bauder-Wüst, U.; Afshar-Oromieh, A.; Kratochwil, C.; Mier, W.; Haberkorn, U.; Kopka, K.; Eder, M., Preclinical Evaluation of a Tailor-Made DOTA-Conjugated PSMA Inhibitor with Optimized Linker Moiety for Imaging and Endoradiotherapy of Prostate Cancer. J. NucL Med. 2015, 56, 914-920; Baranski, A.-C.; Schäfer, M.; Bauder-Wüst, U.; Wacker, A.; Schmidt, J.; Liolios, C.; Mier, W.; Haberkorn, U.; Eisenhut, M.; Kopka, K.; Eder, M., Improving the Imaging Contrast of ⁶⁸Ga-PSMA-11 by Targeted Linker Design: Charged Spacer Moieties Enhance the Pharmacokinetic Properties. Bioconjugate Chem. 2017, 28, 2485-2492; Kuo, H.-T.; Pan, J.; Zhang, Z.; Lau, J.; Merkens, H.; Zhang, C.; Colpo, N.; Lin, K.-S.; Benard, F., Effects of linker modification on tumor-to-kidney contrast of ⁶⁸Ga-labeled PSMA-targeted imaging probes. Mol Pharm. 2018, 15, 3502-3511; Benešová, M.; Bauder-Wüst, U.; Schäfer, M.; Klika, K. D.; Mier, W.; Haberkorn, U.; Kopka, K.; Eder, M., Linker Modification Strategies To Control the Prostate-Specific Membrane Antigen (PSMA)-Targeting and Pharmacokinetic Properties of DOTA-Conjugated PSMA Inhibitors. J. Med. Chem. 2016, 59, 1761-1775, each of which is incorporated by reference herein in its entirety). A person skilled in the art could develop and optimize a suitable linker for a particular application.

In some embodiments, a construct such as construct 120 is prepared by carrying out suitable reactions to couple targeting moiety 122 and chelator 126, for example via suitable chemical reaction, to yield an in vivo targeting construct 130, optionally with linker 124 interposing targeting moiety 122 and chelator 126. The radionuclide 128 is then added and bound to chelator 126, e.g. at a later time and in a hospital or clinic setting, to form the desired in vivo targeting metal chelate construct 120. In other embodiments, radionuclide 128 could be first chelated with chelator 126, and then chelator 126 is conjugated with targeting moiety 122 in any suitable manner to yield in vivo targeting chelate construct 120.

In some embodiments, the radionuclide 128 is bound to chelator 126 (including as part of construct 130) under mild temperature conditions, e.g. less than about 65° C., 60° C., 55° C., 50° C., 45° C., 40° C., 35° C. or 30° C. In some embodiments, the mild temperature conditions are between about 10° C. and 65° C., including any value or subrange therebetween, e.g. 15° C., 16° C., 17° C., 18° C., 19° C., 20° C., 21° C., 22° C., 23° C., 24° C., 25° C., 26° C., 27° C., 28° C., 29° C., 30° C., 35° C., 40° C., 45° C., 50° C., 55° C. or 60° C. In some embodiments, the radionuclide 128 is conjugated to chelator 126 or construct 130 at room temperature, i.e. in the range of about 15° C. to about 25° C., including any temperature value therebetween, e.g. 16° C., 17° C., 18° C., 19° C., 20° C., 21° C., 22° C., 23° C., or 24° C.

In some embodiments, the radionuclide 128 or construct 130 is combined with chelator 126 to form a metal chelate under mild pH conditions, e.g. between about 5.0 and about 7.4, including any value or subrange therebetween, e.g. 5.2, 5.4, 5.6, 5.8, 6.0, 6.2, 6.4, 6.6, 6.8, 7.0 or 7.2. In some embodiments the radionuclide 128 is conjugated to chelator 126 at approximately neutral pH, i.e. a pH of approximately 7.0, e.g. between about 6.8 and 7.2 including any value therebetween, e.g. 6.9, 7.0 or 7.1. In some embodiments, the radionuclide 128 is conjugated to chelator 126 at approximately physiological pH, i.e. at approximately pH 7.4, e.g. between about 7.2 and 7.6 including any value therebetween, e.g. 7.3, 7.4 or 7.5. In some embodiments, radionuclide 128 is combined with chelator 126 or construct 130 in aqueous solution. In some embodiments, the aqueous solution is free or substantially free of alcohol such as ethanol.

In some embodiments, the radionuclide 128 is combined with chelator 126 or construct 130 for an incubation period to allow a chelated metal complex to form. In some embodiments, the incubation period is between about 5 minutes and about 6 hours, including any period therebetween, e.g. 10, 15, 20, 25, 30, 45, 60 or 90 minutes, or 2, 3, 4 or 5 hours. In some embodiments, the incubation period is between about 5 minutes and about 30 minutes.

In some embodiments, the concentration of chelator 126 or construct 130 that is present when conjugated to radionuclide 128 is between about 10⁻⁴ to 10⁻⁷ M, including any value therebetween, e.g. 10⁻⁵ or 10⁻⁶ M. The concentration of chelator 126 or construct 130 that is used can be adjusted depending on the complexation kinetics between the particular chelator 126 and radionuclide 128 used in any particular embodiment. Similarly the temperature at which the radionuclide 128 is combined with chelator 126 or construct 130 can be varied depending on the complexation kinetics.

In some embodiments, in vivo radioisotope targeting chelate construct 120 is present in mammalian serum, optionally in human serum. In some embodiments, in vivo radioisotope targeting chelate construct 120 is stable in mammalian serum, optionally in human serum. In some embodiments, in vivo radioisotope targeting chelate construct 120 is present in mammalian serum within the body of a mammal, optionally in human serum within the body of the human. In some embodiments, in vivo radioisotope targeting chelate construct 120 is present in mammalian blood, optionally in human blood. In some embodiments, in vivo radioisotope targeting chelate construct 120 is present in mammalian blood within the body of a mammal, optionally in human blood in the body of the human. In some embodiments, in vivo radioisotope targeting chelate construct 120 is present within the body of a mammal, optionally the body of a human. In some embodiments, in vivo radioisotope targeting chelate construct 120 is present in a mammalian cell, optionally a human cell.

In some embodiments, radionuclide 128 is delivered to a selected location within the body of a mammalian subject by administering to the subject an in vivo radioisotope targeting chelate construct 120 incorporating the radionuclide 128 and a targeting moiety 122 that specifically directs the in vivo radioisotope targeting chelate construct 120, including the bound radionuclide 128, to the selected location within the body of the subject. In some embodiments, the method includes allowing the targeting moiety 122 to enhance the accumulation of the in vivo radioisotope targeting chelate construct 120 at the selected location within the body relative to other locations in the body to selectively deliver a dose of radiation to the selected location. In some embodiments, the in vivo radioisotope targeting chelate construct 120 is used to cause cell death at the selected location by delivering a targeted dose of radiation. In some embodiments, the cells that are killed at the selected location are cancer cells. In some embodiments, the radiation is alpha radiation; beta or gamma radiation could be used in other embodiments.

In some embodiments, in vivo radioisotope targeting chelate construct 120 is internalized by a cell within the mammalian subject, for example by endocytosis or otherwise. Thus in some embodiments, in vivo radioisotope targeting chelate construct 120 is present within a mammalian cell. In some embodiments, the in vivo radioisotope targeting chelate construct 120 is present within a human cell.

In some embodiments, the in vivo radioisotope targeting chelate construct 120 is prepared prior to administration of construct 120 to a subject by combining an in vivo radioisotope targeting construct 130 having a targeting moiety 122, a chelator 126 and optionally a linker 124 with a radionuclide 128 to form the in vivo radioisotope targeting chelate construct 120. In some embodiments, the combining is carried out at a mild temperature, e.g. at a temperature in the range of about 10° C. to about 65° C., including any value therebetween e.g. 15° C., 16° C., 17° C., 18° C., 19° C., 20° C., 21° C., 22° C., 23° C., 24° C., 25° C., 26° C., 27° C., 28° C., 29° C., 30° C., 35° C., 40° C., 45° C., 50° C., 55° C. or 60° C. In some embodiments, the combining is carried out at a mild pH, e.g. an approximately neutral pH or an approximately physiological pH. In some embodiments, the mild pH is a pH of between about 5.0 and about 7.4, including any value therebetween e.g. 5.2, 5.4, 5.6, 5.8, 6.0, 6.2, 6.4, 6.6, 6.8, 7.0 or 7.4. In some embodiments, the mild pH is approximately 6.0. In some embodiments, the combining is carried out a physiological pH, e.g. in the range of about. 7.0 to 7.4 including any value therebetween, e.g. 7.1, 7.2 or 7.3. In some embodiments, radionuclide 128 is combined with in vivo radioisotope targeting construct 130 in aqueous solution. In some embodiments, the aqueous solution is free or substantially free of alcohols such as ethanol. In some embodiments, the combining is carried out for a period of between about 5 and about 30 minutes, including any value therebetween e.g. 10, 15, 20 or 25 minutes.

In some embodiments, in vivo targeting chelate construct 120 is used in diagnostic applications. For example, in vivo targeting chelate construct 120 may be administered to a subject in any suitable manner, and any suitable imaging technology or procedure may be used to evaluate the localization of the targeting chelate construct 120 within the body via targeting moiety 122 by visualizing the location of bound radionuclide 128, e.g. positron emission tomography (PET) imaging or single-photon emission computerized tomography (SPECT) imaging. Such imaging procedures can be carried out for example to diagnose a subject as having a particular disorder or type of cancer, or to localize regions of the subject's body affected by the particular disorder or type of cancer. In some embodiments, localization of targeting chelate construct 120 to a target organ, region or plurality of loci within the body as evaluated by such imaging technology may be indicative that the subject has a particular form of cancer, and/or can be used to evaluate the extent of the cancer and or locations within the body wherein cancerous cells are or may be located, and/or can be used to evaluate the extent of metastasis of the cancer.

In some embodiments, constructs such as targeting chelate construct 120 are used in therapeutic applications, for example to carry out targeted radionuclide therapy. For example, targeting chelate construct 120 may be administered to a subject in any suitable manner, and the targeting effect imparted by targeting moiety 122 can be used to deliver the chelated radionuclide 128 to a desired location within the subject's body. In some embodiments, radiation from radionuclide 128 is used to kill cells at the desired location. In some embodiments, the cells that are killed at the desired location are cancer cells. In some embodiments, targeting construct 120 is used to perform targeted radionuclide therapy. In some embodiments, targeting construct 120 is used to perform targeted alpha therapy.

In some embodiments, a pharmaceutical composition is provided, the pharmaceutical composition comprising a construct such as targeting construct 120 and a pharmaceutically acceptable carrier. The pharmaceutical composition may include any suitable excipient, vehicle, buffer, diluent, binder, thickener, lubricant, preservative or the like, and may be provided in any desired state, e.g. as a liquid, suspension, emulsion, paste, or the like. In some embodiments, the pharmaceutical composition can be administered in any suitable manner, e.g. orally, intravenously, intramuscularly, subcutaneously, intraperitoneally, intratumorally, by inhalation, or the like.

In some embodiments, a method of prophylaxis and/or treatment of a subject having or believed to have cancer is provided. In some embodiments, the method comprises administering an in vivo targeting chelate construct 120 or a pharmaceutical composition comprising such a targeting chelate construct 120 to the subject. In some embodiments, the method comprises administering a therapeutically and/or prophylactically effective amount of the targeting chelate construct 120 to the subject.

In some embodiments, the subject is a mammal. In some embodiments, the subject is a human. In alternative embodiments, the subject is livestock or a pet, e.g. a horse, cow, sheep, goat, cat, dog, rabbit, or the like. In some embodiments, the subject is a monkey.

While exemplary embodiments are described herein with reference to the targeting and killing of cancer cells, such constructs can be used for the selective killing and/or ablation of other undesired cell types, for example bacteria, fungi, cells implicated in autoimmune disorders, virus-infected cells, parasites, and so on.

In some embodiments, the metals that can be used as metal 128 include actinides, lanthanides, rare earth metals, or main group metals. In some embodiments, the lanthanide is La, Ce, Pr, Nd, Pm, Sm, Eu, Gd, Tb, Dy, Ho, Er, Tm, Yb or Lu. In some embodiments, the lanthanide is Gd, Lu, Pr, Nd, Ho, Er or Yb. In some embodiments, the lanthanide is a radiolanthanide. In some embodiments, the actinide is Ac, Th, Pa, U, Np, Pu, Am, Cm, Bk, Cf, Es, Fm, Md, No or Lr. In some embodiments, the actinide is Ac, Th or U. In some embodiments, the actinide is a radioactinide. In some embodiments, the rare earth metal is Sc, Y, La, Ce, Pr, Nd, Pm, Sm, Gd, Tb, Dy, Ho, Er, Tm, Yb or Lu. In some embodiments, the metal is a trivalent lanthanide ion.

In some embodiments, the metal is a radioisotope. In some embodiments, the radioisotope is any desired radioisotope, e.g. ²²⁵Ac, ²²⁷Th, ²²⁶Th, ²¹¹At, ⁴⁴Sc, ⁹⁰Y, ⁸⁹Zr, ¹⁷⁷Lu, ¹¹¹In, ^{86/89/90}Y, ²¹¹At, ²¹¹Fr, ^{212/213}Bi, ¹⁵³Sm, ^{161/166}Ho, ^{165/166}Dy, ^{161/155}Tb, ¹⁴⁰La, ^{142/143/145}Pr, ¹⁵⁹Gd, ^{169/175}Yb, ^{167/170}Tm, ¹⁶⁹Er, ¹⁴⁹Pm, ¹⁵⁰Eu, ⁶⁸Ga, ¹³⁷Cs, ¹⁴¹Ce, or the like.

In some embodiments, the metal is actinium (Ac), lutetium (Lu), bismuth (Bi), gallium (Ga), indium (In), terbium (Tb), thorium (Th), or Caesium (Cs). In some embodiments, the metal is actinium (III) (Ac³⁺), lutetium (III) (Lu³⁺), bismuth (III) (Bi³⁺), gallium (III) (Ga³⁺), indium (III) (In³⁺), terbium (Tb³⁺), thorium (III) (Th³⁺), or Cesium (I) (Cs¹⁺). In some embodiments, the metal is ²²⁵Ac, ¹⁷⁷Lu, ²¹³Bi, ²³²Th, ²³⁰Th, ²²⁸Th, ⁶⁸Ga, ¹⁶¹Tb, ¹⁵⁵Tb, ¹⁵²Tb, ¹⁴⁹Tb, ¹¹¹In, or ¹³⁷Cs. In some embodiments, the metal is ²²⁷Th, ²²⁵Ac, ¹⁵⁵Tb, ¹⁷⁷Lu, ¹¹¹In, ¹³²La, ²³⁵La, ⁹⁰Y ⁶⁸Ga, ⁴⁴Sc, ²⁰³Pb, or ²¹²Pb.

In some embodiments, H₄noneunpaX is bound to a metal ion to form a coordination complex. In some embodiments, the coordination complex is referred to as a metal chelate. In some embodiments, the metal chelate or H₄noneunpaX as the chelating ligand is associated with one or more cations as counter ions, for example Na⁺, K⁺, Ca²⁺ or the like. In some embodiments, the metal chelate or the chelating ligand is fully protonated. In some embodiments, the metal chelate or the chelating ligand is in its free acid form. In some embodiments, the metal chelate or the chelating ligand is in a partially protonated state.

In some embodiments, the coordination complex is present in mammalian serum, optionally human serum. In some embodiments, the coordination complex is stable in mammalian serum, optionally human serum. In some embodiments, the coordination complex is present in mammalian serum within the body of the mammal, optionally present in human serum within the body of the human. In some embodiments, the coordination complex is present in blood, optionally human blood. In some embodiments, the coordination complex is stable in mammalian blood, optionally human blood. In some embodiments, the coordination complex is present in mammalian blood within the body of the mammal, optionally present in human blood within the body of the human. In some embodiments, the coordination complex is present within the body of a mammal, optionally present within the body of a human. In some embodiments, the coordination complex is present within a cell of a mammalian subject, optionally present within a cell of a human subject.

### EXAMPLES

Further embodiments are described with reference to the following examples, which are intended to be illustrative and not limiting in nature.

### Example 1.0—Synthesis and Characterisation of H₄noneunpaX

The nonadentate chelating ligand, H₄noneunpa, has been previously reported^{8,13} and was synthesised following established procedures.⁸ Preparation of H₄noneunpaX was carried out in accordance with the linear synthetic route outlined in Scheme 1.

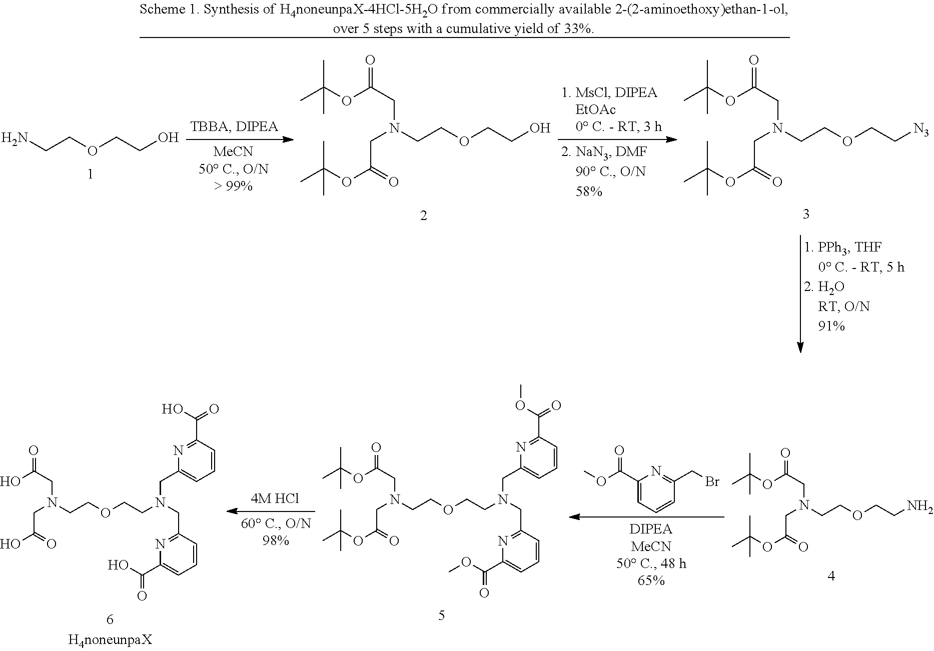

The synthesis of H₄noneunpaX proceeds through N-alkylation of commercially available 2-(2-aminoethoxy)ethan-1-ol (1) with tert-butyl bromoacetate (toba) under mild conditions to give di-tert-butyl ester (2) in high yields. Sequential mesylation of compound (2) followed by displacement with sodium azide, proceeded cleanly to give the corresponding azide (3) in high yields. Diisopropylethylamine (dipea) was selected an appropriate base for the mesylation of compound (2) in order to precipitate the HCl equivalent generated in the reaction, and thus minimise formation of the corresponding chloride side product. Staudinger reduction of azide (3) was employed to generate the corresponding primary amine (4) which could be used directly without additional purification. Subsequent N-alkylation of amine (4) with two equivalents of methyl 6-(bromomethyl)picolinate afforded the protected ligand (5) in modest yields. DIPEA was found to be a superior base over K₂CO₃ in this reaction. Attempts to isolate compound (5) while using K₂CO₃ as a base were hampered by difficulties in purification, presumably due to the product chelating free K⁺ ions from solution. This difficulty was mitigated by using an organic base and generated the product in high yield and high purity. Final deprotection of compound (5) was achieved through ester hydrolysis in 4 M HCl, followed by reverse phase high performance liquid chromatography (HPLC). Co-evaporation of the purified ligand with 3 M HCl was then used to afford H₄noneunpaX (6) as a HCl salt.

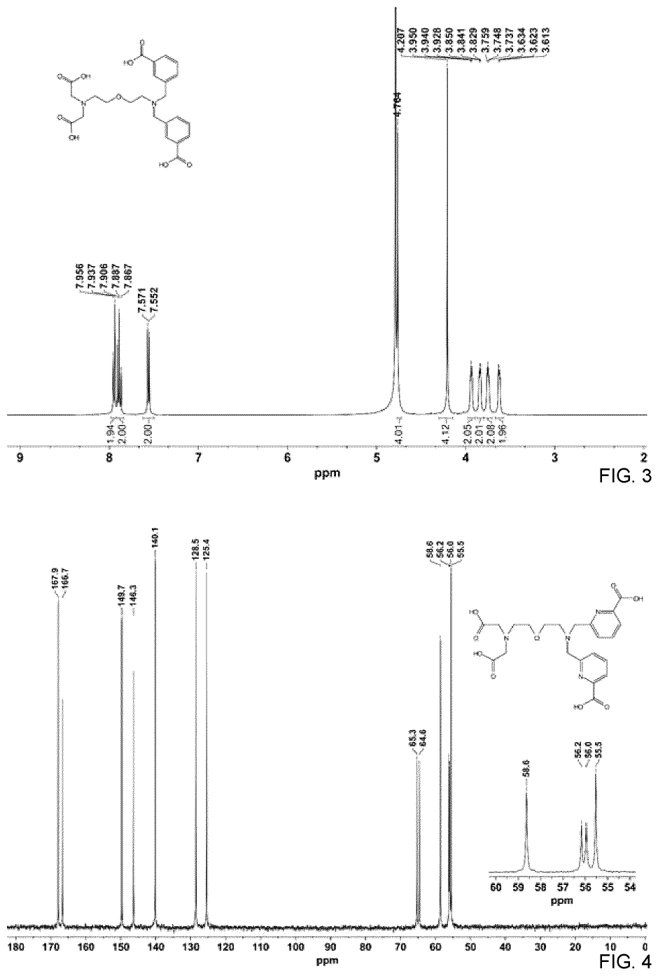

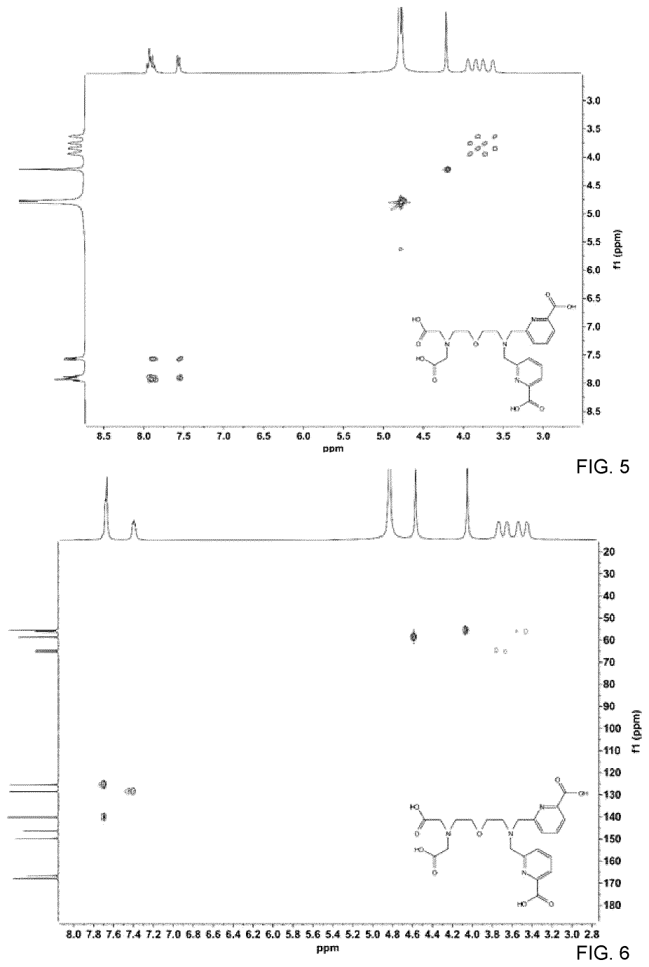

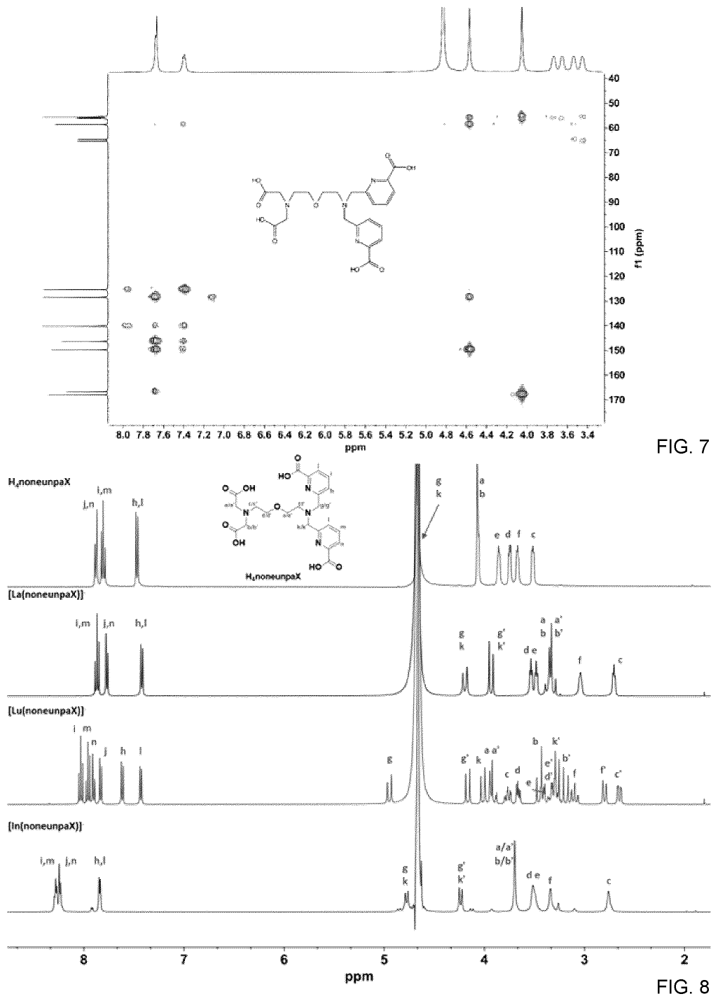

The purified ligand and all synthetic intermediates were fully characterised by NMR spectroscopy (¹H, ¹³C{¹H}, COSY, HSQC) and mass spectrometry (LR/HR-MS). Elemental analysis (EA) and HPLC of the final ligand was performed in order to confirm the purity of the isolated product. Results for H₄noneunpaX are shown in FIGS. 3-7.

Di-tert-butyl 2,2′-((2-(2-hydroxyethoxy)ethyl)azanediyl)diacetate (2): tert-Butyl bromoacetate (4.30 mL, 5.57 g, 28.6 mmol) was added slowly to a solution of 2-(2-aminoethyoxyl)ethan-1-ol (1) (1.53 g, 14.3 mmol) and diisoproylethylamine (5.00 mL, 3.69 g, 28.6 mmol) dissolved in dry MeCN (150 mL). The reaction mixture was heated to 50° C. and stirred overnight. Upon completion, the volatiles were removed in vacuo and the resulting residue re-dissolved in EtOAc (150 mL). After standing for 10 min at RT, the white precipitate formed was removed via vacuum filtration, washed with cool EtOAc (50 mL) and discarded. The filtrate was washed with de-ionised water (3×150 mL) and the combined aqueous phase back extracted with EtOAc (2×150 mL). The combined organics were evaporated in vacuo to yield the title compound as a pale-yellow oil (4.69 g, 99%). ¹H NMR (300 MHz, CDCl₃, 298 K) 3.66 (2H, t, J=4.6 Hz, 1-CH₂), 3.60 (2H, t, J=5.2 Hz, 3-CH₂), 3.52 (2H, t, J=4.6 Hz, 2-CH₂), 3.47 (4H, s, 5-CH₂), 2.96 (1H, br s, 7-OH), 2.92 (2H, t, J=5.2 Hz, 4-CH₂), 1.42 (18H, s, 6-C(CH₃)₃). ¹³C {¹H} NMR (75 MHz, CDCl₃, 298 K) 171.0 (6-C), 81.2 (7-C), 72.4 (2-C), 70.2 (3-C), 61.9 (1-C), 56.8 (5-C), 53.4 (4-C), 28.3 (8-C). ESI-MS (MeOH) 334.26 [M+H]⁺.

Di-tert-butyl 2,2′-((2-(2-azidoethoxy)ethyl)azanediyl)diacetate (3):

Methanesulphonyl chloride (1.34 mL, 1.98 g, 17.3 mmol) was added slowly to a solution of di-tert-butyl 2,2′-((2-(2-hydroxyethoxy)ethyl)azanediyl)diacetate (2) (5.26 g, 15.7 mmol) and diisoproylethylamine (3.01 mL, 2.24 g, 17.3 mmol) in EtOAc (15 mL) at 0° C. After 10 minutes, the suspension was warmed to RT and stirred for a further 3 h. Upon completion, the suspension was cooled to 0° C. and the white solid separated by vacuum filtration. The filtrate was diluted with EtOAc (50 mL) and washed with de-ionised H₂O (3×50 mL). The organic phase was dried over Na₂SO₄ and evaporated in vacuo to yield the corresponding mesylate as a pale-yellow oil which was used without further purification.

NaN₃ (3.08 mg, 47.2 mmol) was added to a solution of crude mesylate dissolved in dry DMF (20 mL) and the suspension heated at 80° C. O/N. Upon completion, the solution was cooled to RT and diluted with de-ionised H₂O (50 mL). The aqueous phase was extracted with DCM (3×50 mL) and the combined organics washed with 5% LiCl aq. sol. (50 mL). The volatiles were removed in vacuo and the resulting residue purified via silica gel chromatography (Combiflash automated purification system; A: DCM, B: MeOH; 100% to 5% B). The title compound was attained as a pale-yellow oil (3.25 g, 58%). ¹H NMR (300 MHz, CDCl₃, 298 K) 3.62-3.57 (4H, m, 2- and 3-CH₂), 3.46 (4H, s, 5-CH₂), 3.33 (2H, t, J=5.0 Hz, 1-CH₂), 2.92 (2H, t, J=5.6 Hz, 4-CH₂), 1.42 (18H, s, 6-(C(CH₃)₃). ¹³C {1H} NMR (75 MHz, CDCl₃, 298 K) 170.9 (6-C), 81.0 (7-C), 70.9 (3-C), 69.8 (2-C), 56.9 (5-C), 53.6 (4-C, 50.8 (1-C), 28.3 (8-C). ESI-MS (MeOH) 359.21 [M+H]⁺.

Di-tert-butyl 2,2′-((2-(2-aminoethoxy)ethyl)azanediyl) diacetate (4): Di-tert-butyl 2,2′-((2-(2-aminoethoxy)ethyl)azanediyl) diacetate (3) (2.81 g, 7.85 mmol) was dissolved in dry THE (30 mL) and cooled to 0° C. PPh₃ (2.47 mg, 9.42 mmol) was added slowly to the reaction mixture under Ar, and the resulting solution allowed to warm to RT and stirred for 5 h. The resulting solution was then added dropwise into de-ionised H₂O (350 mL) and the suspension stirred overnight. The THE was removed in vacuo and the off-white suspension left to precipitate at RT for 1 h. The white precipitate was removed via vacuum filtration, and the aqueous phase concentrated to ˜100 mL in vacuo. The aqueous phase was extracted with CH₂Cl₂ (4×75 mL) and the combined organic phase evaporated in vacuo to give the title compound as a pale-yellow oil (2.38 g, 91%). ¹H NMR (300 MHz, CDCl₃, 298 K) 6.72 (3H, br s, 7-NH₂), 3.71 (2H, t, J=4.5 Hz, 2-CH₂), 3.58 (2H, t, J=5.1 Hz, 3-CH₂), 3.10 (2H, t, J=4.5 Hz, 1-CH₂), 2.91 (2H, t, J=5.1 Hz, 4-CH₂), 1.42 (18H, s, 6-(C(CH₃)₃). ¹³C {¹H} NMR (75 MHz, CDCl₃, 298 K) 171.0 (6-C), 81.6 (7-C), 68.8 (3-C), 67.9 (2-C), 56.2 (5-C), 53.3 (4-C), 40.5 (1-C), 28.3 (8-C). ESI-MS (MeOH) 333.19 [M+H]⁺.

(^{t}Bu)₂(Me)₂noneunpaX (5). Methyl (6-bromomethyl) picolinate (515 mg, 2.824 mmol) was added to a solution of di-tert-butyl 2,2′-((2-(2-aminoethoxy)ethyl)azanediyl)diacetate (4) (372 mg, 1.12 mmol) in dry MeCN (15 mL) under argon. Diisoproylethylamine (585 μL, 434 mg, 3.36 mmol) was added and the resulting pale-yellow solution stirred for 1 h at ambient temperature. The reaction mixture was subsequently heated to 50° C. and stirred overnight. Upon completion, the volatiles were removed in vacuo and the resulting residue was re-dissolved in CH₂Cl₂ (25 mL). The organic phase was washed with de-ionised H₂O (3×25 mL) and brine (25 mL), dried over Na₂SO₄ and evaporated in vacuo. The crude residue was purified via flash column chromatography on neutral alumina (Combiflash automated purification system; A: CH₂Cl₂, B: MeOH; 100% A to 5% B). The product was attained as a pale-yellow oil (459 mg, 65%). ¹H NMR (300 MHz, CDCl₃,298 K) 7.90 (2H, dd, ³J=7.3 Hz, ⁴J=1.1 Hz, 10-CH), 7.80 (2H, dd, ³J=8.1 Hz, ⁴J=1.4 Hz, 8-CH), 7.75 (2H, t, ³J=7.5 Hz, 9-CH), 3.94 (4H, s, 7-CH₂), 3.90 (6H, s, 11-CH₃), 3.49 (2H, t, ³J=5.6 Hz, 2-CH₂), 3.44 (2H, t, ³J=5.8 Hz, 3-CH₂), 3.40 (4H, s, 5-CH₂), 2.84 (2H, t, ³J=5.8 Hz, 4-CH₂), 2.74 (2H, t, ³J=5.6 Hz, 1-CH₂), 1.35 (18H, s, 6-C(CH₃)₃). ¹³C {¹H} NMR (75 MHz, CDCl₃, 298 K) 170.8 (8-C), 165.9 (15-C), 160.8 (14-C), 147.3 (10-C), 137.5 (12-C), 126.1 (11-C), 123.6 (13-C), 80.9 (7-C), 70.5 (3-C), 69.4 (2-C), 60.9 (9-C), 56.7 (5-C), 54.1 (1-C), 53.6 (4-C), 52.9 (16-C), 28.2 (8-C). ESI-MS (MeOH) 653.3 [M+Na]⁺.

H₄noneunpaX·4HCl·5H₂O (6). (^{t}Bu)₂(Me)₂noneunpaX (5) (168 mg, 0.257 mmol) was dissolved in 4 M HCl (5 mL) and heated at 60° C. overnight. The volatiles were removed in vacuo and the resulting off-white solid purified via RP-HPLC (A: H₂O (0.1% TFA), B: MeCN; 100% A to 20% B, 30 min., t_{R}˜ 21.5 min). Pure H₄noneunpaX was attained as a white HCl salt (146 mg, 87%) by co-evaporation with 3 M HCl. ¹H NMR (400 MHz, D₂O, 298 K) 7.95 (2H, d, ³J=7.7 Hz, 1-CH), 7.89 (2H, t, ³J=7.7 Hz, 2-CH), 7.57 (2H, d, ³J=7.7 Hz, 3-CH), 4.76 (4H, s, 3-CH₂), 4.21 (4H, s, 9-CH₂), 3.94 (2H, t, ³J=4.4 Hz, 6-CH₂), 3.84 (2H, t, ³J=4.4 Hz, 7-CH₂), 3.75 (2H, t, ³J=4.4 Hz, 5-CH₂), 3.63 (2H, t, ³J=4.4 Hz, 8-CH₂). ¹³C {¹H} NMR (75 MHz, CDCl₃,298 K) 167.9 (13-C), 166.7 (1-C), 149.7 (6-C), 146.3 (2-C), 140.1 (3-C), 128.5 (5-C), 125.4 (4-C), 65.3 (10-C), 64.6 (9-C), 58.6 (7-C), 56.2 (11-C), 56.0 (8-C), 55.5 (12-C). ESI-MS (H₂O) 491.1 [M+H]⁺. HR-ESI-MS (H₂O) calcd. for [C₂₂H₂₆N₄O₉₊H]⁺: 491.1700; found [M+H]⁺: 491.1779. Elemental analysis: calcd. % for H₄noneunpaX·4HCl·5H₂O (C₂₂H₂₆N₄O₉.4HCl·5H₂O=726.178 gmol⁻¹): C 37.59, H 5.36, N 7.97; found: C 37.65, H 5.43, N 7.97.

### Example 2.0—Metal Complexation and NMR Characterisation

The coordination characteristics of H₄noneunpaX were investigated through complexation of a series of non-radioactive trivalent metal ions (La³⁺, Lu³⁺ and In³⁺) and assessment of the changes in spectral features as a function of metal ionic radius using NMR spectroscopy (¹H, COSY). Metal complexes of H₄noneunpaX were prepared by mixing equimolar quantities of the ligand with the appropriate metal salts in D₂O and adjustment of the pD to neutral using NaOD (0.1 M). The corresponding solutions were then filtered, and analysed directly via H NMR spectroscopy, FIG. 8. Further confirmation of metal complexation was achieved using high resolution electrospray ionisation mass spectrometry (HR-ESI-MS), wherein each metal complex was confirmed by the presence of a monocationic peak corresponding to the [M+2H]⁺ species, Table 2.

TABLE 2
High-resolution electrospray ionisation mass spectrometry
characterisation of metal complexes of H₄noneunpaX with La³⁺,
Lu³⁺ and In³⁺.

| | Theoretical Mass | Experimental Mass |
|---|---|---|
| Metal Complex | [M + 2H]⁺ | [M + 2H]⁺ |
| [La(noneunpaX)]- | 627.0536 | 627.0600 |
| [Lu(noneunpaX)]- | 663.0873 | 663.0946 |
| [In(noneunpaX)]- | 603.0506 | 603.0572 |

Full ¹H NMR characterisation data (¹H, COSY) was obtained.

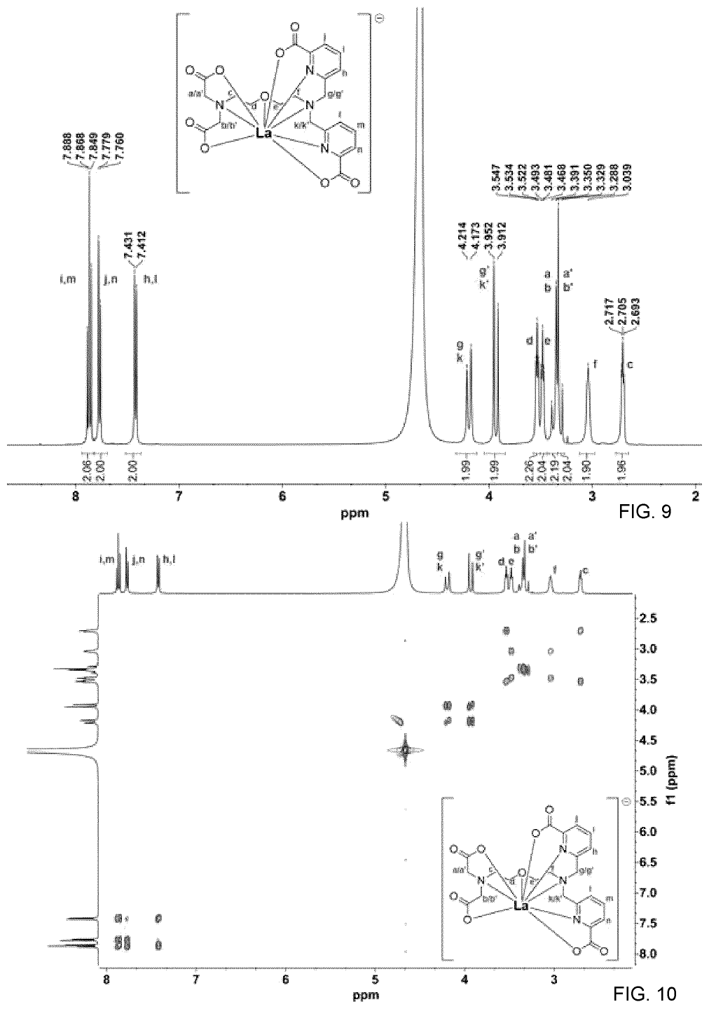

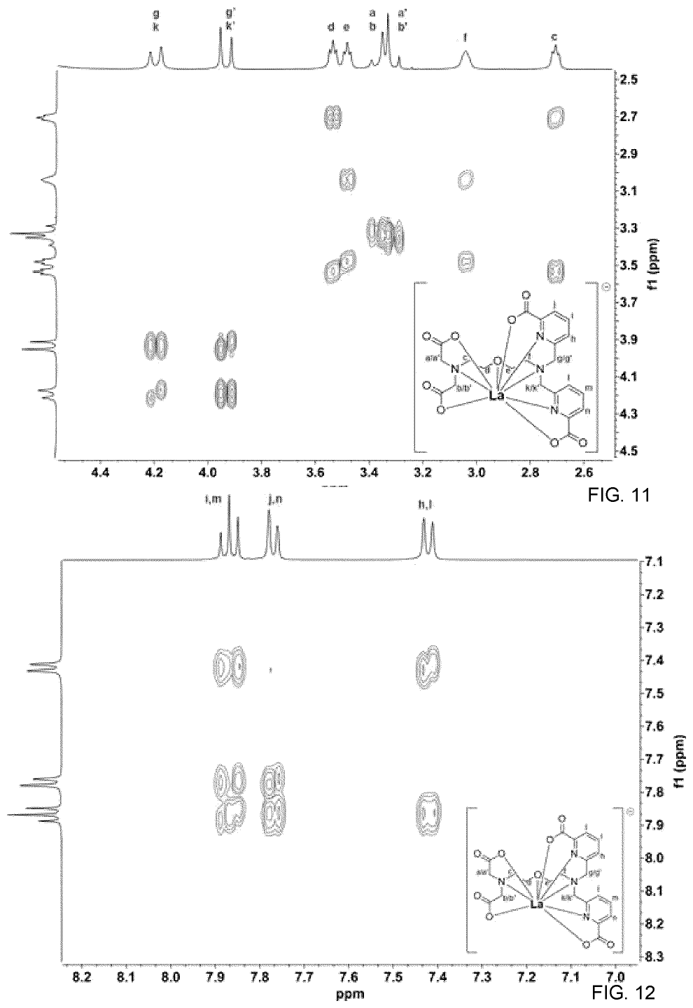

Na[La(noneunpaX)]. Results shown in FIGS. 9-12. La(NO₃)₃·6H₂O (2.9 mg, 6.75 μmol, 1.0 equiv.) was added directly to a solution of H₄noneunpaX·4HCl·5H₂O (4.9 mg, 6.75 μmol, 1.0 equiv.) in D₂O (300 μL). The solution was mixed thoroughly at 1000 rpm using a vortex mixer, and the pD adjusted to ˜7.0 using dilute NaOD solution to give the corresponding [La(noneunpaX)]⁻ complex. The white precipitate was filtered, and the filtrate characterized directly without additional purification. ¹H NMR (400 MHz, D₂O, 298 K, pD 7.0) 7.87 (2H, t, ³J=7.7 Hz, i- and m-CH), 7.77 (2H, d, ³J=7.5 Hz, j- and n-CH), 7.42 (2H, d, ³J=7.7 Hz, h- and 1-CH), 4.19 (2H, d, ²J=16.4 Hz, g- and k-CH), 3.93 (2H, d, ²J=16.4 Hz, g′- and k′-CH), 3.53 (2H, t, ³J=5.0 Hz, d-CH₂), 3.48 (2H, d, ³J=4.9 Hz, e CH₂), 3.37 (2H, d, ²J=16.3 Hz, a- and b-CH), 3.31 (2H, d, ²J=16.3 Hz, a′- and b′-CH), 3.04 (2H, br t, f-CH₂), 2.71 (2H, t, ³J=5.0 Hz, c-CH₂). LR-ESI-MS (H₂O) 627.0 [M+2H]⁺, 624.8 [M]⁻. HR-ESI-MS (H₂O) calcd. for [C₂₂H₂₂LaN₄O₉₊2H]⁺: 627.0536; found [M+2H]⁺: 627.0600.

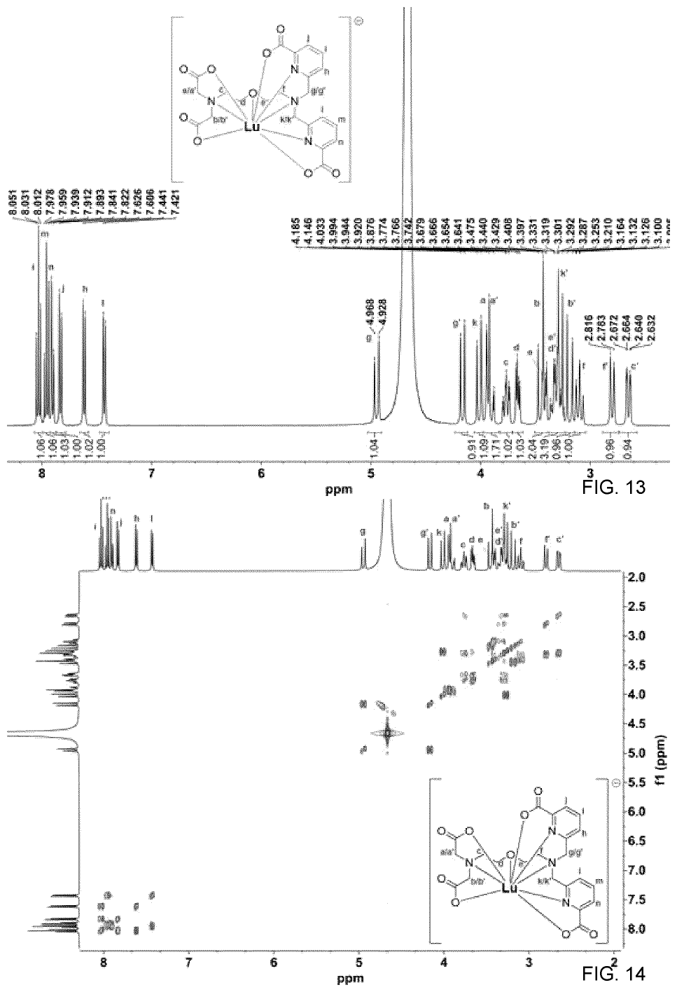

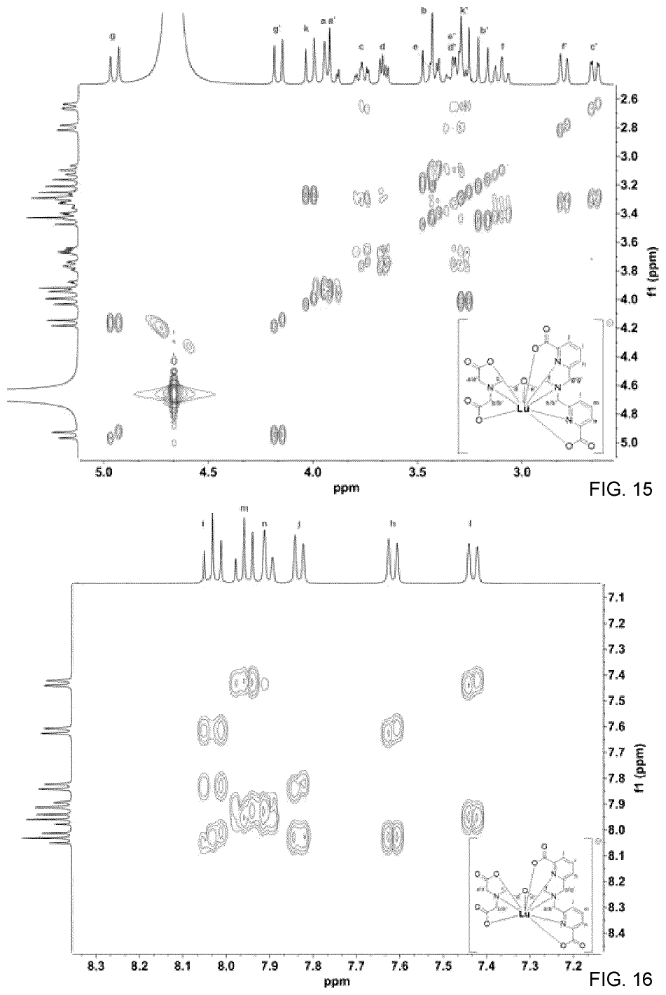

Na[Lu(noneunpaX)]. Results shown in FIGS. 13-16. Lu(NO₃)₃·H₂O (2.4 mg, 6.75 μmol, 1.0 equiv.) was added directly to a solution of H₄noneunpaX·4HCl·5H₂O (4.9 mg, 6.75 μmol, 1.0 equiv.) in D₂O (300 μL). The solution was mixed thoroughly at 1000 rpm using a vortex mixer, and the pD adjusted to ˜6.5 using dilute NaOD solution to give the corresponding [Lu(noneunpaX)]⁻ complex. The solution was filtered and analyzed without additional purification. ¹H NMR (400 MHz, D₂O, 298 K, pD 6.5) 8.03 (1H, t, ³J=7.8 Hz, i-CH), 7.96 (1H, t, ³J=7.7 Hz, m-CH), 7.90 (1H, d, ³J=7.7 Hz, n-CH), 7.83 (1H, d, ³J=7.8 Hz, j-CH), 7.61 (1H, d, ³J=7.8 Hz, h-CH), 7.43 (1H, d, ³J=7.7 Hz, I-CH), 4.95 (1H, d, ²J=15.8 Hz, g-CH), 4.17 (1H, d, ²J=15.8 Hz, g′-CH), 4.01 (1H, d, ²J=15.6 Hz, k-CH), 3.97 (1H, d, ²J=17.4 Hz, a-CH), 3.90 (1H, d, ²J=17.4 Hz, a′-CH), 3.77 (1H, ddd, c-CH), 3.66 (1H, dd, d-CH), 3.45 (1H, d, ²J=18.4 Hz, b-CH), 3.43-3.40 (1H, m, e-CH), 3.36-3.24 (2H, m, e′- and d′-CH), 3.27 (1H, d, ²J=15.6 Hz, k′-CH), 3.19 (1H, d, ²J=18.4 Hz, b′-CH), 3.10 (1H, ddd, f-CH), 2.80 (1H, d, ²J=12.9 Hz, f′-CH), 2.65 (1H, dd, c′-CH). LR-ESI-MS (H₂O) 663.1 [M+2H]⁺, 661.1 [M]⁻. HR-ESI-MS (H₂O) calcd. for [C₂₂H₂₂LuN₄O₉₊2H]⁺: 663.0873; found [M+2H]⁺: 663.0946.

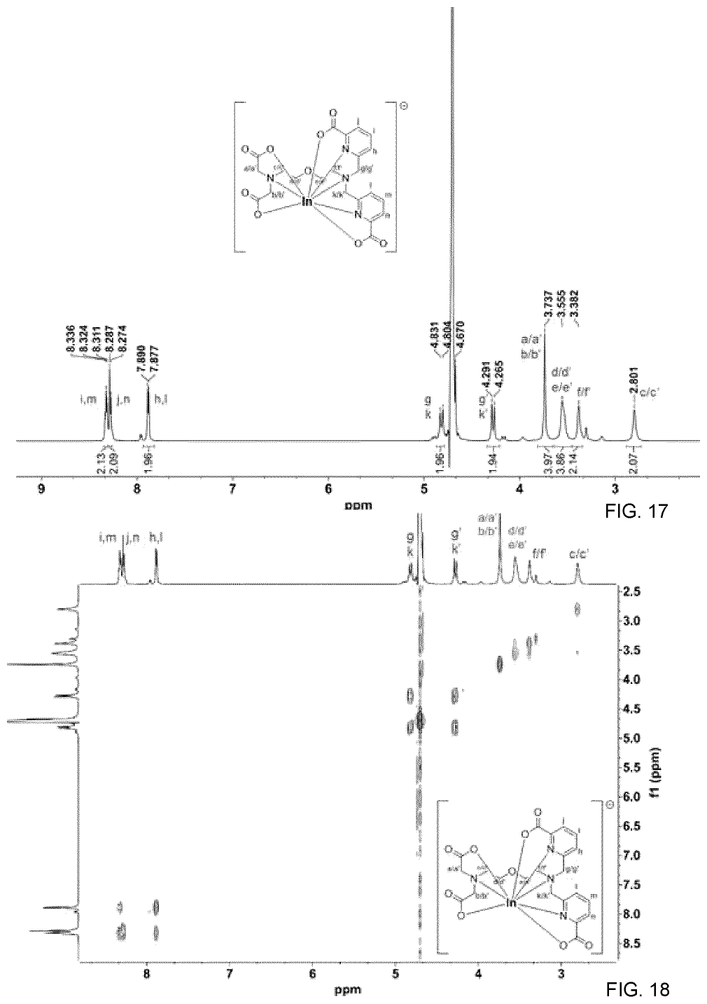

Na[In(noneunpaX)]. Results shown in FIGS. 17 and 18. In(NO₃)₃·H₂O (2.1 mg, 6.75 μmol, 1.0 equiv.) was added directly to a solution of H₄noneunpaX·4HCl·5H₂O (4.9 mg, 6.75 μmol, 1.0 equiv.) in D₂O (300 μL). The solution was mixed thoroughly at 1000 rpm using a vortex mixer, and the pD adjusted to ˜4.5 using dilute NaOD solution to give the corresponding [In(noneunpaX)]⁻ complex. The solution was filtered and analyzed without additional purification. ¹H NMR (400 MHz, D₂O, 298 K, pD 5.5) 8.32 (2H, t, ³J=5.2 Hz, i- and m-CH), 8.28 (2H, d, ³J=5.0 Hz, j- and n-CH), 7.88 (2H, d, ³J=5.0 Hz, h- and I-CH), 4.82 (2H, d, ²J=10.7 Hz, g and k-CH₂), 4.28 (2H, d, ²J=10.7 Hz, g′- and k′-CH₂), 3.74 (4H, s, a/a′- and b/b′-CH₂), 3.56 (4H, br s, d/d′- and e/e′-CH₂), 3.38 (2H, br s, f/f′ CH₂), 2.80 (2H, br s, c/c′-CH₂). LR-ESI-MS (H₂O) 603.0 [M+2H]⁺, 625.0 [M+H+Na]⁺. HR-ESI-MS (H₂O) calcd. for [C₂₂H₂₂InN₄O₉₊2H]⁺: 603.0506; found [M+2H]⁺: 603.0572.

The ¹H NMR spectrum of [La(noneunpaX)]⁻ shows the formation of a single, symmetric isomer in solution, with characteristic diastereotopic splitting for the methylenic protons associated with the four pendent donor arms of the ligand, implying coordination of all four donor groups to the metal centre. Sharply resolved resonances are observed for the protons within the ethylene bridged backbone which indicates the formation of a metal complex with the central ethereal oxygen coordinated. In contrast, the NMR spectra for the Lu³⁺ complex reveal a single, asymmetric isomer, wherein chemically distinct resonances for each picolinic acid donor arm are seen in the aromatic region. Notably, one picolinate donor arm (Hₗ, Hₘ, Hₙ) maintains the same chemical shift as seen in the La³⁺ complex, while the second picolinate arm (Hₕ, Hᵢ, Hⱼ) exhibits a significant downfield shift, indicating closer coordination to the Lu³⁺ metal centre and a change in ligand conformation at this position. This aspect is further observed for the adjacent methylenic protons (H_{g} and H_{g′}) which exhibit distinctive diastereotopic splitting, with characteristically large coupling constants (²J_{AB}=15.8 Hz), and appear in two distinct chemical environments (Δδ_{g/g′}, =0.78 ppm). The remaining methylenic protons associated with the pendent donor arms all also become inequivalent on metal ion complexation, appearing as diastereotopic doublets, which can be clearly distinguished in the ¹H-¹H COSY NMR spectrum, FIGS. 14-16. In contrast to [La(noneunpaX)]⁻, the Lu³⁺ complex shows further splitting for all eight protons in the backbone, with large differences in chemical shift for mutually coupled protons (e.g. H_{c}, and H_{c′},) which suggests a rigid complexation environment and close coordination to the metal centre.

The metal complexes of H₄noneunpaX share similar spectral characteristics to those reported for H₄noneunpa; whereby fully saturated metal coordination spheres were achieved with La³⁺ and Lu³⁺ ions, as indicated by diastereotopic splitting of the pendent donor arm resonances and backbone ethylene bridges. Interestingly, the opposite trend in metal complex symmetry was attained for H₄noneunpa, wherein the [La(noneunpa)]⁻ complex appears as an asymmetric isomer, while the [Lu(noneunpa)]⁻ complex is fully symmetric.⁸

Analysis of the [In(noneunpaX)]⁻ complex using NMR spectroscopy was hampered by low solubility at neutral and basic pH, therefore the ¹H NMR spectrum in FIG. 8 was recorded under acidic conditions (pH˜4.0). The spectrum obtained shows the formation a different symmetric isomer in which both picolinate donors are coordinated to the metal centre, while the two acetate donor arms remain unbound, as indicated by the presence of a 4H singlet resonance at 3.50 ppm corresponding to H_{a/a′} and H_{b/b′}. This assignment is also supported by the resonances observed for the backbone methylene protons, which appear as broad signals in the NMR spectrum. At higher pH values (pH˜5.0-8.5) a second species was observed, which may correspond to the fully coordinated complex, however a complete spectral analysis was not possible.

### Example 3.0—Solution Thermodynamic Stability Studies and Complexation Equilibria with Sc³⁺, In³⁺, Lu³⁺, Dy³⁺, Gd³⁺, Sm³⁺, La³⁺

Prior to investigation of the thermodynamic stability of different metal complexes, the protonation constants of the chelating ligand must be determined independently, due to the competition between a given metal ion with a proton for the same coordinating group in the metal complex reaction. Combined potentiometric and UV-vis spectrophotometric titrations were used to evaluate the protonation constants of H₄noneunpaX between pH˜2-11.5, while acidic in-batch UV-vis spectrophotometric titrations were used to determine the protonation constants for the most acidic protons, which were below the threshold of the pH electrode (pH<2). All eight protonation constants for H₄noneunpaX were determined through refinement of the experimental data using HyperSpec²⁴ and Hyperquad²⁵, Table 3.

TABLE 3
Protonation constants (log K_{HqL})^{a} of H₄noneunpaX
at 25° C. and I = 0.16M NaCl.

| Reaction | log β | log K |
|---|---|---|
| L + H⁺ ↔ HL | 8.84 (1) | 8.84 |
| HL + H⁺ ↔ H₂L | 15.92 (1) | 7.08 |
| H₂L + H⁺ ↔ H₃L | 19.71 (1) | 3.79 |
| H₃L + H⁺ ↔ H₄L | 22.54 (2) | 2.83 |
| H₄L + H⁺ ↔ H₅L | 24.92 (3) | 2.38 |
| H₅L + H⁺ ↔ H₆L | 27.06 (4) | 2.14 |
| H₆L + H⁺ ↔ H₇L | 28.07 (4) | 1.01 |
| H₇L + H⁺ ↔ H₈L | 27.35 (6) | −0.72^{b} |
| Σlog K_{HqL} | 27.35 (6) | 27.35 (6) |

^{a}K_{HqL} defined as [H_{q}L]/([H][H_{q-1}L].
^{b}not evaluated at constant I = 0.16M NaCl. Charges omitted for simplicity. Values in brackets are the standard deviations.

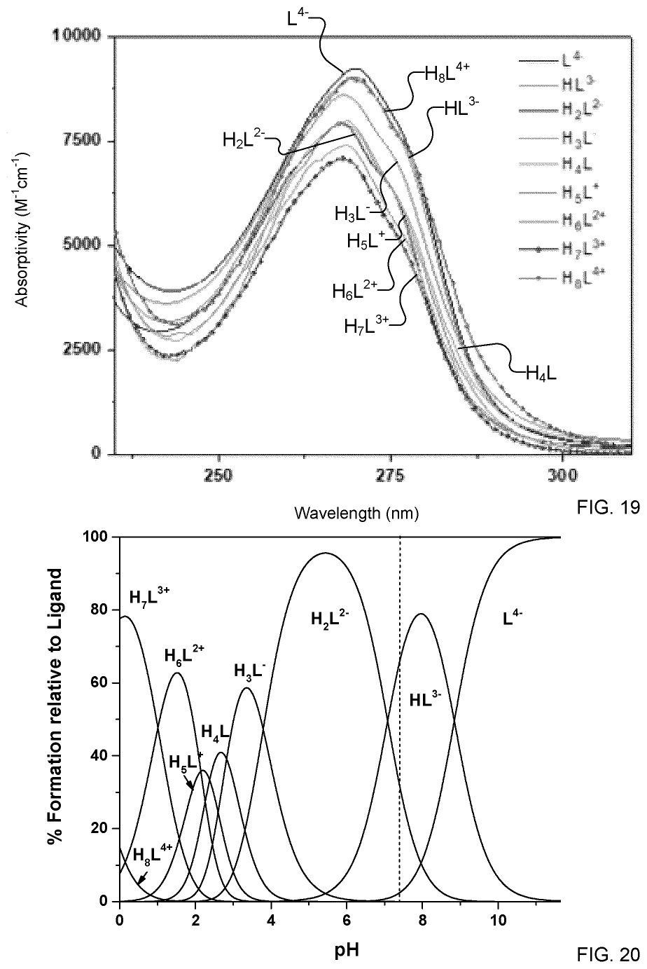

The protonation constants obtained for H₄noneunpaX follow the typical trend observed for similar polyaminocarboxylate-based ligands;^{6,12,13} wherein the first two dissociation events (species H₈L⁴⁺ and H₇L³⁺) can be attributed to the protonated pyridyl nitrogen donors. Deprotonation of each successive pyridine donor, with pKa −0.72 and 1.01 respectively, is concurrent with spectral changes in the absorbance spectrum of the ligand, where a large decrease in the absorptivity of the ligand is observed for the first dissociation event, FIGS. 19 and 20. Deprotonation of the two acetate carboxylic groups (log K₆=2.14 and log K₅=2.38), is followed by the two picolinate donors (log K₄=2.83 and log K₃=3.79), while the final two deprotonations are assigned to dipyridyl (log K₂=7.08) and iminodiacetate (log K₁=8.84) terminal amines respectively. The protonation constant measured for species HL is comparable to other ligands containing the iminodiacetate moiety (e.g. N-(2-hydroxyethyl)iminodiacetic acid (HEIDA), log K₁=8.68).²⁶ In contrast, the second terminal amine in H₄noneunpaX is less basic (log K₂=7.08) than the equivalent donor group in H₄noneunpa/Oxyaapa (log K₂=7.63)¹³ which can be attributed to the stronger electron-withdrawing effect induced by placing two picolinate donors on the same nitrogen atom, in addition to the steric constraints at this position, which may disfavour protonation. The larger difference in basicity between the terminal amines of H₄noneunpaX, in comparison to H₄noneunpa/Oxyaapa, may be advantageous for metal ion coordination, whereby the less basic dipyridyl nitrogen favours complexation at a lower pH, while the more basic iminodiacetate nitrogen will act as a stronger donor group to metal ions.

To evaluate the affinity of H₄noneunpaX towards metal ions of medicinal importance—e.g. Sc³⁺, In³⁺, Lu³⁺, Dy³⁺, Gd³⁺, Sm³⁺, La³⁺— the complex formation equilibria were studied using both combined potentiometric-spectrophotometric titrations, and in all cases, acidic in-batch UV-vis spectrophotometric titrations below pH 2. H₄noneunpaX showed a high affinity to all metal ions investigated, with metal complexation observed to begin below pH≈1. Table 4 presents the stability constants with each metal ion.

TABLE 4
Stability constants (log K_{ML}, log K_{MHL} and log K_{M(OH)L})^{a} of H₄noneunpaX with
M³⁺ = Sc³⁺, In³⁺, Lu³⁺, Dy³⁺, Gd³⁺, Sm³⁺, La³⁺ metal ions at 25° C. and I = 0.16M NaCl.

| | Sc³⁺ | In³⁺ | Lu³⁺ | Dy³⁺ | Gd³⁺ | Sm³⁺ | La³⁺ |
|---|---|---|---|---|---|---|---|
| log K_{ML} | 23.57(2) | 27.64(2) | 21.93(3) | 21.99(5) | 20.87(4) | 21.04(2) | 18.45(4) |
| log K_{MHL} | 3.07(1) | 6.17(3) | 2.28(3) | 2.29(2) | 2.80(1) | 2.77(1) | 3.22(2) |
| log K_{M(OH)L} | 10.89(2) | 8.58(4) | — | — | — | — | — |
| log K_{M(OH)2L} | — | 9.06(4) | — | — | — | — | — |
| pM^{b} | 22.9 | 26.5 | 21.3 | 21.3 | 20.2 | 20.4 | 17.8 |

^{a}K_{ML} defined as [ML]/([M][L]); K_{MHL} defined as [MHL]/([ML][H]); K_{M(OH)L} defined as [M(OH)L]/([ML][OH]) (charges omitted for clarity).
^{b}pM defined as −log[M_{free}] when [L] = 10 μM; [M] = 1 μM at pH 7.4.

H₄noneunpaX shows a high thermodynamic preference for complexation with trivalent Ln³⁺ ions, as indicated by the high stability constants (log K_{ML}) and pM values, Table 4. Notably, these values are directly comparable to those previously determined for H₄noneunpa/Oxyaapa,¹³ with H₄noneunpaX exhibiting marginally higher stability constants across the Ln³⁺ series, implying a small thermodynamic benefit to this inverted structural arrangement (which may be a result of a degree of preorganisation of the binding cavity of the ligand). Furthermore, the pM values obtained for H₄noneunpaX with Ln³⁺ ions exceed those of the current gold-standard chelators DOTA and DTPA by several log units (e.g. pLu=17.1 and 19.1 respectively).¹¹ While the stability constants and pM values are useful parameters for comparing the metal scavenging abilities of different chelating ligands, they do not necessarily correlate well with in vivo stability, another aspect for consideration is the speciation behaviour of a given metal complex as a function of pH.

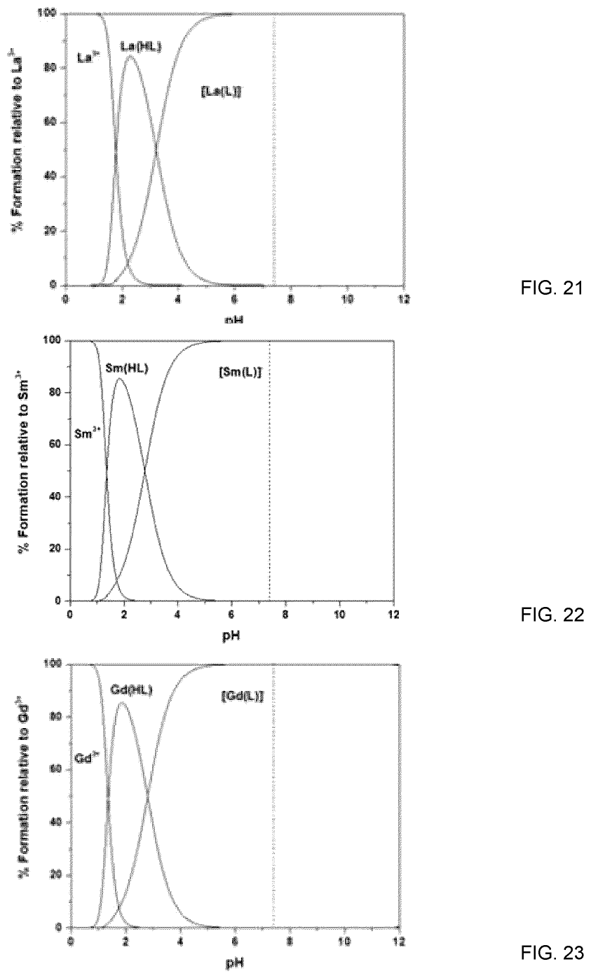

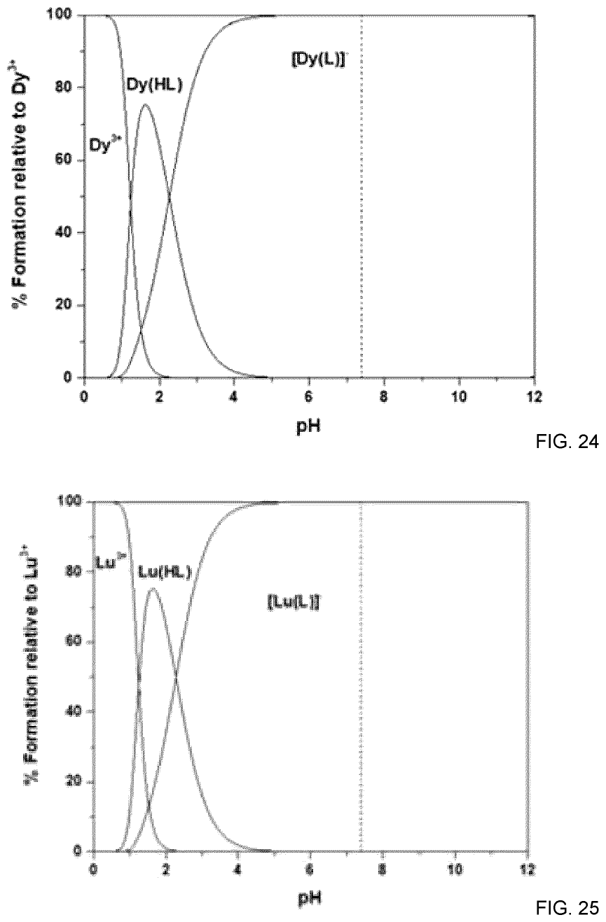

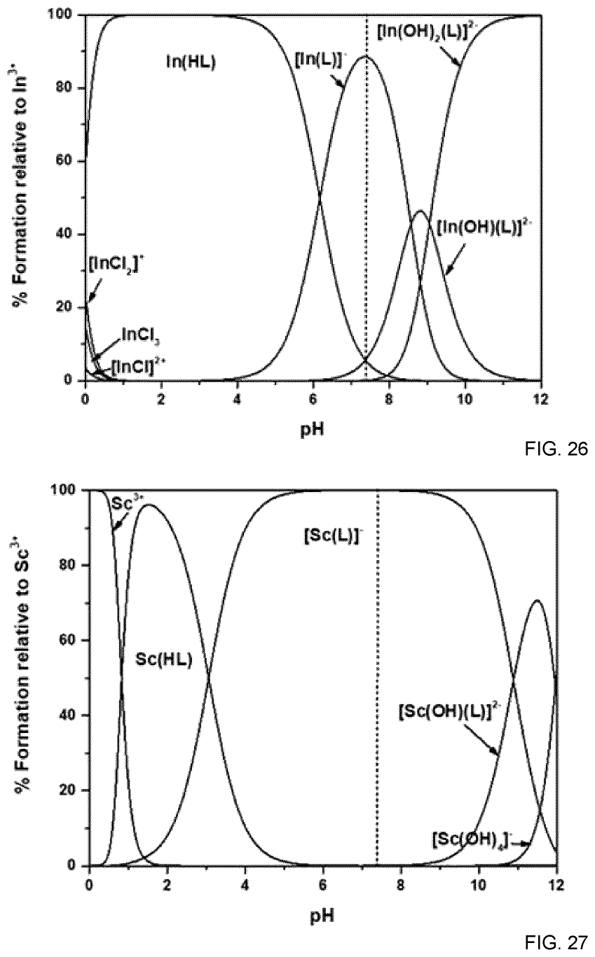

Speciation plots of the metal complexation of H₄noneunpaX with trivalent lanthanide ions show an interesting relationship, with the formation of the protonated MHL species at acidic pH, which is followed by a single transformation to the dominant [ML]⁻ species across a broad pH range, and the absence of any hydroxo species even under very basic conditions (pH=11.5), FIGS. 21-27. This is in contrary to many similar chelating ligands with Ln³⁺ ions previously reported—H₄octapa, H₄pypa, H₄octox—which all show the formation of one or more hydroxo species (e.g. [M(OH)L], [M(OH)₂L)]) under basic conditions (pH>9.0) and is typically attributed to deprotonation of a coordinated water molecule in the complex.^{7,9,12} Under physiological conditions (pH 7.4), a single species ([ML]⁻) is observed for all Ln³⁺ complexes of H₄noneunpaX, which is favourable for in vivo applications as different isomers can exhibit varying pharmacokinetic characteristics and biodistribution profiles.

Combined potentiometric-spectrophotometric titrations of H₄noneunpaX with smaller trivalent metal ions show a different relationship to the Ln³⁺ series. Higher stability constants were determined for Sc³⁺ and In³⁺, compared to Ln³⁺, as is typically observed owing to the higher charge density of these metal centres, Table 4. In the case of Sc³⁺, complex formation occurs below pH≈0.5 to give the neutral MHL species, which is deprotonated with increasing pH to give the anionic [ML]⁻ species. This transformation occurs in a similar range to the Ln³⁺ complexes of H₄noneunpaX (pKa=2.28-3.22) and may be attributed to the deprotonation of one of the carboxylate donors. In contrast, the In³⁺ complex shows different thermodynamic behaviour in solution. The MHL species deprotonates with a higher pKa to form the anionic [ML]⁻ species (pKa=6.17(3)) which is assigned to the tertiary amine of the iminodiacetate moiety, which remains protonated early on in the complexation. This is consistent with the NMR characterisation of the [In(noneunpaX)]⁻ complex which showed a single species at pH 4.0 in which the two acetate donor groups remain unbound to the metal centre. As in the case of the Sc³⁺ complex, the formation of hydroxo species is observed at higher pH, which without being bound by theory is accounted for by considering the mismatch between the size of these small metal ions and the ligand binding cavity.

All potentiometric titrations were carried out with a Metrohm Titrando 809 and a Metrohm Dosino 800 with a Ross combined electrode. Direct titrations were recorded using a Varian Cary 60 UV-vis spectrophotometer (200-350 nm spectral range) equipped with an optic dip probe (0.2 cm path length), while acidic in-batch experiments were measured with a standardised glass cuvette (1 cm path length). A temperature-controlled (298 K) 20 mL glass cell with an inlet-outlet adapter for nitrogen gas purging (purified via a 10% NaOH solution to exclude CO₂ prior to and during each titration) was used as a titration cell. The electrode was calibrated daily in hydrogen ion concentration by direct titration of HCl with freshly prepared NaOH solution, and the results were analysed with the Gran procedure⁵⁵ to determine the standard potential (E°) and the ionic product of water (pK_{w}), at 298 K, with 0.16 M NaCl as a supporting electrolyte. Solutions under study were titrated with carbonate-free NaOH solution (˜0.16 M) which was standardized against freshly recrystallized potassium hydrogen phthalate.

The protonation equilibria of H₄noneunpaX were evaluated through combined potentiometric-spectrophotometric titrations of solutions containing the ligand ([L]=9.57×10⁻⁴ M) in 0.16 M NaCl (T=298 K, I=0.2 cm). Electromotive force (EMF) values and UV-vis spectra were recorded after each addition of NaOH, and the apparatuses synchronized to have consistent time-intervals (30 s) between each addition/equilibration and data acquisition. Further acidic in-batch UV-vis spectrophotometry studies were performed to obtain the protonation constants for the most acidic ionisable protons of H₄noneunpaX, which were below the threshold of the pH electrode (pH<2). Separate solutions of the ligand were prepared at constant molarity ([L]=1×10⁻⁴ M) with differing amounts of HCl (0.1 M and 3.0 M), while maintaining a constant ionic strength (0.16 M NaCl) wherever possible. In each case, the solution stoichiometry was used to calculate the equilibrium concentration of H⁺ ions, following the Hammett acidity function (H₀) for determination of acidity in high concentration acid solutions. All eight protonation constants for H₄noneunpaX were determined through refinement of the experimental data using HypSpec2014²⁴ and Hyperquad2013²⁵ software.

The complex formation equilibria of H₄noneunpaX with Sc³⁺, In³⁺, Lu³⁺, Dy³⁺, Gd³⁺, Sm³⁺, and La³⁺ were evaluated using both combined potentiometric-spectrophotometric titrations, and acidic in-batch UV-vis spectrophotometric titrations below pH 2. For combined potentiometric-spectrophotometric titrations (spectral range: 200-350 nm), solutions containing a 1:1 molar ratio of metal-to-ligand ([M³⁺]≈[H₄noneunpaX]=8.40×10⁻⁴ M) were titrated against NaOH solution at T=298 K, I=0.2 cm. Acidic in-batch UV-vis spectrophotometric measurements (spectral range: 200-350 nm) were carried out using sets of solutions containing a 1:1 molar ratio of metal-to-ligand ([M³⁺]≈[H₄noneunpaX]≈1×10⁻⁴ M) with differing amounts of HCl (0.1 M and 3.0 M), to give samples ranging from pH 0-2. Measurements were recorded at T=298 K, I=1.0 cm, the Hammett acidity function (H₀) was used determination of the pH in highly acidic solutions rather than electrode potentials. Metal solutions were prepared from atomic absorption spectroscopy (AAS) standards, which were previously evaluated using Gran's method of titrating equimolar solutions of a given metal ion and Na₂H₂EDTA against NaOH to determine the acid concentration.

All the potentiometric measurements were processed using the Hyperquad2013²⁵ software, while spectroscopic measurements were analysed using HypSpec2014.²⁴ Proton dissociation constants corresponding to hydrolysis of the Sc³⁺, In³⁺, Lu³⁺, Dy³⁺ Gd³⁺, Sm³⁺, and La³⁺ aqueous ions used in the calculations were taken from Baes and Mesmer. Speciation diagrams were generated with the calculated protonation constants and stability constants using Hyss software.⁵⁶

Each species generated in the investigated systems can be defined in accordance with the general equilibrium equation pM+qH+rL=MₚH_{q}Lᵣ (charges omitted). Wherein, a complex containing a metal ion, M, proton, H and ligand, L, has the general formula MₚH_{q}Lᵣ. The stoichiometric index p may also be 0 in the case of protonation equilibria, and negative values of q refers to proton removal from coordinated water, equivalent to hydroxide ion addition during formation of the complex. The overall equilibrium constant for the formation of the complexes MₚH_{q}Lᵣ from its components is designated as log β. Stepwise equilibrium constants log K correspond to the difference in log units between the overall constants of sequentially protonated (or hydroxide) species. pM is defined as (−log[Mⁿ⁺]_{free}) and is calculated from the stability constants obtained for each studied system at [Mⁿ⁺]=1 μM, [L^{x-}]=10 μM, pH 7.4 and 25° C.⁵⁷

### Example 4.0—Radiolabelling Studies

Radiolabelling studies with [⁴⁴Sc]Sc³⁺, [¹¹¹ In]In³⁺, [¹⁷⁷Lu]Lu³⁺, [¹⁵⁵Tb]Tb³⁺, [²¹³Bi]Bi³⁺ and [²²⁵Ac]Ac³⁺ have been performed to investigate the variation in metal ion affinity with changes in ionic radii across a broad size range and coordination number. ²²⁵Ac (t_{1/2}=9.92 days) is of high interest for applications in targeted alpha therapy (TAT) owing its long half-life and the potency of particulate radiation emitted within its decay scheme. Suitable companion radionuclides for [²²⁵Ac]Ac³⁺ with comparable coordination characteristics and imageable decay properties are required to perform accurate staging of disease progression and assessment of patient suitability for treatments.

The relatively long-lived radionuclides [¹¹¹In]ln³⁺ (t_{1/2}=2.83 days)²⁷ and [¹⁵⁵Tb]Tb³⁺ (t_{1/2}=5.51 days)²⁰ are suitable for SPECT imaging owing to the emission of high abundance, low energy γ-rays (171 and 245 keV [¹¹¹In]²⁷; 44, 87 and 105 keV [¹⁵⁵Tb]²⁰) in their decay schemes, and are well-matched to the long half-life of [²²⁵Ac]Ac³⁺. [⁴⁴Sc]Sc³⁺ is a promising candidate for PET imaging, owing to its long physical half-life (t_{1/2}=3.97 h) and high positron branching ratio (E_{β}_{⁺}=632 keV, 94.3%), in addition, the companion radioisotope [⁴⁷Sc]Sc³ (t_{1/2}=3.35 days) may find utility for β⁻-therapy (E_{β}_{^{−}}=162 keV, 100%).²⁸

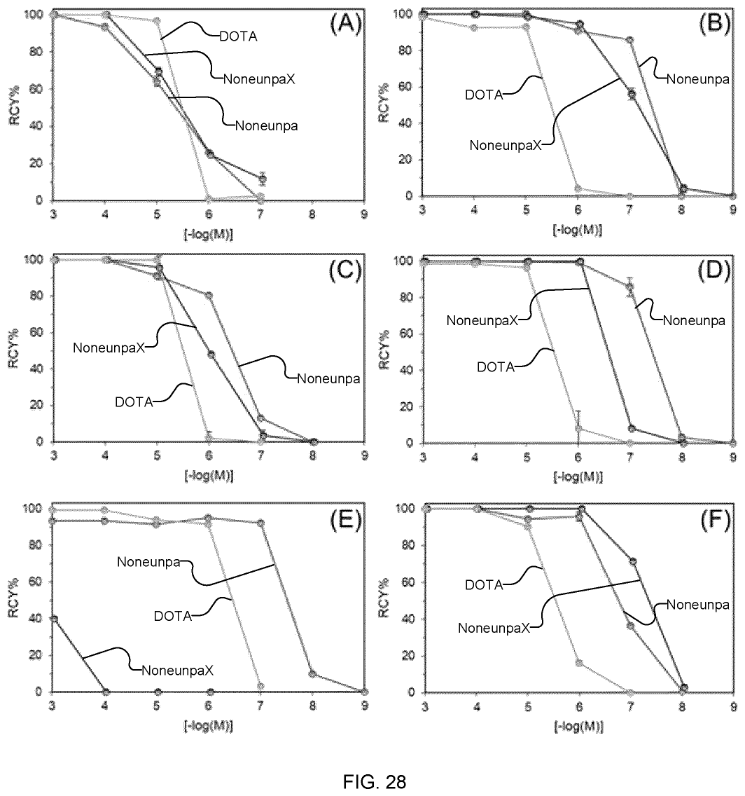

Concentration-dependent radiolabelling studies have shown H₄noneunpaX to be a highly versatile chelator, exhibiting high affinity for all metal ions tested, with the exception of [²¹³Bi]Bi³⁺, FIG. 28. All reactions with H₄noneunpa and H₄noneunpaX were carried out at RT and monitored over 10 minutes, with the exception [²¹³Bi]Bi³⁺ which were monitored after 5 minutes, within 10 minutes post-generator elution (n=4−8). Reactions with DOTA were carried out at elevated temperatures and monitored over 30-60 minutes.

Significantly, quantitative radiochemical conversion (RCC) was achieved within 10 minutes at room temperature, a notable advantage over the widely applied ‘gold-standard’ chelator DOTA. Optimal radiochemical yields were obtained under exceptionally mild conditions (pH 7.0, RT, 10 min.) which are compatible with thermally sensitive biological targeting vectors (monoclonal antibodies). H₄noneunpaX showed comparable coordination characteristics to H₄noneunpa; each chelate was successfully radiolabelled at high molar activities with [¹¹¹In]In³⁺ (54 GBq/μmol), [¹⁵⁵Tb]Tb³⁺ (1.0 GBq/μmol), [¹⁷⁷Lu]Lu³⁺ (2.0 GBq/μmol) and [²²⁵Ac]Ac³⁺ (134 MBq/μmol).

Concentration-dependent radiolabelling studies of H₄noneunpa and H₄noneunpaX with [⁴⁴Sc]Sc³⁺ showed poor radiometal ion compatibility; whereby low RCYs were achieved at high ligand concentration. These results were anticipated based on the small ionic radius (0.870 Å, CN=8) and chemical hardness of the Sc³⁺ ion, which prefers coordination numbers of 6-8.¹⁸ However, these results further suggest no significant difference in metal ion affinity between symmetric and asymmetric ligands based on this framework.

The distinct contrast in radiolabelling efficiency exhibited for [²¹³Bi]Bi³⁺ by each chelate was somewhat surprising given the similarities in donor atoms and the observed trends for other trivalent metal ions. However, without being bound by theory, this deviation can be rationalised by consideration of the coordination characteristics of the Bi³⁺ ion. Bi³⁺ possesses a similar ionic radius to Ac³⁺ (1.170 Å (CN=8) versus 1.220 Å (CN=9), for Bi³⁺ and Ac³⁺ respectively),^{18,19} but is of intermediate chemical hardness, exhibiting a stronger preference for intermediate hardness donor groups (e.g. nitrogen, pyridine) over hard ionic donors (oxygen, phenolates). Bi³⁺ is also known to exhibit a stereochemically active 6 s² lone pair in some of its coordination complexes, which can have significant impacts on the favoured conformational geometries of the chelating ligand and their effective denticities.^{29,30}

### Example 5.0—Human Serum Stability Studies

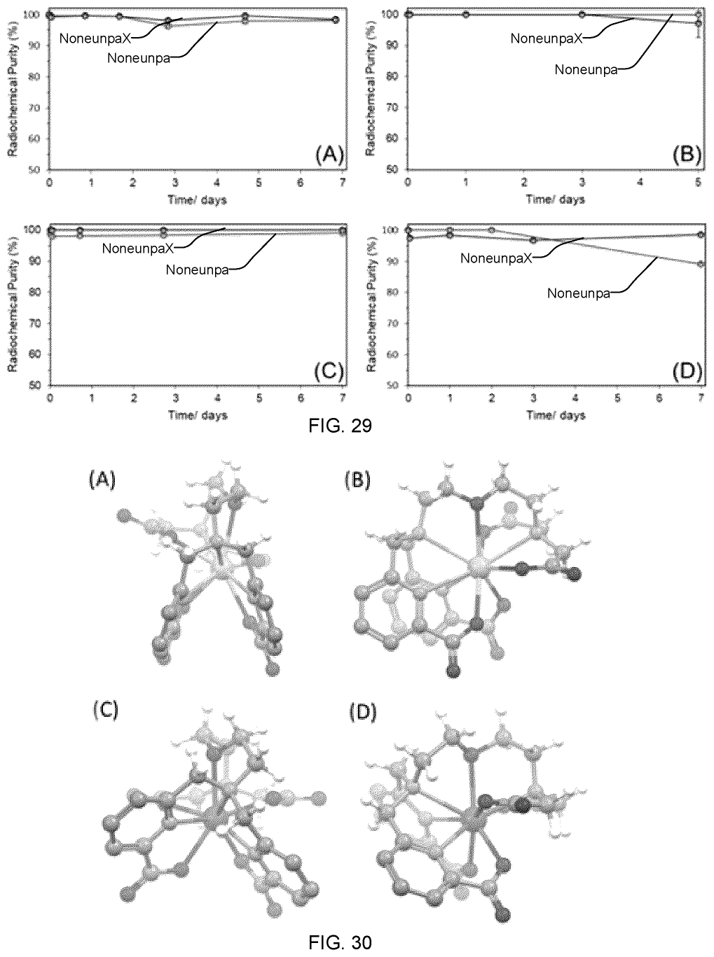

A series of serum stability challenge assays were undertaken in order to determine the kinetic inertness of resulting complexes of H₄noneunpaX and H₄noneunpa with [¹¹¹In]In³⁺, [¹⁵⁵Tb]Tb³⁺, [¹⁷⁷Lu]Lu³⁺ and [²²⁵Ac]Ac³⁺ in the presence of competing endogenous metal binding proteins. Results are shown in FIG. 29. In the case of H₄noneunpaX, incubation of the radiolabelled complexes at 37° C. in human serum showed no release of bound radiometal over 5-7 days, indicating high kinetic inertness and potential for in vivo application. The radiolabelled complexes of H₄noneunpa exhibited similar kinetic inertness to H₄noneunpaX, maintaining >97% radiochemical purity over 5-7 days, with the exception of [²²⁵Ac][Ac(noneunpa)]⁻ which showed an initial drop in radiochemical integrity of ˜10% over the course of the study. All experiments were performed at 37° C. and monitored via radio-iTLC (n=3).

### Example 6.0—Computational Studies

To gain insights into the solution structures of the metal complexes of H₄noneunpaX, density functional theory (DFT) calculations were carried out using Gaussian 16 (revision B.01) in the polarizability continuum model (PCM).^{31,32} Geometrical optimisations were performed with the hybrid Perdew-Burke-Ernzerhof (PBE(0) exchange-correlation functional, using the relatively small core quasi-relativistic effective core potentials (ECP28/60MWB) and associated valence basis-sets for metal ions (La³⁺, Lu³⁺, Bi³⁺), while light atoms (C, H, N, O) were modelled up to Def2TZVP levels of theory.³³⁻³⁵ Optimised structures for all complexes, with cartesian coordinates and calculated thermodynamic energy values were determined. Initial geometries were generated using Avogadro (version 1.2.0) to provide input coordinates for calculations.⁵¹ Solvation effects were modelled using the integrated equation formalism polarizability continuum model (IEF-PCM) for all metal complexes.³² Vibrational frequency analysis was carried out on the final optimised geometries to confirm the obtained structures were true energy minima of the potential energy surface. Natural bond orbital (NBO) analysis was performed using NBO (version 3.1) within Gaussian 16.³⁶ Contour plots of the electron-density encompassing the 6 s² lone pair in [Bi(noneunpaX)]⁻ were generated using Multiwfn software.⁵²

The distribution of donor arm substituents within the framework of H₄noneunpaX gives rise to four possible conformational isomers on metal ion coordination (with a coordination number of 9) which arise from the relative orientation of the 5-membered chelate rings formed between the ethylene bridges of the ligand and the metal centre (λ vs. δ). The solution structures of H₄noneunpa/Oxyaapa with La³⁺, Lu³⁺ and In³⁺ have been thoroughly investigated in previous studies.^{8,13}

Geometric optimisations of the La³⁺ and Lu³⁺ complexes of H₄noneunpaX generated structures with fully saturated metal coordination spheres, whereby all nine donor atoms from the ligand were bound to the metal centres. In the case of [La(noneunpaX)]⁻, geometric optimisation favoured a single conformational isomer with the symmetric δδ configuration being the lowest in energy, while the [Lu(noneunpaX)]⁻ complex favoured a different asymmetric isomer with a twisted λδ conformation, FIG. 30. Intriguingly, this is in contrast to the complexation reported for H₄noneunpa, which was found to form an asymmetric arrangement in the La³⁺ complex and a fully symmetric conformation in the Lu³⁺ complex.

The structure of [La(noneunpaX)]⁻ shows a high degree of symmetry, wherein the two picolinic acid donors adopt the same relative arrangement, with both pyridine rings eclipsed (parallel), while the two acetate donors also adopt the same relative arrangement in an antiparallel orientation relative the central plane of symmetry. This characterisation is consistent with the experimental NMR results of the La³⁺ complex which indicated the formation of a single, symmetric isomer in solution. Additionally, from the DFT calculated structure, the methylene protons adjacent to the acetate groups (Hₐ/H_{b} and H_{a′}/H_{b′},) are shown in near identical chemical environments, thus giving rise to two pairs of diastereotopic doublets in the ¹H NMR spectrum with similar chemical shifts (δ_{ab}=3.37 ppm, δ_{a′b′}=3.31 ppm). By contrast, the solution phase structure of [Lu(noneunpaX)]⁻ shows a single asymmetric isomer, wherein a change in backbone geometry to the λδ conformation causes a shift in the relative arrangement of the picolinic acid donors to an orthogonal configuration. This conformational shift is also further supported by the ¹H NMR characterisation of the Lu³⁺ complex, which exhibits sharply resolved, diastereotopic doublets for the four methylenic protons neighbouring the picolinate donor arms. The large differences in chemical shift for mutually coupled protons H_{g}/H_{g′}, (Δδ=0.78 ppm) and Hₖ/H_{k′} (Δδ=0.74 ppm) can be clearly accounted for by the distinct local environments adopted in the rigid [Lu(noneunpaX)]⁻ complex, FIG. 30, panel (C).

### Example 7.0—Synthesis and Characterization of Bifunctional H₄noneunpaX-Bn-NCS

The synthetic approached developed towards bifunctional H₄noneunpaX-Bn-NCS having the structure (13)

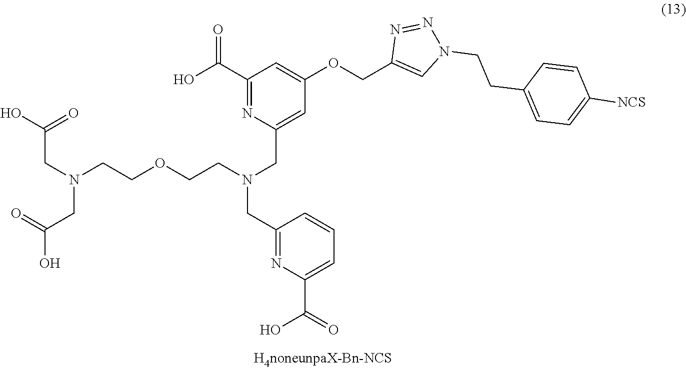

involved a straightforward, stepwise synthesis as outlined in Scheme 2, in which an emphasis on single modifications and functional group interconversions were selected in each step, in order to simplify purification of synthetic intermediates, minimise potential by-product formation, and allow reaction scalability.

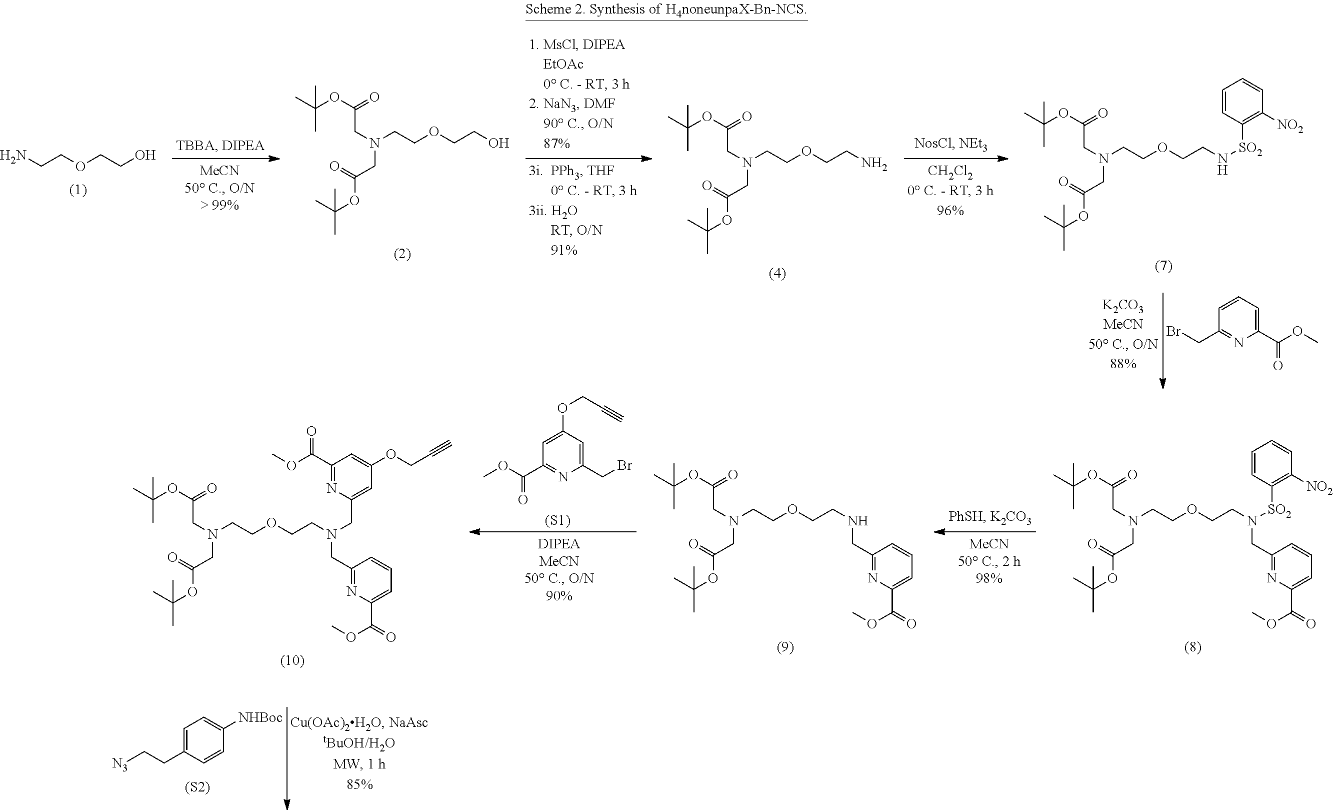

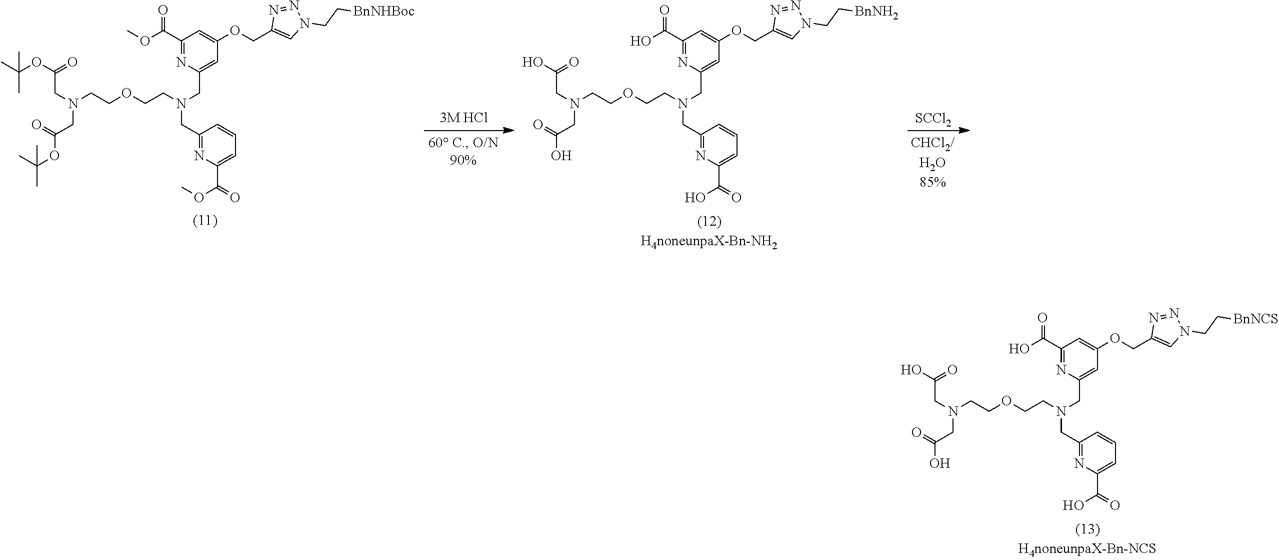

The synthesis of bifunctional H₄noneunpaX-Bn-NCS (13) proceeds through a pathway analogous to H₄noneunpaX, utilising the same synthetic intermediates (compounds (1)-(4)) to provide primary amine (4) which was protected using 2-nitrobenzenesulphonyl chloride to give the corresponding Nosyl-protected sulfonamide (7) in high yield. N-alkylation of sulfonamide (7) with methyl (6-bromomethyl)picolinate under basic conditions was achieved through mild heating of the reaction mixture overnight, to generate the singly alkylated product, cleanly in high yields. Removal of the Nosyl-protecting group was performed upon treatment of compound (8) with thiophenol to give the corresponding secondary amine (9). An excess of thiophenol (3 equiv.) was found to be necessary to achieve complete deprotection of compound (8) and thus avoid tedious purification of the desired product. Subsequent N-alkylation of secondary amine (9) using a structurally modified derivative of the protected picolinic acid electrophile (S1) was achieved through mild heating overnight to give the protected bifunctional ligand (10) with an alkyne appendage, suitable for click-based bioconjugation. The alkyne-derivatized picolinate electrophile (S1) was prepared separately starting from chelidamic acid, as shown in Scheme 3. Alkyne (10) was subsequently reacted with the bifunctional azide linker (S2) using Husigen's 1,3-dipolar cycloaddition (Click chemistry) using CuSO₄·5H₂O as an in-situ source of Cu(I) catalyst and sodium ascorbate as a reductant. Syntheses for the bifunctional azide linker (S2) are also outlined in Scheme 4. Compound (11) was firstly treated with an excess of sodium sulfide to precipitate any traces of Cu(I/II) chelated by the protected ligand prior to deprotection of the methyl, tert-butyl and Boc protected groups using 3 M HCl. Purification of H₄noneunpaX-Bn-NH₂ (12) was achieved via RP-HPLC and the product isolated as the corresponding HCl salt by co-evaporation with 3 M HCl. Conversion of the aniline (12) to the isothiocyanate (13) was carried out using standardised literature approaches involving thiophosgene,³⁷ and the product isolated via RP-HPLC.

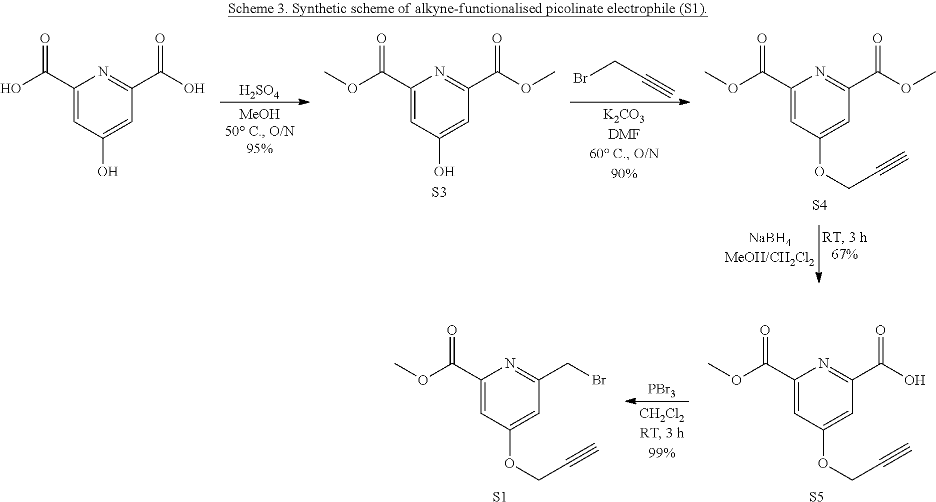

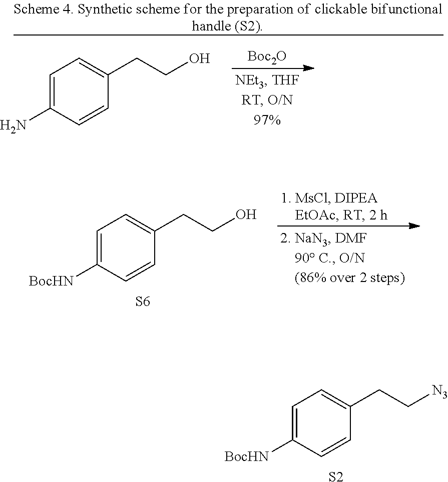

Di-tert-butyl 2,2′-((2-(2-((2-nitrophenyl)sulfonamido)ethoxy)ethyl)azanediyl)diacetate (7). Di-tert-butyl 2,2′-((2-(2-aminoethoxy)ethyl)azanediyl)diacetate (4) (2.35 g, 7.16 mmol) was dissolved in dry CH₂Cl₂ (40 mL) and cooled to 0° C. Triethylamine (2.00 mL, 1.45 g, 14.3 mmol, 2 equiv.) was added to the reaction mixture, followed by slow addition of 2-nitrobenzenesulphonyl chloride (1.59 g, 7.16 mmol). The resulting pale-yellow solution was stirred at 0° C. for 1 h, then allowed to warm to RT and stirred for a further 5 h. Upon completion, the reaction mixture was diluted with CH₂Cl₂ (40 mL) and extracted with de-ionised H₂O (2×50 mL) and brine (50 mL). The organic phase was evaporated in vacuo and the resulting residue purified via silica gel chromatography (Combiflash automated purification system; A: hexanes, B: EtOAc, 100% A to 40% B). The title compound was attained as a pale-yellow oil (3.51 g, 96%). ¹H NMR (400 MHz, CDCl₃, 298 K) 8.14-8.12 (1H, m, 11-CH), 7.85-7.82 (1H, m, 9-CH), 7.75-7.70 (2H, m, 8- and 11-CH), 6.13 (1H, t, ³J=5.6 Hz, 7-NH), 3.54-3.49 (4H, m, 4- and 5-CH₂), 3.45 (4H, s, 2-CH₂), 3.28 (2H, q, ³J=5.5 Hz, 6-CH₂), 2.87 (2H, t, ³J=5.6 Hz, 3-CH₂), 1.45 (18H, s, 1-C(CH₃)₃). ¹³C{¹H} NMR (100 MHz, CDCl₃,298 K) 170.7 (3-C), 148.1 (14-C), 134.0 (11-C), 133.4 (9-C), 132.6 (12-C), 130.9 (13-C), 125.2 (10-C), 81.1 (2-C), 70.1 (6-C), 68.9 (7-C), 56.7 (4-C), 53.2 (5-C), 43.7 (8-C), 28.2 (1-C). ESI-MS (MeOH) 518.7 [M+H]⁺. R_{f}=0.50 (Hexanes/EtOAc; 2:1).

Di-tert-butyl-2,2′-((2-(2-((N-((6-(methoxycarbonyl)pyridin-2-yl)methyl)-2-nitrophenyl) sulfonamido)ethoxy)ethyl)azanediyl)diacetate (8): Methyl (6-bromomethyl) picolinate (884 mg, 3.84 mmol) was added to a solution of di-tert-butyl 2,2′-((2-(2-((2-nitrophenyl)sulfonamido) ethoxy)ethyl)azanediyl)diacetate (7) (1.98 g, 3.84 mmol) in dry MeCN (60 mL). The solution was stirred for 15 minutes at RT after which K₂CO₃ (1.06 g, 7.68 mmol, 2.0 equiv.) was added and the resulting suspension heated at 50° C. overnight. Upon completion, the inorganic salts were separated via centrifuge, washed with CH₂Cl₂ (3×10 mL) and the combined organic phase was evaporated in vacuo. The resulting residue was re-dissolved in CH₂Cl₂ (75 mL), washed with de-ionised H₂O (3×75 mL) and brine (75 mL) and dried over Na₂SO₄. The organic phase was evaporated in vacuo and the resulting residue purified via silica gel chromatography (Combiflash automated purification system; A: CH₂Cl₂, B: MeOH; 100% A to 5% B). The title product was attained as a pale-yellow oil (2.26 g, 88%). ¹H NMR (400 MHz, CDCl₃, 298 K) 8.13-8.1 (1H, m, 11-CH), 7.98 (1H, d, ³J=7.1 Hz, 14-CH), 7.81 (1H, t, ³J=7.1 Hz, 13-CH), 7.68 (1H, d, ³J=7.1 Hz, 12-CH), 7.66-7.62 (3H, m, 8-, 9- and 10-CH), 4.83 (2H, s, 7-CH₂), 3.95 (3H, s, 15-CH₃), 3.54 (2H, t, ³J=5.3 Hz, 6-CH₂), 3.47 (2H, t, ³J=5.3 Hz, 5-CH₂), 3.37 (4H, s, 2-CH₂), 3.35 (2H, t, ³J=6.0 Hz, 4-CH₂), 2.71 (2H, ³J=6.0 Hz, 3-CH₂), 1.41 (18H, s, 1-C(CH₃)₃). ¹³C{¹H} NMR (100 MHz, CDCl₃, 298K) 170.6 (3-C), 165.4 (21-C), 157.4 (16-C), 148.1 (20-C), 147.4 (14-C), 137.9 (18-C), 133.5 (11-C), 133.4 (9-C), 131.8 (12-C), 131.0 (13-C), 125.4 (17-C), 124.3 (10-C), 124.0 (19-C), 80.9 (2-C), 70.0 (6-C), 68.6 (7-C), 56.5 (4-C), 53.9 (15-C), 53.3 (5-C), 52.8 (22-C), 48.1 (8-C), 28.1 (1-C). ESI-MS (MeOH) 667.3 [M+H]⁺. R_{f}=0.30 (CH₂Cl₂/MeOH; 99:1).

Di-tert-butyl 2,2′-((2-(2-(((6-(methoxycarbonyl)pyridin-2-yl)methyl)amino) ethoxy)ethyl) azanediyl)diacetate (9). Thiophenol (1.05 mL, 10.2 mmol, 3.0 equiv.) was added to a suspension of di-tert-butyl 2,2′-((2-(2-((N-((6-(methoxycarbonyl)pyridin-2-yl)methyl)-2-nitrophenyl)sulfonamido)ethoxy)ethyl)azanediyl)diacetate (8) (2.26 g, 3.39 mmol) and K₂CO₃ (937 mg, 6.78 mmol, 2.0 equiv.) in dry MeCN (60 mL). The reaction mixture was heated to 50° C. and stirred for 2 h. Upon completion, the inorganic salts were separated via centrifuge, washed with CH₂Cl₂ (3×10 mL) and the combined organic phase evaporated in vacuo. The resulting residue was re-dissolved in CH₂Cl₂ (50 mL) and washed with de-ionised H₂O (2×50 mL) and brine (50 mL). The organic phase was evaporated in vacuo and the resulting oil purified via silica gel chromatography (Combiflash automated purification system; A: CH₂Cl₂, B: MeOH; 100% A to 10% B) to give the title product as a pale-yellow oil (1.61 g, 98%). ¹H NMR (400 MHz, CDCl₃, 298 K) 8.00 (1H, d, ³J=7.4 Hz, 11-CH), 7.81 (1H, t, ³J=7.4 Hz, 10-CH), 7.66 (1H, d, ³J=7.4 Hz, 9-CH), 4.05 (2H, s, 8-CH₂), 3.99 (3H, s, 12-CH₃), 3.61-3.56 (4H, m, 4- and 5-CH₂), 3.49 (4H, s, 2-CH₂), 2.94 (2H, t, ³J=5.8 Hz, 6-CH₂), 2.83 (2H, t, ³J=5.2 Hz, 3-CH₂), 2.26 (1H, br s, 7-NH), 1.44 (18H, s, 1-C(CH₃)₃). ¹³C {¹H} NMR (100 MHz, CDCl₃, 298 K) 170.8 (3-C), 165.8 (15-C), 160.5 (10-C), 147.4 (14-C), 137.5 (12-C), 125.6 (11-C), 123.5 (13-C), 80.9 (2-C), 70.1 (6-C), 70.0 (7-C), 56.6 (4-C), 54.9 (9-C), 53.4 (5-C), 52.8 (16-C), 48.8 (8-C), 28.1 (1-C). ESI-MS (MeOH) 482.2 [M+H]⁺. R_{f}=0.20 (CH₂Cl₂/MeOH; 9:1).

(^{t}Bu)₂(Me)₂noneunpaX(OCH₂CCH) (10). Methyl 6-(bromomethyl)-4-(prop-2-yn-1-yloxy)picolinate (122 mg, 0.415 mmol) was added to a suspension of di-tert-butyl 2,2′-((2-(2-(((6-(methoxycarbonyl)pyridin-2-yl)methyl)amino)ethoxy)ethyl)azanediyl)diacetate (200 mg, 0.415 mmol) and K₂CO₃(115 mg, 0.830 mmol) in dry MeCN (10 mL). The reaction mixture was heated to 50° C. and stirred overnight. Upon completion, the reaction mixture was cooled to room temperature, and the inorganic salts removed via centrifuge. The separated salts were washed with CH₂Cl₂ (3×10 mL) and the combined organic phase evaporated in vacuo. The resulting residue was re-dissolved in CH₂Cl₂ (25 mL) and washed with de-ionised H₂O (2×25 mL) and brine (25 mL). The volatiles were removed in vacuo and the crude product purified via silica gel chromatography (Combiflash automated purification system; A: CH₂Cl₂, B: MeOH; 100% A to 5% B). The title product was attained as a pale-yellow oil (249 mg, 88%). ¹H NMR (400 MHz, CDCl₃, 298 K) 7.98 (1H, dd, ³J=7.5 Hz, ⁴J=0.9 Hz, 10-CH), 7.88 (1H, d, ³J=7.5 Hz, 8-CH), 7.78 (1H, t, ³J=7.5 Hz, 9-CH), 7.59 (1H, d, ⁴J=2.5 Hz, 14-CH), 7.52 (1H, br d, ⁴J=2.5 Hz, 13-CH), 4.81 (2H, d, ⁴J=2.4 Hz, 16-CH₂) 4.01 (2H, br s, 7-CH₂), 3.98 (3H, s, 11-CH₃), 3.97 (5H, br m, 12-CH₂ and 15-CH₃), 3.57 (2H, t, ³J=5.5 Hz, 4-CH₂), 3.52 (2H, t, ³J=5.9 Hz, 5-CH₂), 3.45 (4H, s, 2-CH₂), 2.91 (2H, t, ³J=5.9 Hz, 6-CH₂), 2.81 (2H, t, ³J=5.5 Hz, 3-CH₂), 2.60 (1H, t, ⁴J=2.4 Hz, 17-CH), 1.42 (18H, s, 1-C(CH₃)₃). ¹³C{¹H} NMR (100 MHz, CDCl₃, 298 K) 170.7 (3-C), 165.8 (15-C), 165.7 (23-C), 165.1 (20-C), 162.6 (10-C), 160.6 (18-C), 148.9 (22-C) 147.3 (14-C), 137.4 (12-C), 126.1 (11-C), 123.6 (13-C), 111.8 (19-C), 111.0 (21-C), 80.8 (2-C), 77.1 (26-C), 76.9 (27-C), 70.4 (6-C), 69.3 (7-C), 60.8 (9-C), 60.6 (17-C), 56.6 (4-C), 55.9 (25-C), 54.0 (8-C), 53.5 (5-C), 53.0 (16-C), 52.9 (24-C), 28.1 (1-C). ESI-MS (MeOH) 685.3 [M+H]⁺.

(^{t}Bu)₂(Me)₂noneunpaX(Bn-NHBoc) (11). A solution of Cu(OAc)₂·H₂O (66 mg, 0.329 mmol, 1.0 equiv.) in de-ionised H₂O (1 mL) was added to a mixture containing (^{t}Bu)₂(Me)₂noneunpaX(OCH₂CCH) (225 mg, 0.329 mmol) and tert-butyl (4-(2-azidoethyl)phenyl)-carbamate (86 mg, 0.329 mmol) in ^{t}BuOH (1 mL). Sodium ascorbate (65 mg, 0.329 mmol, 1.0 equiv.) was added to the reaction mixture, which darkened progressively over 10 min to give a red-brown solution. The solution was warmed to 40° C. and stirred for 48 h. Upon completion, the dark brown mixture was treated with a solution Na₂S·9H₂O (790 mg, 3.29 mmol, 10 equiv.) dissolved in de-ionised H₂O (4 mL). Black CuS precipitated immediately on addition, and the suspension was stirred for a further 1 h at RT. The precipitate was separated by centrifuge, and the supernatant collected and evaporated under reduced pressure to remove excess ^{t}BuOH. The resulting aqueous phase was diluted with de-ionised H₂O (10 mL) and extracted with CH₂Cl₂ (3×15 mL). The combined organic phase was washed with de-ionised H₂O (10 mL) and brine (10 mL). The volatiles were removed in vacuo and the crude material purified via silica gel chromatography (Combiflash automated purification system; A: CH₂Cl₂, B: MeOH; 100% A to 10% B) to attain the title compound as an white aerated solid (211 mg, 76%). ¹H NMR (400 MHz, CDCl₃,298 K) 8.00 (1H, d, ³J=7.5 Hz, 10-CH), 7.90 (1H, d, ³J=7.5 Hz, 18-CH), 7.86 (1H, t, ³J=7.5 Hz, 9-CH), 7.60 (1H, d, ⁴J=2.2 Hz, 14-CH), 7.53 (1H, d, ⁴J=2.2 Hz, 13-CH), 7.45 (1H, s, 17-CH), 7.27 (2H, d, ³J=8.2 Hz, 21-CH), 6.98 (2H, d, ³J=8.2 Hz, 20-CH), 6.66 (1H, br s, 22-NH), 5.28 (2H, s, 25-CH₂), 4.59 (2H, t, ³J=7.1 Hz, 18-CH₂), 4.02 (2H, s, 12-CH₂), 3.99 (3H, s, 11-CH₃), 3.98 (3H, s, 15-CH₃), 3.96 (2H, s, 7-CH₂), 3.58 (2H, t, ³J=5.6 Hz, 4-CH₂), 3.53 (2H, t, ³J=5.8 Hz, 5-CH₂), 3.46 (4H, s, 4-CH₂), 3.17 (2H, t, ³J=7.1 Hz, 19-CH₂), 2.91 (2H, t, ³J=5.8 Hz, 6-CH₂), 2.81 (2H, t, ³J=5.6 Hz, 3-CH₂), 1.52 (9H, s, 23-C(CH₃)₃), 1.43 (18H, s, 1-C(CH₃)₃). ESI-MS (MeOH) 947.5 [M+H]⁺.

H₄noneunpaX-Bn-NH₂ (12). (^{t}Bu)₂(Me)₂noneunpaX(Bn-NHBoc) (200 mg, 0.211 mmol) was dissolved in 4 M HCl (3 mL) and heated at 60° C. overnight. Upon completion, the volatiles were evaporated in vacuo and the resulting residue purified via RP-HPLC (A: H₂O (0.1% TFA), B: MeCN; 95% A to 30% B over 40 min, Rₜ=20.3-22.3 min). Purified H₄noneunpaX-triazole-Bn-NH₂ was co-evaporated with 3 M HCl (3×2 mL) to give the corresponding HCl as a white solid (120 mg, 81%). ¹H NMR (400 MHz, D₂O, 298 K) 8.03 (1H, s, 14-CH), 7.94-7.83 (2H, m, 8- and 9-CH), 7.62 (1H, dd, ³J=6.3 Hz, ⁴J=2.0 Hz, 7-CH), 7.58 (1H, d, ⁴J=2.7 Hz, 12-CH), 7.24 (1H, d, ⁴J=2.7 Hz, 11-CH), 7.22 (2H, d, ³J=7.4 Hz, 17-CH), 7.15 (2H, d, ³J=7.4 Hz, 18-CH), 5.29 (2H, s, 13-CH₂), 4.74-4.65 (6H, m, 6-, 10- and 15-CH₂), 4.22 (4H, s, 1-CH₂), 3.97 (2H, t, 4-CH₂), 3.89 (2H, t, 3-CH₂), 3.76 (2H, t, 5-CH₂), 3.66 (2H, t, 2-CH₂), 3.23 (2H, t, 16-CH₂). ¹³C{¹H} NMR (100 MHz, D₂O, 298 K) 168.1 (17-C), 168.0 (1-C), 166.4 (13-C), 164.7 (20-C), 150.4 (8-C), 150.1 (15-C), 147.3 (12-C), 146.0 (19-C), 140.9 (22-C), 140.7 (10-C), 138.5 (29-C), 130.4 (27-C), 128.5 (26-C), 128.4 (9-C), 126.2 (23-C), 125.5 (11-C), 123.3 (28-C), 115.8 (16-C), 112.8 (18-C), 65.4 (5-C), 64.4 (4-C), 62.1 (21-C), 58.3 (7-C), 57.7 (14-C), 56.4 (3-C), 56.2 (6-C), 55.6 (2-C), 51.9 (24-C), 35.2 (25-C). LR-ESI-MS (H₂O) 707.6 [M+H]⁺

H₄noneunpaX-Bn-NCS (13). Thiophosgene (120 μL, 1.56 mmol) in CHCl₃ (660 μL) was added to a solution of H₄noneunpaX-Bn-NH₂-4HCl (88 mg, 0.103 mmol) in de-ionised H₂O (1 mL). The biphasic mixture was stirred vigorously in the dark overnight at RT, after which the two immiscible layers were allowed to separate. The aqueous phase was collected, washed with CHCl₃ (2×1 mL), and then lyophilised to attain the crude isothiocyanate product. The off-white residue was purified via semi-preparation RP-HPLC (A: H₂O (0.1% TFA), B: MeCN (0.1% TFA)): 0-6 min. (95% A to 24% B); 6-30 min. (24% B to 39% B); 30-35 min. (39% B to 100% B); Rₜ=25.4 min. Appropriate fractions were pooled and lyophilised to give the purified product as an off-white aerated solid (67 mg, 87%). ESI-MS (H₂O/MeCN) 749.3 [M+H]⁺, 747.3 [M−H]⁻.

Dimethyl-4-hydroxypyridine-2,6-dicarboxylate (S3) Chelidamic acid monohydrate (3.00 g, 14.9 mmol) was suspended in dry MeOH (50 mL) and H₂SO₄ (0.5 mL) added. The reaction mixture was heated at 50° C. overnight, then cooled to RT and the volatiles removed in vacuo. The crude residue was dissolved in sat. NaHCO₃ sol. (150 mL) and extracted with EtOAc (3×150 mL). The combined organic phase was evaporated in vacuo to attain the title compound as a white solid (3.02 g, 95%). ¹H NMR (300 MHz, CDCl₃, 298 K) 7.49 (2H, s, 2-CH), 3.97 (6H, s, 1-CH₃). ¹³C {¹H} NMR (75 MHz, CDCl₃,298 K) 187.5 (5-C), 163.5 (2-C), 143.1 (3-C), 118.6 (4-C), 53.7 (1-C).

Dimethyl 4-(prop-2-yn-1-yloxy)pyridine-2,6-dicarboxylate (S4) Propargyl bromide (80% wt.)(1.75 mL, 15.7 mmol, 1.1 equiv.) was added to a suspension of dimethyl-4-hydroxypyridine-2,6-dicarboxylate (3.02 g, 14.3 mmol) and K₂CO₃ (3.95 g, 28.6 mmol, 2.0 equiv.) in dry DMF (50 mL). The reaction mixture was heated at 50° C. overnight, then cooled to RT and the salts removed via vacuum filtration. The filtrate was evaporated in vacuo and the resulting residue purified via silica gel chromatography (Combiflash automated purification system; A: CH₂Cl₂, B: MeOH; 100% A to 5% B). The title compound was attained as a pale-yellow solid (3.20 g, 90%). ¹H NMR (400 MHz, CDCl₃,298 K) 7.89 (2H, s, 2-CH), 4.87 (2H, d, ⁴J=2.4 Hz, 3-CH₂), 4.01 (6H, s, 1-CH₃), 2.62 (1H, t, ⁴J=2.4 Hz, 4-CH). ¹³C {¹H} NMR (75 MHz, CDCl₃,298 K) 165.7 (2-C), 165.1 (5-C), 150.0 (3-C), 115.0 (4-C), 77.7 (7-C), 76.3 (8-C), 56.5 (6-C), 53.4 (1-C).

Methyl (6-hydroxymethyl)-4-(prop-2-yn-1-yloxy)picolinate (S5) NaBH₄ (190 mg, 4.92 mmol) was added in small portions to a solution of compound 2 (1.22 g, 4.90 mmol) in dry MeOH/CH₂Cl₂ (40 mL, 1:1) at 0° C. with vigorous stirring. The reaction mixture was stirred for 30 minutes at 0° C., then allowed to warm to RT and stirred for a further 3.5 h. Upon completion, the reaction solution was cooled to 0° C. and quenched with de-ionised H₂O (25 mL). The volatiles were removed in vacuo and the resulting aqueous phase extracted with CH₂Cl₂ (3×30 mL). The combined organic phase was dried over Na₂SO₄, filtered and evaporated in vacuo to give an off-white solid. The crude material was purified via silica gel chromatography (CombiFlash automated purification system; A: CH₂Cl₂, B: MeOH; 100% A to 5% B) to give the title compound as a white crystalline solid (721 mg, 67%). ¹H NMR (400 MHz, CDCl₃, 298 K) 7.57 (1H, d, ⁴J=2.4 Hz, 2-CH), 7.16 (1H, d, ⁴J=2.4 Hz, 5-CH), 4.79 (2H, s, 6-CH₂), 4.78 (2H, d, ⁴J=3-CH₂), 4.22 (1H, br s, 7-OH), 3.93 (3H, s, 1-CH₃), 2.61 (1H, t, ⁴J=2.4 Hz, 4-CH). ¹³C {1H} NMR (100 MHz, CDCl₃,298 K) 165.4 (2-C), 165.2 (5-C) (7-C), 162.9 (-C), 148.6 (3-C), 111.2 (4-C), 109.9 (6-C), 77.1 (10-C), 76.8 (11-C), 64.7 (8-C), 56.0 (9-C), 53.0 (1-C).

Methyl 6-(bromomethyl)-4-(prop-2-yn-1-yloxy)picolinate (S1)Phosphorus tribromide (85 mL, 245 mg, 0.905 mmol, 1.1 equiv.) was added dropwise to a cooled solution of methyl 6-(hydroxymethyl)-4-(prop-2-yn-1-yloxy)picolinate (182 mg, 0.823 mmol) in dry CHCl₃ (5 mL). The reaction mixture was warmed passively to RT and stirred for 3 h. Upon completion, the yellow reaction mixture was quenched with sat. NH₄Cl (20 mL) and extracted with CH₂Cl₂ (4×20 mL). The combined organic phase was evaporated in vacuo to yield the title compound as a white solid (230 mg, 99%). ¹H NMR (300 MHz, CDCl₃,298 K) 7.63 (1H, d, ⁴J=2.4 Hz, 2-CH), 7.22 (1H, d, ⁴J=2.4 Hz, 5-CH), 4.79 (2H, d, ⁴J=2.4 Hz, 3-CH), 4.56 (2H, s, 6-CH₂), 3.96 (3H, s, 1-CH₃), 2.60 (1H, t, ⁴J=2.4 Hz, 4-CH). ¹³C {¹H} NMR (75 MHz, CDCl₃, 298 K) 165.4 (2-C), 165.3 (5-C), 159.1 (7-C), 149.4 (3-C), 113.7 (6-C), 111.7 (4-C), 77.5 (10-C), 76.5 (11-C), 56.2 (9-C), 53.3 (1-C), 33.3 (8-C). ESI-MS (MeOH) 284.0.

tert-butyl (4-(2-hydroxyethyl)phenyl)carbamate (S6)Di-tert-butyl dicarbonate (3.50 g, 16.0 mmol, 1.1 equiv.) was added to a solution of 2-(4-aminophenyl)ethanol (2.01 g, 14.6 mmol) and triethylamine (2.0 mL, 1.46 g, 14.6 mmol) in dry THE (50 mL). The reaction mixture was stirred overnight at RT, then heated to 50° C. for a further 5 h. Upon completion, the volatiles were removed in vacuo to give the title product as an off-white solid (3.35 g, 97%) which was used without further purification. H NMR (300 MHz, CDCl₃,298 K) 7.29 (2H, d, J=8.4 Hz, 3-CH), 7.13 (2H, d, J=8.4 Hz, 4-CH), 6.49 (1H, br s, 2-NH), 3.80 (2H, t, J=6.6 Hz, 6-CH₂), 2.80 (2H, t, J=6.6 Hz, 5-CH₂), 1.50 (9H, s, 1-C(CH₃)₃). ¹³C {1H}NMR (75 MHz, CDCl₃,298 K) 153.0 (3-C), 136.9 (4-C), 133.2 (7-C), 129.7 (6-C), 119.1 (5-C), 80.6 (2-C), 63.8 (9-C), 38.6 (8-C), 28.5 (1-C). ESI-MS (MeOH) 260.2 [M+Na]⁺.

tert-butyl (4-(2-azidoethyl)phenyl)carbamate (S2). Methanesulphonyl chloride (825 mL, 1.22 g, 10.6 mmol, 1.1 equiv.) was added dropwise to a solution of tert-butyl (4-(2-hydroxyethyl)phenyl)carbamate (2.29 g, 9.68 mmol) and DIPEA (1.85 mL, 1.37 g, 10.6 mmol, 1.1 equiv.) in dry EtOAc (20 mL) at 0° C. The solution was stirred for 10 min over ice, then warmed to RT and stirred for a further 1 h. Upon completion, the reaction mixture was cooled to 0° C., filtered, and the precipitate washed with cold EtOAc (70 mL). The filtrate was concentrated in vacuo to yield corresponding mesylate as a pale-yellow oil (S7), which was used without further purification. ¹H NMR (300 MHz, CDCl₃,298 K) 7.31 (2H, d, J=8.5 Hz, 3-CH), 7.12 (2H, d, J=8.5 Hz, 4-CH), 6.67 (1H, br s, 2-NH), 4.34 (2H, t, J=7.1 Hz, 6-CH₂), 2.96 (2H, t, J=7.1 Hz, 5-CH₂), 2.83 (3H, s, 7-CHs), 1.49 (9H, s, 1-C(CHs)₃). ¹³C {¹H} NMR (75 MHz, CDCl₃,298 K) 152.9 (3-C), 137.5 (4-C), 130.8 (7-C), 129.6 (6-C), 118.9 (5-C), 80.5 (2-C), 70.5 (9-C), 37.4 (10-C), 35.0 (8-C), 28.4 (1-C). R_{f}=0.75 (Hexanes/EtOAc, 1:1). 4-((tert-butoxycarbonyl)amino)phenethyl methanesulfonate (S7) (3.03 g, 9.66 mmol) was dissolved in dry DMF (15 mL) and NaN₃ (703 mg, 10.6 mmol, 1.1 equiv.) added. The reaction mixture was heated to 90° C. and stirred overnight. Upon completion, the volatiles were removed in vacuo and the resulting residue partitioned between de-ionised H₂O (50 mL) and EtOAc (50 mL). The aqueous phase was further extracted with EtOAc (2×50 mL), and the combined organic phase evaporated in vacuo to yield the title compound as a pale-yellow oil (2.18 g, 86%). ¹H NMR (300 MHz, CDCl₃, 298 K) 7.30 (2H, d, ³J=8.4 Hz, 3-CH), 7.08 (2H, d, ³J=8.4 Hz, 2-CH), 6.91 (1H, br s, 2-NH), 3.42 (2H, t, ³J=7.2 Hz, 5-CH₂), 2.79 (2H, t, ³J=7.2 Hz, 6-CH₂), 1.48 (9H, s, 1-C(CH₃)₃). ¹³C {¹H} NMR (75 MHz, CDCl₃, 298 K) 153.0 (3-C), 137.4 (4-C), 132.5 (7-C), 129.3 (6-C), 118.9 (5-C), 80.4 (2-C), 52.6 (9-C), 34.8 (8-C), 28.4 (1-C). ESI-MS (MeOH) 285.16 [M+Na]⁺. R_{f}=0.95 (Hexanes/EtOAc, 1:1).

### Example 8.0—Radiolabelling Studies of Bifunctional H₄noneunpaX-Bn-NH₂

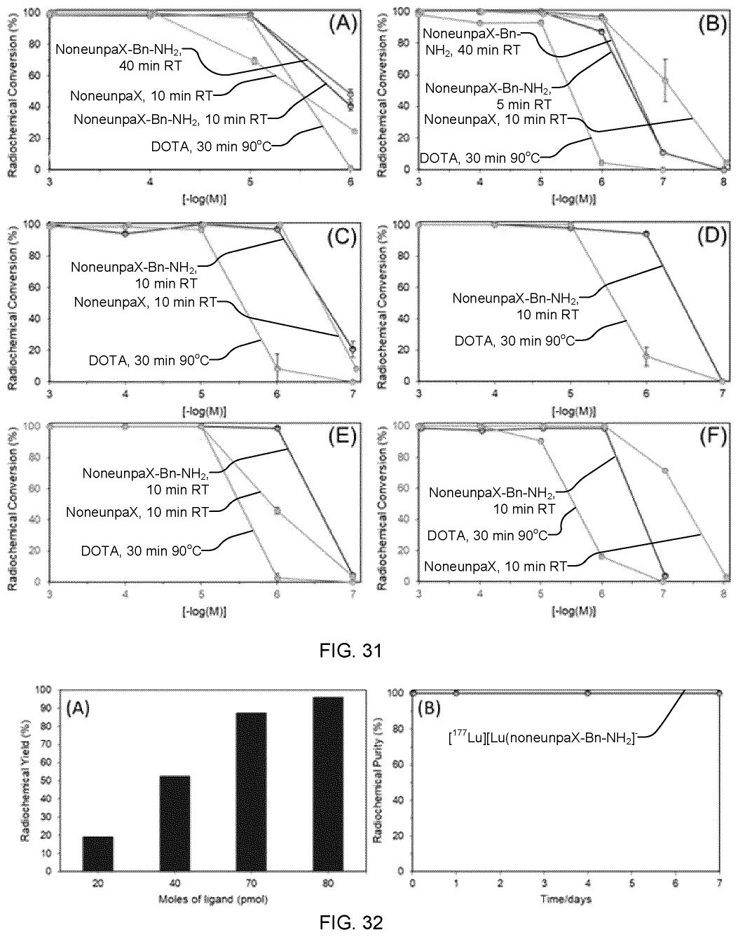

Concentration-dependent radiolabelling studies of H₄noneunpaX-Bn-NH₂ were undertaken to assess the impacts of bifunctionalisation through one picolinic acid donor group on the radiometal ion chelation, FIG. 31. All reactions were conducted at ambient temperature and radiochemical yields (RCYs) determined via iTLC using SA-paper plates and EDTA (50 mM, pH 5.0) as eluent (n=4), except in the case of [²²⁵Ac]Ac³⁺, which used EDTA (50 mM, pH 7.0) as TLC eluent.

Radiolabelling of H₄noneunpaX-Bn-NH₂ with [⁴⁴Sc]Sc³⁺ (400 kBq) demonstrated comparable results to the original chelating ligand, achieving quantitative RCC within 10 min. at RT, thus implying no significant changes to the coordination environment were incurred through functionalisation using this approach. Without being bound by theory, the moderate improvement in RCC at low ligand concentration (10⁻⁵ M) may be attributed to the bifunctional appendage imposing a degree of preorganisation in the binding cavity, thereby favouring metal complexation under these conditions.

Radiolabelling studies of H₄noneunpaX-Bn-NH₂ with [¹⁷⁷Lu]Lu³⁺ also showed comparable results to the unmodified chelator; achieving quantitative RCYs over a wide concentration range (10⁻³ to 10⁻⁶ M) at RT within 10 minutes. Further screening of the radiolabelling properties of H₄noneunpaX-Bn-NH₂ with [¹¹¹In]In³⁺, [¹⁷⁷Lu]Lu³⁺, [^{133/135}La]La³⁺, [¹⁵⁵Tb]Tb³⁺ and [²²⁵Ac]Ac³⁺ showed comparable results to the unmodified chelator, with quantitative RCCs being achieved at ligand concentrations of 10⁻⁶ M with each respective radiometal ion. Notably, the amount of radioactivity used for assessment of RCCs with each radionuclide corresponds to similar molar equivalents of radiometal ions thereby allowing more direct comparison of the concentration-dependence with different radionuclides.

In addition, an evaluation of the maximum molar activity of [¹⁷⁷Lu][Lu(noneunpaX-Bn-NH₂)]⁻ was conducted to determine minimal amount of chelate required to achieve quantitative RCYs using 20 MBq of [¹⁷⁷Lu]Lu³⁺, FIG. 32 left panel. Quantitative radiolabelling of [¹⁷⁷Lu]Lu³⁺ (20 MBq) was achieved using 80 μmol of H₄noneunpa-Bn-NH₂, corresponding to a molar activity of 250 GBq/μmol and a ligand-to-metal ratio of 174:1. Human serum stability studies of [¹⁷⁷Lu][Lu(noneunpaX-Bn-NH₂)]⁻ showed no transchelation of bound radioactivity over 7 days, thereby confirming that modification of the pendent donor arm does not impact the overall stability of the metal complex, FIG. 32 right panel.

### Example 9.0—Bioconjugate Studies

As a proof of concept to assess the suitability of H₄noneunpaX for in vivo applications, two structural analogues of H₄noneunpaX-Bn-NCS were prepared incorporating the SSTR2-targeting peptide Tyr³-Octreotate (Tyr³-TATE) for evaluation in mice bearing AR42J exocrine/pancreatic tumour xenografts. Tyr³-TATE was selected as an appropriate model targeting vector due to its ubiquitous use for targeting neuroendocrine tumours (NETs), and to allow direct comparison to existing clinically applied radiotracers, namely [¹⁷⁷Lu][Lu(DOTATATE)].¹⁵

### Synthesis of H₄noneunpaX-Ahx/PEG₂-Tyr³-TATE

Synthesis of the linear resin-bound Tyr³-Octreotate peptides was carried out using standardised Fmoc-based semi-automated solid-phase peptide synthesis (SPPS) on pre-loaded Wang resin, using an analogous approach to that reported by Noor et al.³⁸ The resin-bound linear peptide was prepared according to the sequence: [DPhe-Cys(Acm)-Tyr(tBu)-DTrp(tBu)-Lys(Boc)-Thr(tBu)-Cys(Acm)-Thr(tBu)-OH], Scheme 5. After generation of the resin-bound octapeptide sequence, Nσ-Fmoc-Ahx-CO₂H or Nα-Fmoc-PEG-CO₂H were incorporated as covalent linkers to give the Fmoc-protected linear-peptides (14) and (15) respectively. Cyclisation of the linear peptides was achieved by treatment with iodine in DMF, which sequentially removes the acetamido methyl (Acm) protecting groups and mediates the formation of the disulfide bridge between Cys² and Cys⁷. Global deprotection and cleavage from the resin was carried out using a standardised TFA cleavage cocktail to give the cyclised Fmoc-protected peptides (16) and (17) (Fmoc-Ahx-Tyr³-TATE (16) and Fmoc-PEG₂-Tyr³-TATE (17)), which were isolated by RP-HPLC. ESI-MS (H₂O/MeCN): Fmoc-Ahx-Tyr³-TATE 1384.4 [M+H]⁺, 1382.7 [M−H]⁻; Fmoc-PEG₂-Tyr³-TATE 1416.8 [M+H]⁺, 1414.7 [M−H]⁻.

To allow selective functionalisation of the N-terminus of each peptide, the side chain primary amine group on Lys⁵ was firstly protected using di-tert-butyl dicarbonate prior to N^{α}-Fmoc cleavage with 20% piperidine in DMF to give compounds (18) and (19). This approach was selected to maximise the purity of the Tyr³-Octreotate peptides prior to incorporating the bifunctional chelator. ESI-MS (H₂O/MeCN): H₂N-Ahx-Boc(Lys⁵)-Tyr³-TATE (18) 1262.9 [M+H]⁺, 1260.8 [M−H]⁻; H₂N-PEG₂-Boc(Lys⁵)-Tyr³-TATE (19) 1294.8 [M+H]⁺, 1292.7 [M−H]⁻.

Conjugation of H₄noneunpaX-Bn-NCS to the free N-terminus of (18) and (19) was achieved under mild, basic conditions in solution, and final cleavage of N(Boc)-Lys⁵ protecting group gave the corresponding chelate-peptide bioconjugates (20) and (21). H₄noneunpaX-Ahx-Tyr³-TATE (20) and H₄noneunpaX-PEG₂-Tyr³-TATE (21) were purified via RP-HPLC and mass spectrometry analysis (ESI/MALDI) performed to confirm isolation of the intended products.

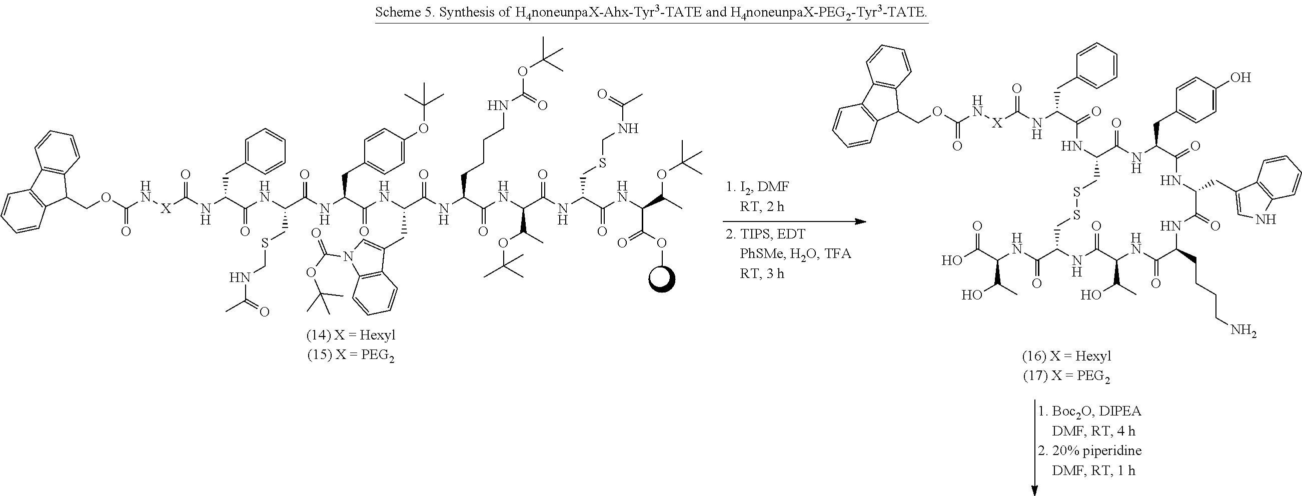

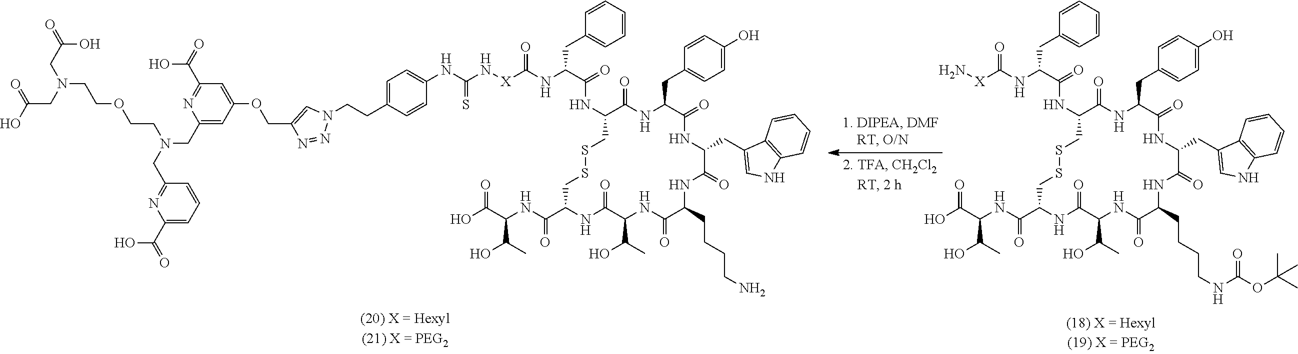

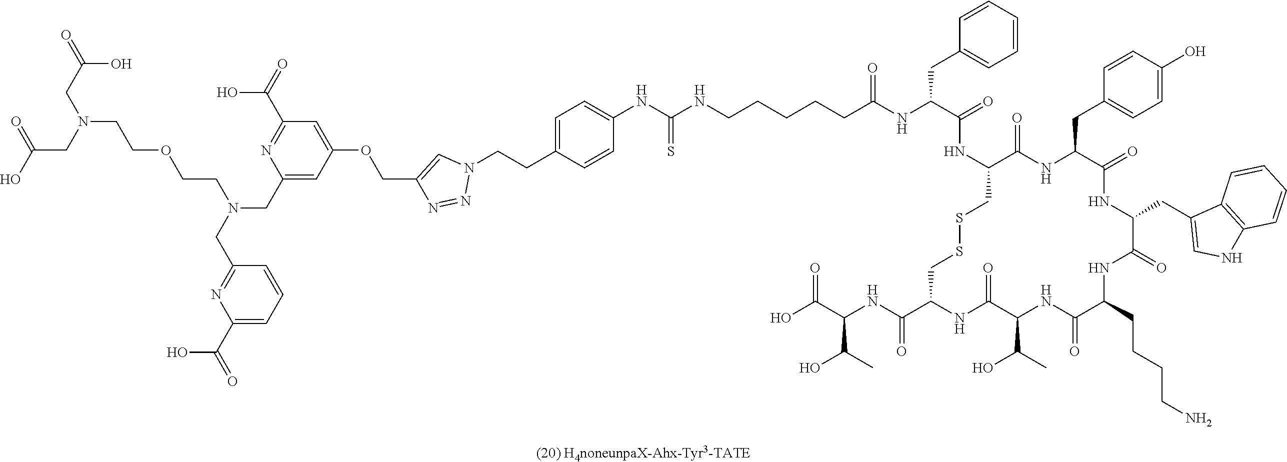 [SMILES: CC(O)[C@H](NC(=O)[C@@H]1CSSC[C@H](NC(=O)[C@@H](Cc2ccccc2)NC(=O)CCCCCNC(=S)Nc2ccc(CCn3cc(COc4cc(CN(CCOCCN(CC(=O)O)CC(=O)O)Cc5cccc(C(=O)O)n5)nc(C(=O)O)c4)nn3)cc2)C(=O)N[C@@H](Cc2ccc(O)cc2)C(=O)N[C@H](Cc2c[nH]c3ccccc23)C(=O)N[C@@H](CCCCN)C(=O)N[C@@H](C(C)O)C(=O)N1)C(=O)O]

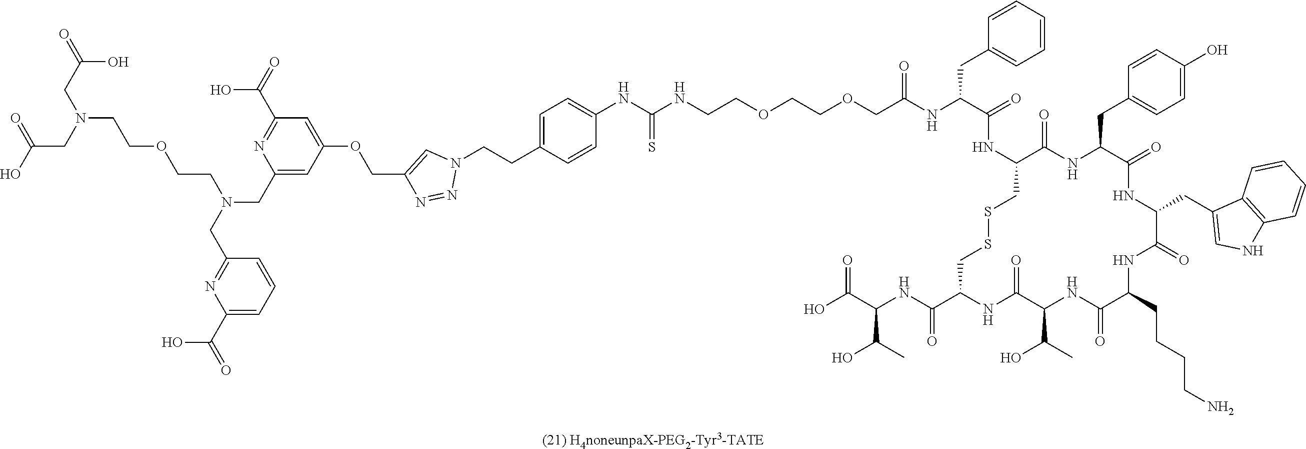 [SMILES: CC(O)[C@H](NC(=O)[C@@H]1CSSC[C@H](NC(=O)[C@@H](Cc2ccccc2)NC(=O)COCCOCCNC(=S)Nc2ccc(CCn3cc(COc4cc(CN(CCOCCN(CC(=O)O)CC(=O)O)Cc5cccc(C(=O)O)n5)nc(C(=O)O)c4)nn3)cc2)C(=O)N[C@@H](Cc2ccc(O)cc2)C(=O)N[C@H](Cc2c[nH]c3ccccc23)C(=O)N[C@@H](CCCCN)C(=O)N[C@@H](C(C)O)C(=O)N1)C(=O)O]

### Synthesis of H₄noneunpaX-Ahx-Tyr³-TATE

In more detail, H₄noneunpaX-Bn-NCS (3.7 mg, 4.95 μmol) was added to a solution of H₂N-Ahx-Boc(Lys⁶)-Tyr³-TATE (6.3 mg, 4.95 μmol) in dry DMF (1 mL). The solution was stirred for 10 min. at RT then DIPEA (10 μL, 57 μmol) added. The resulting mixture was stirred O/N at RT, then treated with 20% TFA in CH₂Cl₂ (1 mL) and stirred for a further 2 h. After completion, the volatiles were evaporated under a stream of N₂ gas, and the resulting residue diluted with H₂O (0.1% TFA). The crude bioconjugate was purified via semipreparative RP-HPLC (A: H₂O (0.1% TFA), B: MeCN (0.1% TFA) (3 mL/min); method: 0-6 min. (95% A to 29% B); 6-25 min. (ISO 29% B); 25-30 min. (29% B to 100% B); Rₜ=22.6 min. Appropriate fractions were pooled and lyophilised to give H₄noneunpaX-Ahx-Tyr³-TATE (20) as a white solid. ESI-MS (H₂O/MeCN (1:1)) 1911.9 [M+H]⁺.

### Synthesis of H₄noneunpaX-PEG₂-Tyr³-TATE

H₄noneunpaX-Bn-NCS (2.3 mg, 3.07 μmol) was added to a solution of H₂N-PEG₂-Boc(Lys⁶)-Tyr³-TATE (4.0 mg, 3.07 μmol) in dry DMF (500 μL). The solution was stirred for 10 min. at RT then DIPEA (10 μL, 57 μmol) added. The resulting mixture was stirred O/N at RT, then treated with 20% TFA in CH₂Cl₂ (1 mL) and stirred for a further 2 h. After completion, the volatiles were evaporated under a stream of N₂ gas, and the resulting residue diluted with H₂O (0.1% TFA). The crude bioconjugate was purified via semipreparative RP-HPLC (A: H₂O (0.1% TFA), B: MeCN (0.1% TFA) (3 mL/min); method: 0-6 min. (95% A to 29% B); 6-25 min. (ISO 29% B); 25-30 min. (29% B to 100% B); Rₜ=19.1 min. Appropriate fractions were pooled and lyophilised to give H₄noneunpaX-PEG₂-Tyr³-TATE (21) as a white solid. ESI-MS (H₂O/MeCN (1:1)) 1943.3 [M+H]⁺, 972.7 [M+2H]²⁺

### Radiolabelling Studies of Chelate-Bioconjugates

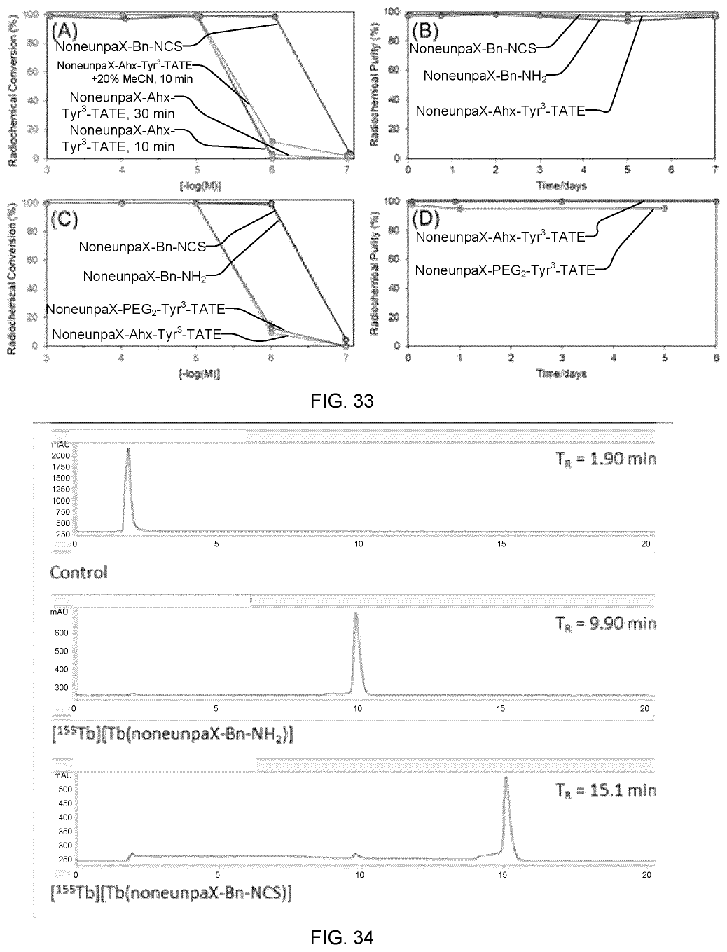

The concentration-dependent radiolabelling of H₄noneunpaX-Ahx-Tyr³-TATE and H₄noneunpaX-PEG₂-Tyr³-TATE was evaluated with [¹⁵⁵Tb]Tb³⁺ and [²⁵⁵Ac]Ac³⁺ as depicted in FIG. 33. Each of the chelate-bioconjugates attained quantitative RCCs at a concentration of 10⁻⁵ M within 10 min at ambient temperature. The decrease in RCC at lower concentrations in comparison to the free bifunctional chelator is typical for peptide-based bioconjugates, which is attributed to the steric influence of the targeting vector on the metal binding cavity, in addition to the lower solubility of the constructs in aqueous solution and thus availability for metal ion coordination. Assessment of the serum stability for each of the radiolabelled bioconjugates showed excellent stability over the course of the study, with no significant changes in radiochemical purity from the initial time-points.

For concentration-dependent radiolabelling studies, the following general protocol was applied for radiolabelling with different radiometal ions. Stocks solution of each respective chelating ligand were prepared in ultra-pure de-ionised H₂O at a concentration of 1×10⁻² M. Serial dilution series for each chelator were prepared prior to radiolabelling studies, over a concentration range of 10⁻³ to 10⁻⁶ M. Aliquots (10 μL) of each stock solution were added to NaOAc (0.1 M), NH₄OAc (0.5 or 1.0 M) or MES buffer (1.0 M) (90 μL) to give reaction solutions suitable for study. Aliquots (1-10 μL) of each respective radionuclide were added, under the following conditions: [⁴⁴Sc]Sc³⁺ (1.2 MBq) in NaOAc (0.1 M, pH 4.5), [¹¹¹In]In³⁺ (1.0 MBq) in NH₄OAc (0.5 M, pH 5.8), [^{132/135}La]La³⁺ (400 kBq) in NH₄OAc (0.2 M, pH 7.0), [¹⁵⁵Tb]Tb³⁺ (40 kBq) in NH₄OAc (0.5 M, pH 6.0), [¹⁷⁷Lu]Lu³⁺ (150 kBq) in NH₄OAc (0.5 M, pH 6.0), [²¹³Bi]Bi³⁺ (680 kBq) in MES (1.0 M, pH 5.5), [²²⁵Ac]Ac³⁺ (40 kBq) in NH₄OAc (1.0 M, pH 7.3). Reactions were carried out at RT and monitored over 10 minutes, with the exception [²¹³Bi]Bi³⁺ which were monitored after 5 minutes, within 10 minutes post-generator elution (n=4-8). Reactions with DOTA were carried out at elevated temperatures (85-90° C.) and monitored over 30-60 minutes. Radiochemical yields (RCYs) determined via iTLC using SA-paper plates and EDTA (50 mM, pH 5.0 or 7.0) as eluent (n=4). In the case of [²¹³Bi]Bi³⁺ RCYs were further confirmed through gamma spectroscopy measurements by analysis of the baseline and solvent front TLC peaks using a high-purity germanium (HPGe) detector and monitoring the 440 keV gamma emission line of ²¹³Bi. In all studies, separate control reactions were performed in parallel by addition of radioactivity to solutions containing buffer (90 μL) and de-ionised H₂O (10 μL).

Human serum stability studies were carried out by addition of each radiolabelled complex or bioconjugate (100 μL) to vials containing human serum albumin (100 μL) and incubating the resulting solutions at 37° C. over 5-7 days. The radiochemical purity (RCP) was determined by iTLC using SA-paper plates and EDTA (50 mM, pH 5.5 or 7.0) as eluent, whereby transchelated radioactivity migrates with the solvent front (R_{f}=1.0) while intact metal complexes remain at the baseline (R_{f}=0). All studies were performed in triplicate, and the average % RCP used for assessment of each compound.

Dose escalation studies of [¹⁷⁷Lu][Lu(noneunpaX-Bn-NH₂)] were carried out by sequential additions of H₄noneunpaX-Bn-NH₂ (2-8 μL, 10⁻⁴ M, 20-80 μmol) to a solution containing [¹⁷⁷Lu]LuCl₃ (20 MBq) in NH₄OAc buffer (40 μL, 0.5 M, pH 6.0). The reaction solution was allowed to stand for 10 min. at RT between each addition, after which the RCY was determined by spotting 1 μL of solution onto SA-paper TLC plates and development with EDTA (50 mM, pH 5.5).

### LogD_{7.4} Measurements

Prior to investigation of the radiotracer performance in vivo, the lipophilicity of the radiolabelled chelate-bioconjugates was determined through measurement of the distribution coefficients between n-octanol and PBS (0.01 M, pH 7.4), Table 5. All four radiolabelled tracers are moderately hydrophilic with logD_{7.4} values ranging between −1.933 to −2.585, which are comparable to those reported for [¹⁶¹Tb][Tb(DOTA-LM3)] (logD_{7.4=-}2.5±0.1), an SSTR2 antagonist analogue of DOTATATE.³⁹ As anticipated, the higher lipophilicity of the aliphatic hexyl covalent linker is reflected in the logD_{7.4} measurements, with both Ahx-Tyr³-TATE tracers being more lipophilic than the PEG₂-Tyr³-TATE derivatives. Notably, interchange between [¹⁵⁵Tb]Tb³⁺ and [²²⁵Ac]Ac³⁺ did not have a significant impact on the logD_{7.4} values for either bioconjugate, which may indicate a comparable biodistribution profile in vivo using this theranostic pair.

Aliquots of [¹⁵⁵Tb]Tb³⁺ or [²²⁵Ac]Ac³⁺ radiolabelled H₄noneunpaX-Ahx-Tyr³-TATE or H₄noneunpaX-PEG₂-Tyr³-TATE (10 μL) were added to a biphasic mixture of n-octanol (700 μL) and PBS (700 μL, pH 7.4). The mixtures were vortexed for 2 min at RT, and then separated via centrifuge (10 min, 3000 RPM). Aliquots of n-octanol (100 μL) and PBS (100 μL) were collected, and the activity in each portion determined via gamma spectroscopy. LogD_{7.4} measurements were carried out 5-6 replicates per radiotracer. The LogD_{7.4} is defined as: log₁₀ [(n-octanol phase)/(buffer phase)].

TABLE 5
Distribution coefficients (LogD_{7.4}) for H₄noneunpaX-
Ahx-Tyr³-TATE and H₄noneunpaX-PEG-Tyr³-TATE
labelled with [²²⁵Ac]Ac³⁺ and [¹⁵⁵Tb]Tb³⁺,
measured between PBS and n-octanol (n = 5-6).

| Compound | LogD_{7.4} |
|---|---|
| [²²⁵Ac][Ac(noneunpaX-Ahx-Tyr³-TATE)] | −1.933 ± 0.079 |
| [²²⁵Ac][Ac(noneunpaX-PEG₂-Tyr³-TATE)] | −2.507 ± 0.095 |
| [¹⁵⁵Tb][Tb(noneunpaX-Ahx-Tyr³-TATE)] | −2.006 ± 0.061 |
| [¹⁵⁵Tb][Tb(noneunpaX-PEG₂-Tyr³-TATE)] | −2.585 ± 0.042 |

### In Vivo SPECT/CT and Biodistribution Studies

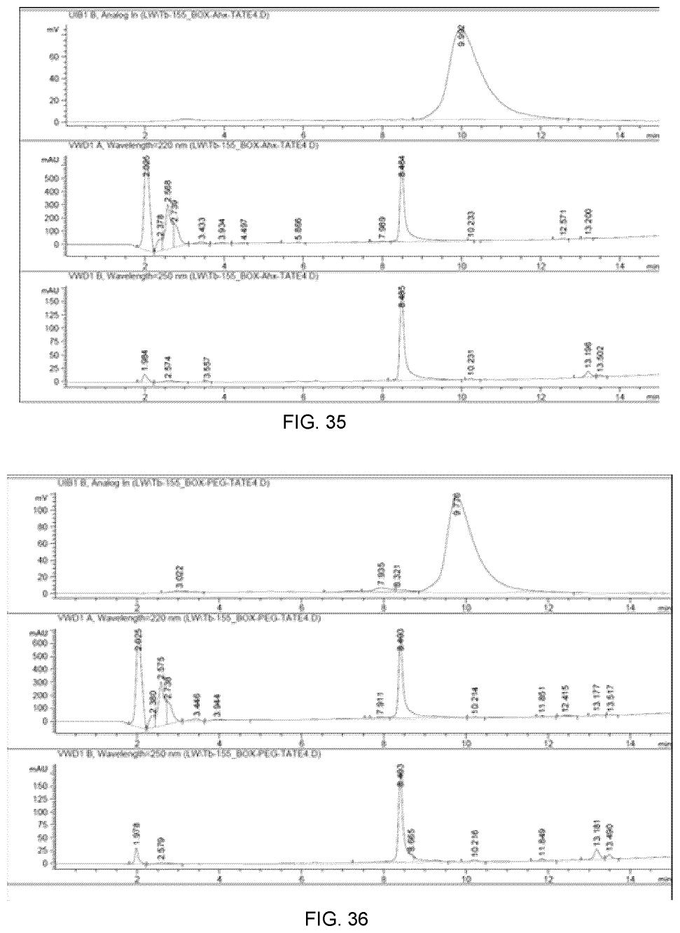

In vivo SPECT/CT studies of [¹⁵⁵Tb]Tb³⁺-labelled H₄noneunpaX-Ahx-Tyr³-TATE and H₄noneunpaX-PEG₂-Tyr³-TATE were performed in NRG mice bearing AR42J tumour xenografts to assess the suitability of [¹⁵⁵Tb]Tb³⁺ as an imaging companion to [²²⁵Ac]Ac³⁺. Each [¹⁵⁵Tb]Tb³⁺-labelled radiotracer was successfully prepared at high molar activity (˜23.6 MBq/nmol) with a high radiochemical purity (>98%) as confirmed by iTLC and radio-HPLC (FIGS. 35-37). For in vivo SPECT/CT studies, mice were administered with 13.5 MBq (0.572 nmol) of each radiotracer, while biodistribution studies were performed using 0.80 MBq (0.033 nmol) per subject.

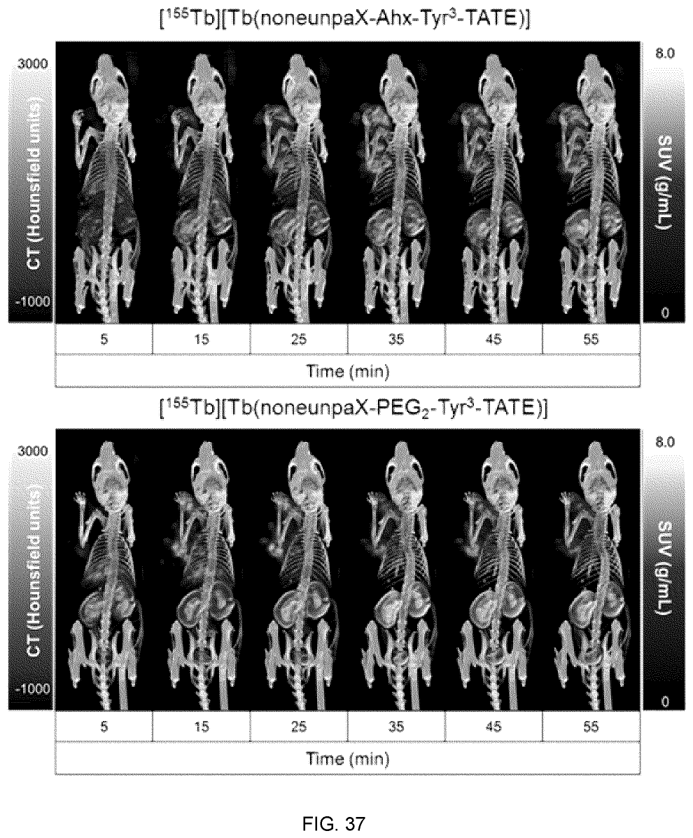

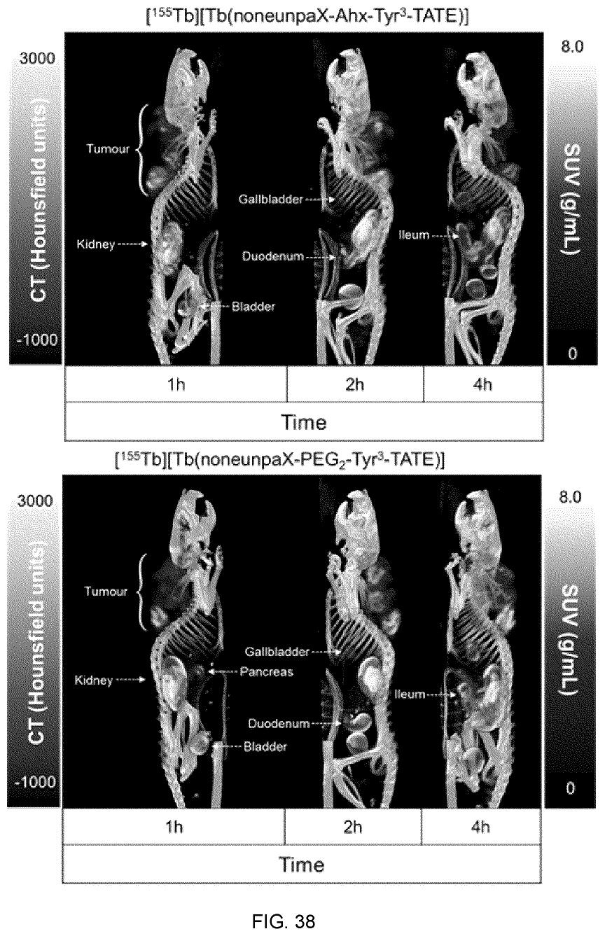

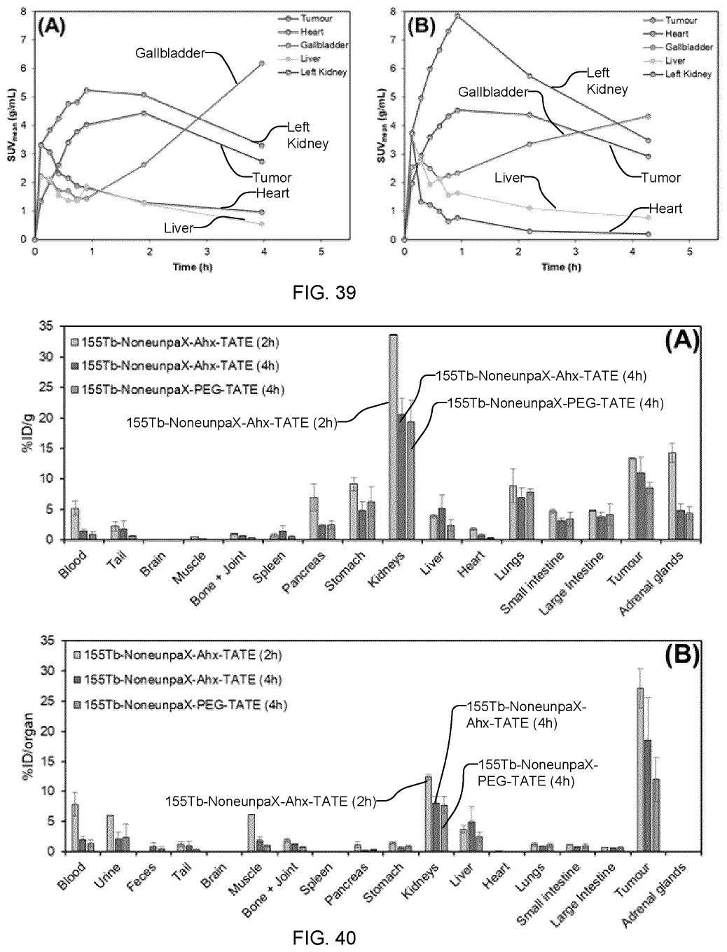

Dynamic SPECT/CT scans of mice bearing AR42J tumour xenografts were acquired over a 1 h period to assess the pharmacokinetics profile of both [¹⁵⁵Tb]Tb-labelled radiotracers after intravenous administration, FIG. 37. Further static SPECT/CT images were recorded at 2 h and 4 h post-administration, and standardised uptake values (SUVs) for regions of interest (ROIs) were extracted from the quantitative image scans to generate time-activity curves for each radiotracer, FIGS. 38 and 39.

In both cases, the [¹⁵⁵Tb]Tb³⁺-labelled radiotracers exhibit rapid clearance from blood circulation over the first 1 h after administration, with uptake in the tumour beginning within the first 5 minutes p.i. Both tracers followed the typical pharmacokinetic profile observed for hydrophilic octreotate-based bioconjugates, whereby fast clearance and accumulation in the kidneys and bladder is seen.^{40,41} Comparatively, both tracers exhibited very similar overall distribution over the course of the study, with [¹⁵⁵Tb][Tb(noneunpaX-PEG₂-Tyr³-TATE)] showing a marginally faster accumulation in the AR42J tumour xenografts, which is accompanied by faster clearance over time compared to [¹⁵⁵Tb][Tb(noneunpaX-Ahx-Tyr³-TATE)]. This observation is clearly in line with the expected trend, accounting for the difference lipophilicity between these bioconjugates. Notably, good contrast in the tumour region is seen after 45 min p.i. with maximum uptake peaking at 2 h p.i.

Clearance of both radiotracers occurs predominantly through the renal pathway, as observed by high uptake in the kidneys and bladder, and low uptake in the liver. The SPECT/CT images show additional clearance via the biliary tract, whereby an increase in activity is seen in the gallbladder beginning at 2 h p.i., with subsequent uptake in the small intestines (ileum, duodenum) at later time-points. This additional elimination pathway may account for the sustained tumour uptake over 1-2 h, in contrast to [¹⁷⁷Lu][Lu(DOTATATE)],⁴⁰ due to reabsorption of the radiotracers in the gastrointestinal tract (enterohepatic circulation) which is commonly seen for drugs exhibiting clearance in the bile.⁴² From assessment of the time-activity curves, the differences in hydrophilicity of each tracer are clearly reflected in the pharmacokinetic profiles; with [¹⁵⁵Tb][Tb(noneunpaX-PEG₂-Tyr³-TATE)] showing higher renal excretion, while [¹⁵⁵Tb][Tb(noneunpaX-Ahx-Tyr³-TATE)] shows greater gallbladder uptake.

SSTR2 is expressed at low levels in normal healthy tissues, including the pancreas, lungs, stomach, adrenal glands, and kidneys, which are clearly visualised in the SPECT/CT images and biodistribution data determined for each tracer, FIG. 40. However, significantly lower accumulation was observed in the pancreas (2.39±0.18% ID/g and 2.43±0.68% ID/g), lungs (6.93±1.60% ID/g and 7.91±0.47% ID/g) and adrenal glands (4.84±1.11% ID/g and 6.28±1.12% ID/g) at 4 h post-injection for [¹⁵⁵Tb][Tb(noneunpaX-Ahx-Tyr³-TATE)] and [¹⁵⁵Tb][Tb(noneunpaX-PEG₂-Tyr³-TATE)] respectively, compared to [¹⁷⁷Lu][Lu(DOTATATE)][pancreas (10.9% ID/g), lungs (17.4% ID/g) and adrenal glands (8.95% ID/g)].⁴⁰ While higher uptake in the kidneys (19.3±2.78% ID/g and 20.5±3.57% ID/g) was seen compared to [¹⁷⁷Lu][Lu(DOTATATE)] (6.31% ID/g),⁴⁰ the biodistribution study at 2 h p.i. for [¹⁵⁵Tb][Tb(noneunpaX-Ahx-Tyr³-TATE)] and time-activity curves for both tracers shows good clearance rather than retention in the kidneys.

The relatively low uptake in the AR42J tumours (11.0±2.62% ID/g and 8.51±0.90% ID/g) at 4 h post-administration is primarily a result of the large tumour size used in these studies. As can be visualised from the SPECT/CT images, the tumours appear non-homogeneous, with regions of necrotic tissue and no radiotracer uptake; hence, the % ID/g in the tumours is likely lower than achievable. However, the primary objective of these studies was to evaluate the in vivo suitability of H₄noneunpaX as a new bifunctional chelating ligand. Overall, the imaging and biodistribution studies show good initial results for this chelator, with no evidence of degradation in vivo or release of bound activity, which would be seen as uptake in the bone over time. In addition, both Tyr³-TATE analogues show improved pharmacokinetics with lower non-target tissue accumulation compared to [¹⁷⁷Lu][Lu(DOTATATE)] and would be of interest for further development and evaluation in vivo.

For the foregoing studies, [¹⁵⁵Tb][Tb(nonenunpaX-Ahx-Tyr³-TATE)] and [¹⁵⁵Tb][Tb(nonenunpaX-PEG₂-Tyr³-TATE)] were prepared with high molar activities (23.6 MBq/nmol and 22.5 MBq/nmol, respectively), suitable for in vivo SPECT/CT imaging and biodistribution studies. An aliquot of [¹⁵⁵Tb]Tb³⁺ (28 MBq, 40 μL) was added to a solution of H₄noneunpaX-Ahx-Tyr³-TATE (6 μL, 2×10⁻³ M, 1.2 nmol) in NH₄OAc buffer (10 μL, 0.5 M, pH 5.5). The pH was adjusted to neutral by addition of NaOH (1 M, 1 μL) and the resulting solution incubated at 37° C. for 15 minutes to ensure quantitative incorporation of radioactivity. The same radiolabelling protocol was applied to [¹⁵⁵Tb][Tb(nonenunpaX-PEG₂-Tyr³-TATE)]. Quality control measurements were performed via iTLC measurements and radio-HPLC; method: A: H₂O (0.1% TFA), B: MeCN (0.1% TFA), 100% A to 60% B; 15 min, 1 mL/min., [¹⁵⁵Tb][Tb(nonenunpaX-Ahx-Tyr³-TATE)] (t_{R}=9.99 min., 99%), [¹⁵⁵Tb][Tb(nonenunpaX-PEG₂-Tyr³-TATE)] (t_{R}=9.78 min., 96%) (FIGS. 35 and 36). A small aliquot of each radiolabelled tracer was taken for quantification of radioactivity using gamma spectroscopy. No further purification was performed prior to administration of either radiotracer. Each of the radiolabelled bioconjugates was divided into two different stocks and diluted with PBS, to provide doses suitable for SPECT/CT imaging (10.2-13.5 MBq per subject) or biodistribution studies (˜800 kBq per subject).

Tumour implantation was performed at the BCCRC under the protocol approved by the Animal Care Committee (ACC) of the University of British Columbia (A20-0113). Female NRG mice were anesthetized by inhalation with 2% isoflurane in 2.0 L/min of oxygen and inoculated with a AR42J exocrine pancreatic tumour cell line, subcutaneously on the left shoulder. In vivo imaging and biodistribution studies were performed after tumour growth reached ˜8-10 mm in diameter (2-3 weeks post inoculation).

Animal studies were performed in accordance with the Canadian Council on Animal Care (CCAC) using the protocol approved by the Animal Care Committee (ACC) of the University of British Columbia (A20-0132). Female NRG mice bearing AR42J exocrine pancreatic tumour xenografts were anesthetized with 5% isoflurane in an induction chamber and restrained in a tail vein restrainer (Braintree Scientific) while under a continuous stream of 1-1.5% isoflurane. Mice were administered with either [¹⁵⁵Tb][Tb(noneunpaX-Ahx-Tyr³-TATE (10.2 MBq, 23.6 MBq/nmol) or [¹⁵⁵Tb][Tb(noneunpaX-PEG₂-Tyr³-TATE (13.5 MBq, 22.5 MBq/nmol) in PBS (100 μL) via lateral tail vein. Animal subjects were maintained at constant body temperature using a blanket on a heated bed, maintained under continuous stream of 1.5-2% isoflurane, and the respiration rate monitored throughout the duration of each scan. Immediately after injection, whole-body dynamic SPECT/CT imaging scans were acquired over the first 60 min using a multimodal VECTor/CT system (MILabs, Netherlands) equipped with an extra ultra high sensitivity (XUHS) 2-mm pinhole collimator. The mouse whole-body region was centered with a 14 mm axial field of view, and dynamic imaging consisting of six frames of 10 min were acquired over the first 60 min., after which static SPECT/CT scans were recorded at 2 h and 4 h post-injection using single frames of 20 min. acquisitions. Energy windows centred on the 44, 85 and 106 keV photopeaks of ¹⁵⁵Tb were applied, with a spectral width of 25%. For quantitative analysis, the SPECT images were reconstructed using pixel-based ordered-subset expectation maximization (POSEM) reconstruction algorithm using a voxel size of 0.4 mm³, 16 subsets with 6 iterations (96 MLEM equivalent). SPECT images were decay corrected and attenuation factors applied based on CT acquisitions at each time-point.⁵³ A calibration factor relating (counts/voxel) to radioactivity concentration was previously determined by measurement of a known source of ¹⁵⁵Tb. Spherical volumes of interest (VOIs) (3 mm diameter) were drawn using AMIDE (v. 1.0.4) software to determine the pharmacokinetic profile of the tracer in target organs of interest. Average standardised uptake values (SUVs) were subsequently extracted from the SPECT images. The standardised uptake value was defined according to the equation: SUV (g/mL)=radioactivity concentration (MBq/mL)/[administered dose (MBq)/body weight (g)]. Gaussian filtering (FWHM=3 mm) and image rendering was carried out post-reconstruction for data visualisation purposes only.

Biodistribution studies with [¹⁵⁵Tb][Tb(noneunpaX-Ahx-Tyr³-TATE)] and [¹⁵⁵Tb][Tb(noneunpaX-PEG₂-Tyr³-TATE)] were undertaken in NRG mice bearing AR42J exocrine pancreatic tumour xenografts. Prior to administration of each radiotracer (˜800 kBq, 0.033 nmol) in PBS (˜100 μL), mice were anaesthetized by inhalation of 2% isoflurane and restrained using a Tail vein restrainer (Braintree Scientific). Intravenous administration of each radiotracer occurred via the lateral tail vein. After injection, mice were allowed to roam freely in their cages and sacrificed at 2 h or 4 h post-injection by CO₂ asphyxiation under 2% isoflurane anesthesia. Cardiac puncture was performed immediately after sacrifice to recover blood, and organs of interest were harvested, rinsed with PBS and blotted dry. Each organ was weighed, and the radioactivity measured using a calibrated gamma counter (Packard Cobra II Auto-gamma counter, Perkin Elmer, Waltham, MA, USA) with a 1 min acquisition time per sample. All radioactivity measurements were decay corrected to the time of injection, and the injected dose per gram of tissue (% ID/g) calculated based on measured organ weights, with the exception of blood, bone and muscle which were scaled in accordance with literature values.⁵⁴

### General Methods and Materials

All solvents and reagents were purchased from commercial suppliers (Sigma-Aldrich, AK Scientific, Alfa Aesar) and were used directly without further purification. Analytical thin-layer chromatography (TLC) sheets were purchased from Merck (TLC Silica gel 60 F254, aluminium sheet). Deionized H₂O (18.2 MO/cm at 25° C.) was obtained from a PURELAB Ultra water purification system, ELGA LabWater. Flash column chromatography was performed using Siliaflash F60 silica gel (60 Å, 40-63 μm particle size, 230-400 mesh) from Silicycle Inc. Automated column chromatography was performed using a Teledyne Isco (LincoIn, NE) CombiFlash R_{f} automated purification system equipped with RediSep R_{f} Gold HP prepacked reusable silica and neutral alumina column cartridges. Low-resolution mass spectrometry (LR-MS) was performed using a Waters 2965 ZQ spectrometer with an electrospray/chemical ionization (ESI/CI) source. High-resolution mass spectrometry (HR-MS) was performed using a Waters Micromass LCT TOF instrument. Elemental analyses (CHN) were carried out using a Thermoflash 2000 elemental analyzer. ¹H and ¹³C{¹H} NMR spectra were recorded using Bruker AV300 and AV400 spectrometers; all spectra are reported on the delta scale referenced to residual solvent peaks. Analytical and semipreparative high-performance liquid chromatography (HPLC) was carried out using a Waters 600 system equipped with a Waters 2487 dual wavelength absorbance detector monitoring at 254 and 210 nm and a Phenomenex Synergi 250 mm×21.2 mm 4 μm hydro-RP80 Δ column (10 mL/min). All HPLC methods employed a H₂O/MeCN biphasic solvent system buffered with 0.1% TFA. HPLC solvent system 1 (A: H₂O (0.1% TFA), B: MeCN (0.1% TFA), HPLC solvent system 2: (A: H₂O (0.01% TFA), B: MeCN). [⁴⁴Sc]Sc³⁺ (t_{1/2}=3.97 hours) was produced at TRIUMF via proton irradiation of ^{nat}Ca targets with 12.8 MeV protons, as previously reported.²⁸ [¹¹¹In]In³⁺ (t_{1/2}=2.83 days) (purchased from BWX Technologies) was produced by proton irradiation (Advanced Cyclotron Systems, Model TR30) via the ¹¹¹Cd(p,n)¹¹¹In reaction and provided as a 0.05 M HCl solution. [¹⁵⁵Tb]Tb³⁺ (t_{1/2}=5.58 days) was produced at TRIUMF via irradiation of tantalum targets with 500 MeV protons, followed by directed isotope separation online (ISOL) and implantation into NH₄Cl layered aluminium disks.⁴³ [^{132/135}La]La³⁺ (t_{1/2}=19.5 hours) was produced via irradiation of ^{nat}Ba targets with 12.8 MeV protons, using a similar approach to the methods reported by Aluicio-Sarduy et al. ⁴⁴ [¹⁷⁷Lu]Lu³⁺ (t_{1/2}=6.67 days) was obtained from ITM Medical Isotopes GmbH Germany as a 0.05 M HCl solution. [²¹³Bi][Bi³⁺ was obtained from an in-house ²²⁵Ac/²¹³Bi generator constructed using AG-MP-50 cation exchange resin, based on established methodologies.⁴⁵ [²²⁵Ac]Ac³⁺ (t_{1/2}=10.0 days) was produced at TRIUMF via the spallation of ²³²Th targets with 500 MeV protons, and purified as previously reported.⁴⁶ Radiolabelling of compounds was assessed via instant thin-layer chromatography (iTLC) using silicic acid (SA)-impregnated paper TLC plates sourced from Agilent technologies. TLC imaging was performed using an AR-2000 imaging scanner equipped with P-10 gas, and subsequent analysis of radiochemical conversion (RCC) was carried out using WinScan V3_14 software. Radio-HPLC was carried out using an Agilent 1200 instrument equipped with a Phenomenex Synergi 4 μm 250 mm×4.6 mm hydro-RP 80 Δ column. Radioactivity was quantified using a calibrated high-purity germanium (HPGe) detector (Mirion Technologies (Canberra) Inc.) with Genie 2000 software. All work with radionuclides at TRIUMF was undertaken in shielded fume hoods to minimize dose to experimenters (and special precautions were used to prevent contamination). Peptides were prepared using an AAPPTec Focus Xi semi-automated solid phase peptide synthesiser. SPECT/CT studies were performed using a multimodal VECTor/CT system (MILabs, Netherlands) in combination with an extra ultra high sensitivity (XUHS) 2-mm pinhole collimator. Image analysis was performed using AMIDE (v. 1.0.4) software.⁴⁷

### CONCLUSIONS

A new nonadentate chelating ligand, H₄noneunpaX, was synthesised to investigate the influence of donor group arrangement on metal binding characteristics within the ‘NON backbone’. Characterisation of the metal complexation of H₄noneunpaX through NMR spectroscopy, mass spectrometry, radiolabelling, solution thermodynamic stability studies and DFT calculations, showed excellent compatibility with a wide range of large trivalent metal cations, with a particular preference for hard lanthanide ions. The radiolabelling properties of H₄noneunpaX were assessed with [⁴⁴Sc]Sc³⁺, [¹¹¹In]In³⁺, [^{132/135}La]La³⁺, [¹⁵⁵Tb]Tb³⁺, [¹⁷⁷Lu]Lu³⁺, [²¹³Bi]Bi³⁺ and [²²⁵Ac]Ac³⁺, which demonstrated quantitative RCC within 10 minutes at ambient temperature, and achieved notably high molar activities with [¹¹¹In]In³⁺ (54 GBq/μmol), [¹⁵⁵Tb]Tb³⁺ (1.0 GBq/μmol), [¹⁷⁷Lu]Lu³⁺ (2.0 GBq/μmol) and [²²⁵Ac]Ac³⁺ (134 MBq/μmol). The reactivity trends were found to be highly comparable to the structural related chelating ligand, H₄noneunpa, achieving similar RCYs and molar activities. The kinetic inertness of each metal complex was determined using serum challenge studies, which showed excellent in vitro stability (>95% RCP over 5-7 days), with the exception of [²²⁵Ac][Ac(noneunpa)] which showed a reduction in integrity over time (˜10% degradation), while [²²⁵Ac][Ac(noneunpaX) maintained high radiochemical integrity (>99% RCP over 7 days) thereby showing a small influence between chelate structural arrangements on complex stability. Solution thermodynamic stability studies showed the formation of highly stable metal complexes with H₄noneunpaX, whereby a single dominant species was observed over a broad pH range (pH=2.0-11.5) on complexation with the lanthanide series. High stability constants were determined for each of the metal complexes, indicating highly effective metal scavenging ability.

A bifunctional analogue of H₄noneunpaX was prepared using an efficient, versatile/adaptable synthetic approach, and the complexation properties reassessed, showing comparable characteristics to the unmodified chelator. Dose escalation studies of bifunctional H₄noneunpaX-Bn-NH₂ with [¹⁷⁷Lu]Lu³⁺ demonstrated that highly competitive radiolabelling with notably high molar activities (250 GBq/μmol). The bifunctional derivative of H₄noneunpaX was conjugated to two different Tyr³-octreotate analogues for targeting of SSTR2 in NER tumours.

Preliminary in vivo SPECT/CT imaging, pharmacokinetics and biodistribution studies were undertaken with [¹⁵⁵Tb]Tb³⁺-radiolabelled H₄noneunpaX-Ahx-Tyr³-TATE and H₄noneunpaX-PEG₂-Tyr³-TATE in NRG mice bearing AR42J exocrine pancreatic tumour xenografts. Both radiolabelled bioconjugates showed good in vivo performance, with no evident degradation over the course of the study and good tumour uptake (13.3±0.18% ID/g at 2 h). The biodistribution profile of each tracer showed the typical expected clearance profile of SSTR2 targeting tracers (i.e. [¹⁷⁷Lu][Lu(DOTATATE)], with primarily elimination through the renal pathway, with uptake in the kidneys and bladder, followed by elimination in the urine. Notably, lower accumulation was seen in non-target organs expressing SSTR2 (adrenal glands, lungs, pancreas) compared to [¹⁷⁷Lu][Lu(DOTATATE)]. Ultimately, these studies demonstrate H₄noneunpaX as a valuable, new chelating ligand with beneficial characteristics for e.g. [²²⁵Ac]Ac³⁺ applications in targeted alpha therapy, in combination with other suitable radionuclides for diagnostic imaging. The mild conditions required for radiolabelling of H₄noneunpaX are particularly well suited for combination with thermally sensitive antibody-based targeting vectors and thus it can be soundly predicted that there is a high likelihood that H₄noneunpaX will have good utility as a chelator for the targeted in vivo delivery of radiometals.

The objectives of this work were to investigate the influence of donor group substitution about a common backbone and study the impact of inverted group placement on metal ion affinity. H₄noneunpaX was developed to this affect and studied in direct comparison to H₄noneunpa. An inverted arrangement of donor pendent arms was found to give comparable metal ion binding characteristics to the symmetrically derivatised chelating ligand, with the exception of [²¹³Bi]Bi³⁺. High radiochemical yields (RCYs) and molar activities for H₄noneunpaX were achieved with [¹¹¹In]In³⁺, [¹⁵⁵Tb]Tb³⁺, [¹⁷⁷Lu]Lu³⁺, and [²²⁵Ac]Ac³⁺ under mild conditions (RT, 10 min), in addition to demonstrating high kinetic inertness when challenge in human serum. Cold complexation studies and DFT simulations indicate highly versatile metal ion chelation with H₄noneunpaX, with a particular preference for chelation of trivalent lanthanide ions.

To further assess the utility of this approach, a bifunctional analogue of H₄noneunpaX was prepared and radiolabelling studies with [⁴⁴Sc]Sc³⁺ and [¹⁷⁷Lu]Lu³⁺ performed. Structural modification on the pendent donor groups retained the metal chelation characteristics of the original chelating ligand. Given the synthetic accessibility of bifunctional chelating ligands featuring this inverted donor group arrangement (in contrast to symmetrically derivatised bifunctional chelators), further investigation of H₄noneunpaX in vivo is of high interest. The versatile coordination characteristics and mild labelling conditions of H₄noneunpaX, may be particularly well suited to applications in targeted alpha therapy (TAT) involving [²²⁵Ac]Ac³⁺ in combination with appropriate imaging radionuclides for dosimetry evaluations ([¹³⁵La]La³⁺, [¹⁵⁵Tb]Tb³⁺, [¹¹¹In]In³⁺, [⁴⁴Sc]Sc³⁺ etc).

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

### REFERENCES

The following references are of interest with respect to the subject matter described herein. Each of the following references is incorporated by reference in its entirety herein.
(1) Hancock, R. D.; Martell, A. E. Ligand Design for Selective Complexation of Metal Ions in Aqueous Solution. Chem. Rev. 1989, 89,1875-1914. https://doi.org/10.1021/cr00098a011.
(2) Hancock, R. D.; Wade, P. W.; Ngwenya, M. P.; de Sousa, A. S.; Damu, K. V. Ligand Design for Complexation in Aqueous Solution. 2. Chelate Ring Size as a Basis for Control of Size-Based Selectivity for Metal Ions. Inorg. Chem. 1990, 29, 1968-1974. https://doi.org/10.1021/ic00335a039.
(3) Price, E. W.; Orvig, C. Matching Chelators to Radiometals for Radiopharmaceuticals. Chem. Soc. Rev. 2014, 43, 260-290. https://doi.org/10.1039/c3cs60304k.
(4) Kostelnik, T. I.; Orvig, C. Radioactive Main Group and Rare Earth Metals for Imaging and Therapy. Chem. Rev. 2019, 119, 902-956. https://doi.org/10.1021/acs.chemrev. 8b00294.
(5) Price, E. W.; Cawthray, J. F.; Bailey, G. A.; Ferreira, C. L.; Boros, E.; Adam, M. J.; Orvig, C. H 4octapa: An Acyclic Chelator for 111 In Radiopharmaceuticals. J. Am. Chem. Soc. 2012, 134, 8670-8683. https://doi.org/10.1021/ja3024725.
(6) Spreckelmeyer, S.; Ramogida, C. F.; Rousseau, J.; Arane, K.; Bratanovic, I.; Colpo, N.; Jermilova, U.; Dias, G. M.; Dude, I.; Jaraquemada-Peláez, M. D. G.; Bénard, F.; Schaffer, P.; Orvig, C. P-NO2-Bn-H4neunpa and H4neunpa-Trastuzumab: Bifunctional Chelator for Radiometalpharmaceuticals and 111 In Immuno-Single Photon Emission Computed Tomography Imaging. Bioconjug. Chem. 2017, 28, 2145-2159. https://doi.org/10.1021/acs.bioconjchem.7b00311.
(7) Li, L.; Jaraquemada-Peláez, M. D. G.; Kuo, H. T.; Merkens, H.; Choudhary, N.; Gitschtaler, K.; Jermilova, U.; Colpo, N.; Uribe-Munoz, C.; Radchenko, V.; Schaffer, P.; Lin, K. S.; Bénard, F.; Orvig, C. Functionally Versatile and Highly Stable Chelator for 111 In and 177Lu: Proof-of-Principle Prostate-Specific Membrane Antigen Targeting. Bioconjug. Chem. 2019, 30, 1539-1553. https://doi.org/10.1021/acs.bioconjchem.9b00225.
(8) Wharton, L.; Kurakina, E.; Radchenko, V.; Schaffer, P.; Orvig, C. Chemical Promiscuity of Non-Macrocyclic Multidentate Chelating Ligands for Radiometal Ions: H4neunpa-NH2vs H4noneunpa. Inorg. Chem. 2021, 60, 4076-4092. https://doi.org/10.1021/acs.inorgchem.1c00152.
(9) Wang, X.; Jaraquemada-Peláez, M. D. G.; Rodríguez-Rodríguez, C.; Cao, Y.; Buchwalder, C.; Choudhary, N.; Jermilova, U.; Ramogida, C. F.; Saatchi, K.; Häfeli, U. O.; Patrick, B. O.; Orvig, C. H4octox: Versatile Bimodal Octadentate Acyclic Chelating Ligand for Medicinal Inorganic Chemistry. J. Am. Chem. Soc. 2018, 140 (45), 15487-15500. https://doi.org/10.1021/jacs.8b09964.
(10) Southcott, L.; Wang, X.; Wharton, L.; Yang, H.; Radchenko, V.; Kubeil, M.; Stephan, H.; de Guadalupe Jaraquemada-Peláez, M.; Orvig, C. High Denticity Oxinate-Linear-Backbone Chelating Ligand for Diagnostic Radiometal Ions [111 In]In3+ and [89Zr]Zr4+. Dalt. Trans. 2021, 50 (11), 3874-3886. https://doi.org/10.1039/d0dt04230g.
(11) Price, E. W.; Zeglis, B. M.; Cawthray, J. F.; Ramogida, C. F.; Ramos, N.; Lewis, J. S.; Adam, M. J.; Orvig, C. H4octapa-Trastuzumab: Versatile Acyclic Chelate System for 111In and 177Lu Imaging and Therapy. J. Am. Chem. Soc. 2013, 135 (34), 12707-12721. https://doi.org/10.1021/ja4049493.
(12) Jaraquemada-Peláez, M. D. G.; Wang, X.; Clough, T. J.; Cao, Y.; Choudhary, N.; Emler, K.; Patrick, B. O.; Orvig, C. H4octapa: Synthesis, Solution Equilibria and Complexes with Useful Radiopharmaceutical Metal Ions. Dalt. Trans. 2017, 42, 14647-14658. https://doi.org/10.1039/c7dt02343j.
(13) Hu, A.; Keresztes, I.; MacMillan, S. N.; Yang, Y.; Ding, E.; Zipfel, W. R.; Distasio, R. A.; Babich, J. W.; Wilson, J. J. Oxyaapa: A Picolinate-Based Ligand with Five Oxygen Donors That Strongly Chelates Lanthanides. Inorg. Chem. 2020, 59, 5116-5132. https://doi.org/10.1021/acs.inorgchem.0c00372.
(14) Kwekkeboom, D. J.; Kam, B. L.; Van Essen, M.; Teunissen, J. J. M.; Van Eijck, C. H. J.; Valkema, R.; De Jong, M.; De Herder, W. W.; Krenning, E. P. Somatostatin Receptor-Based Imaging and Therapy of Gastroenteropancreatic Neuroendocrine Tumors. Endocrine-Related Cancer. 2010. https://doi.org/10.1677/ERC-09-0078.
(15) Hennrich, U.; Kopka, K. Lutathera®: The First FDA- and EMA-Approved Radiopharmaceutical for Peptide Receptor Radionuclide Therapy. Pharmaceuticals. 2019. https://doi.org/10.3390/ph12030114.
(16) Müller, C.; Van Der Meulen, N. P.; Benešová, M.; Schibli, R. Therapeutic Radiometals beyond 177Lu and 90Y: Production and Application of Promising α-Particle, β-Particle, and Auger Electron Emitters. Journal of Nuclear Medicine. 2017, pp 91S-96S. https://doi.org/10.2967/jnumed.116.186825.
(17) Jain, A. K.; Raut, R.; Tuli, J. K. Nuclear Data Sheets for A=225. Nucl. Data Sheets 2009, 110 (6), 1409-1472. https://doi.org/10.1016/j.nds.2009.04.003.
(18) Shannon, R. D. Revised Effective Ionic Radii and Systematic Studies of Interatomic Distances in Halides and Chalcogenides. Acta Crystallogr. Sect. A 1976, A32, 751-767. https://doi.org/10.1107/S0567739476001551.
(19) Deblonde, G. J. P.; Zavarin, M.; Kersting, A. B. The Coordination Properties and Ionic Radius of Actinium: A 120-Year-Old Enigma. Coordination Chemistry Reviews. 2021, p 214130. https://doi.org/10.1016/j.ccr.2021.214130.
(20) Nica, N. Nuclear Data Sheets for A=155. Nucl. Data Sheets 2019, 160, 1-404. https://doi.org/10.1016/j.nds.2019.100523.
(21) Webster, B.; Ivanov, P.; Russell, B.; Collins, S.; Stora, T.; Ramos, J. P.; Köster, U.; Robinson, A. P.; Read, D. Chemical Purification of Terbium-155 from Pseudo-Isobaric Impurities in a Mass Separated Source Produced at CERN. Sci. Rep. 2019, 9 (1). https://doi.org/10.1038/s41598-019-47463-3.
(22) Müller, C.; Domnanich, K. A.; Umbricht, C. A.; Van Der Meulen, N. P. Scandium and Terbium Radionuclides for Radiotheranostics: Current State of Development towards Clinical Application. British Journal of Radiology. 2018. https://doi.org/10.1259/bjr.20180074.
(23) Naskar, N.; Lahiri, S. Theranostic Terbium Radioisotopes: Challenges in Production for Clinical Application. Frontiers in Medicine. 2021. https://doi.org/10.3389/fmed.2021.675014.
(24) Gans, P.; Sabatini, A.; Vacca, A. Determination of Equilibrium Constants from Spectrophometric Data Obtained from Solutions of Known PH: The Program PHab. Ann. Chim. 1999, 89 (1-2), 45-49.
(25) Gans, P.; Sabatini, A.; Vacca, A. Investigation of Equilibria in Solution. Determination of Equilibrium Constants with the HYPERQUAD Suite of Programs. Talanta 1996, 43 (10), 1739-1753. https://doi.org/10.1016/0039-9140(96)01958-3.
(26) Martell, A. E.; Smith, R. M. Critical Stability Constants; 1982. https://doi.org/10.1007/978-1-4615-6761-5.
(27) Blachot, J. Nuclear Data Sheets for A=111. Nucl. Data Sheets 2009, 110 (6), 1239-1407. https://doi.org/10.1016/j.nds.2009.04.002.
(28) Kurakina, E. S.; Wharton, L.; Hoehr, C.; Orvig, C.; Magomedbekov, E. P.; Filosofov, D.; Radchenko, V. Improved Separation Scheme for 44Sc Produced by Irradiation of NatCa Targets with 12.8 MeV Protons. Nucl. Med. Biol. 2022, 104-105, 22-27. https://doi.org/10.1016/j.nucmedbio.2021.11.002.
(29) Fiszbein, D. J.; Brown, V.; Thiele, N. A.; Woods, J. J.; Wharton, L.; Macmillan, S. N.; Radchenko, V.; Ramogida, C. F.; Wilson, J. J. Tuning the Kinetic Inertness of Bi3+Complexes: The Impact of Donor Atoms on Diaza-18-Crown-6 Ligands as Chelators for 213Bi Targeted Alpha Therapy. Inorg. Chem. 2021, 60 (12), 9199-9211. https://doi.org/10.1021/acs.inorgchem.1c01269.
(30) Pujales-Paradela, R.; Rodríguez-Rodríguez, A.; Gayoso-Padula, A.; Brandariz, I.; Valencia, L.; Esteban-Gómez, D.; Platas-Iglesias, C. On the Consequences of the Stereochemical Activity of the Bi(lii) 6s2 Lone Pair in Cyclen-Based Complexes. the [Bi(DO3A)] Case. Dalt. Trans. 2018, 47 (39), 13830-13842. https://doi.org/10.1039/c8dt02602e.
(31) Frisch, M. J.; Trucks, G. W.; Schlegel, H. B.; Scuseria, G. E.; Robb, M. A.; Cheeseman, J. R.; Scalmani, G.; Barone, V.; Petersson, G. A.; Nakatsuji, H.; Li, X.; Caricato, M.; Marenich, A. V.; Bloino, J.; Janesko, B. G.; Gomperts, R.; Mennucci, B.; Hratchian, H. P.; Ortiz, J. V.; Izmaylov, A. F.; Sonnenberg, J. L.; Williams-Young, D.; Ding, F.; Lipparini, F.; Egidi, F.; Goings, J.; Peng, B.; Petrone, A.; Henderson, T.; Ranasinghe, D.; Zakrzewski, V. G.; Gao, J.; Rega, N.; Zheng, G.; Liang, W.; Hada, M.; Ehara, M.; Toyota, K.; Fukuda, R.; Hasegawa, J.; Ishida, M.; Nakajima, T.; Honda, Y.; Kitao, O.; Nakai, H.; Vreven, T.; Throssell, K.; Montgomery, J. A., J.; Peralta, J. E.; Ogliaro, F.; Bearpark, M. J.; Heyd, J. J.; Brothers, E. N.; Kudin, K. N.; Staroverov, V. N.; Keith, T. A.; Kobayashi, R.; Normand, J.; Raghavachari, K.; Rendell, A. P.; Burant, J. C.; Iyengar, S. S.; Tomasi, J.; Cossi, M.; Millam, J. M.; Klene, M.; Adamo, C.; Cammi, R.; Ochterski, J. W.; Martin, R. L.; Morokuma, K.; Farkas, 0.; Foresman, J. B.; Fox, D. J. Gaussian 16, Rev. B.01. Gaussian, Inc., Wallingford, C T 2016.
(32) Tomasi, J.; Mennucci, B.; Cammi, R. Quantum Mechanical Continuum Solvation Models. Chem. Rev. 2005, 105, 2999-3094. https://doi.org/10.1021/cr9904009.
(33) Cao, X.; Dolg, M. Valence Basis Sets for Relativistic Energy-Consistent Small-Core Lanthanide Pseudopotentials. J. Chem. Phys. 2001, 115, 7348-7355. https://doi.org/10.1063/1.1406535.
(34) Cao, X.; Dolg, M. Segmented Contraction Scheme for Small-Core Lanthanide Pseudopotential Basis Sets. J. Mol. Struct. THEOCHEM2002, 581, 139-147. https://doi.org/10.1016/S0166-1280(01)00751-5.
(35) Stoll, H.; Metz, B.; Dolg, M. Relativistic Energy-Consistent Pseudopotentials-Recent Developments. J. Comput. Chem. 2002, 23 (8), 767-778. https://doi.org/10.1002/jcc.10037.
(36) Glendening, E. D.; Reed, A. E.; Carpenter, J. E.; Weinhold, F. NBO 3.0. QCPE Bull. 1990, 10.
(37) Chong, H. S.; Song, H. A.; Ma, X.; Milenic, D. E.; Brady, E. D.; Lim, S.; Lee, H.; Baidoo, K.; Cheng, D.; Brechbiel, M. W. Novel Bimodal Bifunctional Ligands for Radioimmunotherapy and Targeted MRI. Bioconjug. Chem. 2008, 19 (7), 1439-1447. https://doi.org/10.1021/bc800050x.
(38) Noor, A.; Van Zuylekom, J. K.; Rudd, S. E.; Roselt, P. D.; Haskali, M. B.; Yan, E.; Wheatcroft, M.; Hicks, R. J.; Cullinane, C.; Donnelly, P. S. Imaging Somatostatin Positive Tumors with Tyr3-Octreotate/Octreotide Conjugated to Desferrioxamine B Squaramide Radiolabeled with Either Zirconium-89 or Gallium-68. Bioconjug. Chem. 2021, 32(7), 1192-1203. https://doi.org/10.1021/acs.bioconjchem.1c00109.
(39) Borgna, F.; Barritt, P.; Grundler, P. V.; Talip, Z.; Cohrs, S.; Zeevaart, J. R.; Köster, U.; Schibli, R.; van der Meulen, N. P.; Müller, C. Simultaneous Visualization of 161Tb- and 177Lu-Labeled Somatostatin Analogues Using Dual-Isotope SPECT Imaging. Pharmaceutics 2021, 13 (4). https://doi.org/10.3390/pharmaceutics13040536.
(40) Rousseau, E.; Lau, J.; Zhang, Z.; Uribe, C. F.; Kuo, H. T.; Zhang, C.; Zeisler, J.; Colpo, N.; Lin, K. S.; Bénard, F. Effects of Adding an Albumin Binder Chain on [177Lu]Lu-DOTATATE. Nucl. Med. Biol. 2018, 66,10-17. https://doi.org/10.1016/j.nucmedbio.2018.08.001.
(41) Müller, C.; Fischer, E.; Behe, M.; Köster, U.; Dorrer, H.; Reber, J.; Haller, S.; Cohrs, S.; Blanc, A.; Grünberg, J.; Bunka, M.; Zhernosekov, K.; van der Meulen, N.; Johnston, K.; Türler, A.; Schibli, R. Future Prospects for SPECT Imaging Using the Radiolanthanide Terbium-155-Production and Preclinical Evaluation in Tumor-Bearing Mice. Nucl. Med. Biol. 2014, 41 (S). https://doi.org/10.1016/j.nucmedbio.2013.11.002.
(42) Roberts, M. S.; Magnusson, B. M.; Burczynski, F. J.; Weiss, M. Enterohepatic Circulation: Physiological, Pharmacokinetic and Clinical Implications. Clinical Pharmacokinetics. 2002, pp 751-790. https://doi.org/10.2165/00003088-200241100-00005.
(43) Minor, G.; Kapalka, J.; Fisher, C.; Paley, W.; Chen, K.; Kinakin, M.; Earle, I.; Moss, B.; Bricault, P.; Gottberg, A. Remote Handling Systems for the ISAC and ARIEL High-Power Fission and Spallation ISOL Target Facilities at TRIUMF. Nucl. Eng. Technol. 2020, 1-12. https://doi.org/10.1016/j.net.2020.09.024.
(44) Aluicio-Sarduy, E.; Hernandez, R.; Olson, A. P.; Barnhart, T. E.; Cai, W.; Ellison, P. A.; Engle, J. W. Production and in Vivo PET/CT Imaging of the Theranostic Pair 132/135La. Sci. Rep. 2019, 9 (1). https://doi.org/10.1038/s41598-019-47137-0.
(45) Ma, D.; McDevitt, M. R.; Finn, R. D.; Scheinberg, D. A. Breakthrough of 225Ac and Its Radionuclide Daughters from an 225Ac/213Bi Generator: Development of New Methods, Quantitative Characterization, and Implications for Clinical Use. Appl. Radiat. Isot. 2001, 55 (5), 667-678. https://doi.org/10.1016/S0969-8043(01)00062-8.
(46) Robertson, A. K. H.; McNeil, B. L.; Yang, H.; Gendron, D.; Perron, R.; Radchenko, V.; Zeisler, S.; Causey, P.; Schaffer, P. 232Th-Spallation-Produced 225Ac with Reduced 227Ac Content. Inorg. Chem. 2020, 59 (17), 12156-12165. https://doi.org/10.1021/acs.inorgchem.Oc01081.
(47) Loening, A. M.; Gambhir, S. S. AMIDE: A Free Software Tool for Multimodality Medical Image Analysis. Mol. Imaging 2003, 2 (3), 131-137. https://doi.org/10.1162/153535003322556877.
(48) Gran, G. Determination of the Equivalence Point in Potentiometric Titrations. Part II. Analyst 1952, 77 (920), 661-670. https://doi.org/10.1039/AN9527700661.
(49) Huang, P. W. Understanding the Stability Trend Along Light Lanthanide Complexes with an Ehtylenediamine-Type Ligand: A Quantum Chemical Study. ChemistrySelect 2019, 4, 12368-12374. https://doi.org/10.1002/slct.201902887.
(50) Kovács, A.; Varga, Z. Metal-Ligand Interactions in Complexes of Cyclen-Based Ligands with Bi and Ac. Struct. Chem. 2021, 32 (5), 1719-1731. https://doi.org/10.1007/s11224-021-01816-9.
(51) Hanwell, M. D.; Curtis, D. E.; Lonie, D. C.; Vandermeerschd, T.; Zurek, E.; Hutchison, G. R. Avogadro: An Advanced Semantic Chemical Editor, Visualization, and Analysis Platform. J. Cheminform. 2012, 4 (8). https://doi.org/10.1186/1758-2946-4-17.
(52) Lu, T.; Chen, F. Multiwfn: A Multifunctional Wavefunction Analyzer. J. Comput. Chem. 2012, 33 (5), 580-592. https://doi.org/10.1002/jcc.22885.
(53) Branderhorst, W.; Vastenhouw, B.; Beekman, F. J. Pixel-Based Subsets for Rapid Multi-Pinhole SPECT Reconstruction. Phys. Med. Biol. 2010, 55(7), 2023-2034. https://doi.org/10.1088/0031-9155/55/7/015.
(54) Foster, Henry L.; Small, J. David.; Fox, J. G. The Mouse in Biomedical Research; Academic Press: New York, 1983.
(55) Gran, G. Determination of the Equivalence Point in Potentiometric Titrations. Part II. Analyst 1952, 77 (920), 661-670. https://doi.org/10.1039/AN9527700661.
(56) Alderighi, L.; Gans, P.; Ienco, A.; Peters, D.; Sabatini, A.; Vacca, A. Hyperquad Simulation and Speciation (HySS): A Utility Program for the Investigation of Equilibria Involving Soluble and Partially Soluble Species. Coord. Chem. Rev. 1999, 184 (1), 311-318. https://doi.org/10.1016/S0010-8545(98)00260-4.
(57) Harris, W. R.; Carrano, C. J.; Raymond, K. N. Spectrophotometric Determination of the Proton-Dependent Stability Constant of Ferric Enterobactin. J. Am. Chem. Soc. 1979, 101 (8), 2213-2214. https://doi.org/10.1021/ja00502a053.
(58) Milenic, D. E.; Roselli, M., Mirzadeh, S.; Pippin, C. G.; Gansow, O. A.; Colcher, D; Brechbiel, M. W; Schlom, J. In Vivo Evaluation of Bismuth-Labeled Monoclonal Antibody Comparing DTPA-Derived Bifunctional Chelates. Cancer Biother. & Radiopharm. 2001, 16(2), 133-146. http://doi.org/10.1089/108497801300189227

1. A chelator having the structure (I), wherein each R₁ is independently OH, NH or SH, and X is O, S, or NR₃, wherein R₃ is H or CH₂C(═O)R₁:

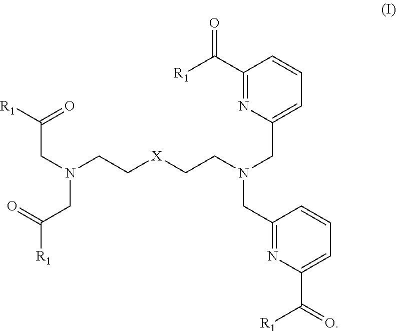

2. A chelator as defined in claim 1 having the following structure:

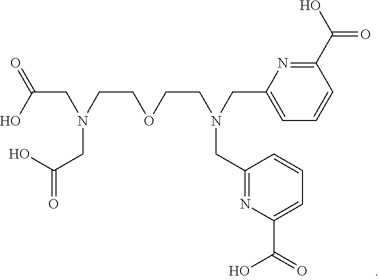 [SMILES: O=C(O)CN(CCOCCN(Cc1cccc(C(=O)O)n1)Cc1cccc(C(=O)O)n1)CC(=O)O]

3. A chelator having the structure (III) wherein each R₁ is independently OH, NH or SH or a functional group; each R₂ is independently H or a functional group; each R₄ is independently H or a functional group, or both R₄ together form a cyclohexyl moiety; each R₅ is independently H or a functional group, or both R₅ together form a cyclohexyl moiety; and X is O, S, or NR₃, wherein one or more of the R₁, R₂, R₄ or R₅ groups is a functional group that allows coupling of the chelator to a biological targeting moiety, and wherein R₃ is H or CH₂C(═O)R₁:

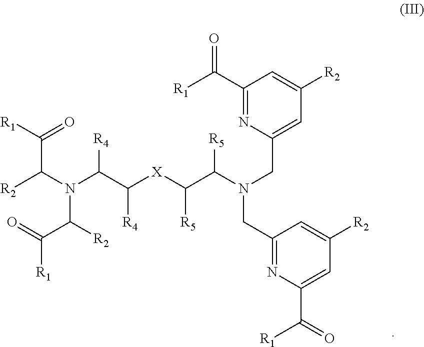

4. A chelator as defined in claim 3 having the structure (II) wherein each R₁ is independently OH, NH or SH or a functional group, each R₂ is independently H or a functional group, and X is O, S, or NR₃, wherein one or more of the R₁ or R₂ groups is a functional group that allows coupling of the chelator to a biological targeting moiety, and wherein R₃ is H or CH₂C(═O)R₁:

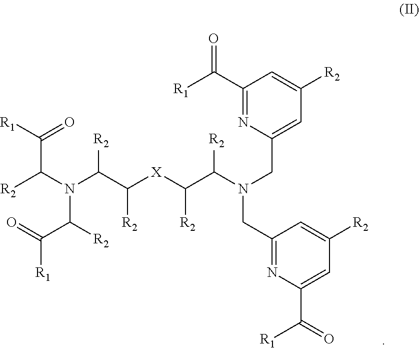

5. A chelator as defined in claim 3, wherein the functional group is a carboxyl, an ester, an amide, an imide, a thioamide, a thioester, or a guanidinium group.

6. A chelate comprising a radiometal and a chelator as defined in claim 1.

7. An in vivo targeting construct comprising a biological targeting moiety and a chelator as defined in claim 3, wherein one R₁ group, one R₂, one R₄ group or one R₅ group is coupled to the biological targeting moiety, the in vivo targeting construct optionally further comprising a linker interposing the chelator and the biological targeting moiety, wherein the linker comprises a C₁-C₁₀ hydrocarbon linker that is optionally substituted with one or more heteroatoms or has one or more substituents, an aromatic linker, a cationic linker, an anionic linker, an amino acid linker having between one and ten amino acids, a cyclized amino acid linker, a PEG linker, a cyclized ring linker, an aromatic linker, or a click chemistry linker.

8. (canceled)

9. An in vivo targeting construct as defined in claim 7, further comprising a radiometal chelated by the chelator.

10. A chelate or an in vivo targeting construct as defined in claim 9, wherein the radiometal comprises ²²⁵Ac, ²²⁷Th, ²²⁶Th, ²¹¹At, ⁴⁴Sc, ⁹⁰Y, ⁸⁹Zr, ¹⁷⁷Lu, ¹¹¹In, ^{86/89/90}Y, ²¹¹At, ²¹¹Fr, ^{212/213}Bi, ¹⁵³Sm, ^{161/166}Ho, ^{165/166}Dy, ^{161/155}Tb, ¹⁴⁰La, ^{142/143/145}Pr, ¹⁵⁹Gd, ^{169/175}Yb, ^{167/170}Tm, ¹⁶⁹Er, ¹⁴⁹Pm, ¹⁵⁰Eu, ⁶⁸Ga, ¹³⁷Cs, or ¹⁴¹Ce; wherein the radiometal optionally comprises ²²⁷Th, ²²⁵Ac, ¹⁵⁵Tb, ¹⁷⁷Lu, ¹¹¹In, ¹³²La, ²³⁵La, ⁹⁰Y, ⁶⁸Ga, ⁴⁴Sc, ²⁰³Pb, or ²¹²Pb.

11. (canceled)

12. A chelate or an in vivo targeting construct as defined in claim 9, wherein the radiometal comprises ²²⁵Ac, ¹⁵⁵Tb, ¹⁷⁷Lu, ¹¹¹In, ¹³²La, ²³⁵La, or ⁴⁴Sc, optionally wherein the radiometal comprises ²²⁵Ac.

13. (canceled)

14. An in vivo targeting construct as defined in claim 7, wherein the targeting moiety comprises a hapten, an antigen, an aptamer, an affibody, an enzyme, a protein, a peptide, an antibody, an antigen-binding fragment of an antibody, a peptidomimetic, a receptor ligand, a steroid, a hormone, a growth factor, a cytokine, a molecule that recognizes cell surface receptors, a lipid, a lipophilic group, a carbohydrate; wherein the antigen-binding fragment of an antibody optionally comprises an Fab fragment, an F(ab′)₂ fragment, a Fv fragment, a scFv fragment, a minibody, or a diabody.

15. (canceled)

16. An in vivo radioisotope targeting construct as defined in claim 7, wherein the biological targeting moiety comprises A33 antibody, dihydrotestosterone (DHT), HuMab-5B1, girentuximab, AMG211 bispecific T-cell engager, IAB22M2C minibody, rituximab, obinutuzumab, U36 antibody, plerixafor, pentixafor, NFB, ipilimumab, erlotinib, PD153035, afatinib, cetuximab, panitumumab, ABY-025 affibody, HER2-nanobody, trastuzumab, pertuzumab, GSK2849330, lumretuzumab, 4FMFES, FAPI-04, FAPI-21, FAPI-46, galactose, CB-TE2A-AR06 peptide (with H₄noneunpaX substituted for DOTA), BAY 864367 peptide (with H₄noneunpaX-bound ligand label instead of 18F labeling), RM2 peptide (with H₄noneunpaX substituted for DOTA), SB3 peptide (with H₄noneunpaX substituted for DOTA), RM26 peptide, BBN-RGD peptide, Aca-BBN peptide, NeoBOMB1 peptide (with H₄noneunpaX substituted for DOTA), exendin-4 peptide, glucose, codrituzumab, EF5, MISO, AZA, HX4, ASTM, LLP2A, peptidomimetic, galacto-RGD peptide, FPP(RGD)2 peptide, RGD-K5 peptide, fluciclatide, alfatide-1, alfatide-II, PRGD2 peptide, αvβ6-BP peptide, CycMSHhex targeting peptides, MMOT0530A antibody, SP peptide, neurotensin, PARPi, a PSMA peptidomimetic, DCFPyL, DCFBC, HuJ591 antibody, durvalumab, nivolumab, pembrolizumab, BMS-986192 adnectin, atezolizumab, MSTP2109A antibody, TATE peptide (octreotate), TOC peptide, NOC peptide, JR11, thymidine, fresolimumab, or bevacizumab; or wherein the biological target targeted by the in vivo targeting construct comprises: a tumor associated antigen, A33 transmembrane qlycoprotein, androgen receptor (AR), CA19.9, carbonic anhydrase 9 (CA-IX), carcinoembryonic antigen, CD8, CD20, CD44v6, C—X—C chemokine receptor type 4 (CXCR4), cytotoxic T-lymphocyte-associated protein 4 (CTLA-4), epidermal growth factor receptor (EGFR), epidermal growth factor receptor 2 (ERBB2), epidermal growth factor receptor 3 (ERBB3), estrogen receptor (ER), fibroblast activation protein a, gastrin-releasing peptide receptor (GRPR), glucagon-like peptide 1 receptor (GLP-1R), glypican 3, integrin α4β1, integrin αvβ3, integrin αvβ6, melanocortin-1 receptor (MC1R), mesothelin, neurokinin1 receptor (NK1R), neurotensin 1 receptor (NTS1R), poly(ADP-ribose) polymerase 1 (PARP1), prostate-specific membrane antigen (PSMA), programmed cell death protein (PD-1), programmed death-ligand 1 (PD-L1), six-transmembrane epithelial antigen of prostate-1 (STEAPI), somatostatin receptor 2 (SSTR2), thymidine kinase, transforming growth factor-beta (TGF-β), or vascular endothelial growth factor receptor (VEGFR).

17. (canceled)

18. An in vivo targeting construct as defined in claim 7, wherein the biological targeting moiety comprises octreotate (TATE).

19. (canceled)

20. A method of delivering a radioisotope to a selected location within the body of a mammalian subject, the method comprising:
administering an in vivo targeting construct as defined in claim 7 bearing the radioisotope to the mammalian subject, optionally wherein the mammalian subject is a human.

21. A method as defined in claim 20, further comprising allowing the targeting moiety of the in vivo targeting construct to enhance the accumulation of the radioisotope at the selected location within the body relative to other locations in the body to selectively deliver radiation to the selected location.

22. A method as defined in claim 20, further comprising a step of forming a chelate comprising the radioisotope and the in vivo targeting construct prior to the administering step, wherein the step of forming the chelate comprises combining the in vivo targeting construct with the radioisotope at a temperature of between about 10° C. and about 65° C. for an incubation period.

23. A method as defined in claim 22, wherein;
the temperature is between about 15° C. and about 25° C. during the incubation period;
the incubation period is between about 5 minutes and about 30 minutes;
the combining step is carried out at a pH in the range of about 5.0 to about 7.4; and/or
the combining step is carried out in aqueous solution that is substantially free of alcohol.

24. (canceled)

25. (canceled)

26. (canceled)

27. A method as defined in claim 20, further comprising carrying out an imaging procedure to evaluate the localization of the in vivo targeting construct within the body, wherein the imaging procedure optionally comprises positron emission tomography (PET) imaging or single-photon emission computerized tomography (SPECT) imaging.

28. A method as defined in claim 20, wherein the in vivo targeting construct is used to cause cell death at the selected location within the body by exposing the cells to radiation from the radioisotope, optionally wherein the cells are cancer cells.

29. (canceled)

30. A method as defined in claim 28, wherein the radiation comprises alpha radiation or beta radiation.

31. (canceled)
